(19) United States
(12) Patent Application Publication
(10) Pub. No.: US 2022/0280530 A1
(43) Pub. Date: Sep. 8, 2022

(54) PREVENTION OR TREATMENT OF DISEASES AND DISORDERS ASSOCIATED WITH TISSUE DAMAGE

(71) Applicant: Xiaoxiang Li, Hefei (CN)

(72) Inventor: Xiaoxiang Li, Hefei (CN)

(73) Assignee: Anhui Newstar Pharmaceutical Development Co., Ltd., Hefei (CN)

(21) Appl. No.: 17/632,500

(22) PCT Filed: Jul. 31, 2020
(86) PCT No.: PCT/IB2020/057296

§ 371 Date: Feb. 2, 2022

Related U.S. Application Data
(60) Provisional application No. 62/884,241, filed on Aug. 8, 2019.

(51) Int. Cl. A61K 31/5513; A61K 31/551; A61K 31/5517; A61P 9/10

(52) U.S. Cl. CPC A61K 31/5513; A61K 31/551; A61K 31/5517; A61P 9/10

(57) ABSTRACT

The present invention provides a method of preventing or treating diseases and disorders associated with tissue damages using an MST1/2 protein kinase inhibitor. The MST1/2 protein kinase inhibitor may be administered in its prodrug or salt forms. A bioavailability enhancing agent or an absorption enhancing agent may be used in conjunction with the MST1/2 protein kinase inhibitor.

### TECHNICAL FIELD

The present invention relates to the field of prevention or treatment of tissue damages, in particular, it relates to using inhibitors of MST1/2 protein kinases for prevention or treatment of diseases and disorders associated with tissue damages, for promotion of tissue or organ regeneration and repair, for prevention or treatment of diseases caused by inflammation, and for prevention or treatment of neurological disorder related diseases and ischemic diseases.

### BACKGROUND TECHNOLOGY

How organ size is controlled in multicellular organisms is a fundamental question in biology. It has been proposed that the mammalian target of rapamycin (mTOR) pathway and the Hippo-YAP pathway control organ size by affecting cell size and cell number, respectively (Lee et al, Annu Rev Pharmacol Toxicol (2007) 47:443-467; Zhao et al, Genes Dev (2010) 24:862-874). The Hippo-YAP pathway was initially defined by genetic studies in Drosophila, in which mosaic mutation of the Hippo-YAP pathway genes resulted in tissue overgrowth (Pan, Genes Dev (2007) 21:886-897).

Function of the Hippo-YAP pathway in organ size regulation is conserved in mammals as demonstrated by studies using genetically modified mouse models (Camargo et al, Curr Biol (2007) 17, 2054-2060; Dong et al, Cell (2007) 130:1120-1133; Heallen et al, Science (2011) 332:458-461; Lee et al, Proc Natl Acad Sci USA (2010) 107:8248-8253; Lu et al, Proc Natl Acad Sci USA (2010) 107:1437-1442; Song et al, Proc Natl Acad Sci USA (2010) 107:1431-1436).

Core components of the mammalian Hippo-YAP pathway include a kinase cascade of mammalian sterile 20-like kinase-1/2 (MST1/2) and LATS1/2. MST1/2, in complex with its regulatory protein Salvador (SAV1), phosphorylates and activates LATS1/2 kinases, which also form a complex with its regulatory protein MOB1 (Zhao et al, Genes Dev (2010) 24:862-874). SAV1 forms complexes with MST1/2, whereas MOB kinase activator 1A (MOB1A) and MOB1B interact with LATS1/2. When the Hippo pathway is activated, MST1/2 activate LATS1/2 and MOB1A/1B by phosphorylation. Subsequently, LATS1/2 directly phosphorylate YAP and TAZ. Phosphorylation inhibits YAP and TAZ activities by activating a phosphodegron that is targeted by β-transducin repeat-containing protein (β-TrCP), leading to the degradation of YAP and TAZ proteins. As a result, YAP and TAZ accumulate in the nucleus and promote gene expression when the Hippo pathway is not active (FIG. 1).

MST1/2 are a ubiquitously expressed serine/threonine kinase, which belongs to a mammalian sterile 20 (STE 20)—like kinase family consisting of PAKI, MST1, MST2, KHS, GCK, SOK1, NIK, HPK1 AND SPS1. Increasing lines of evidence suggests that MST1/2 and other STE20-like family kinases play an important role in mediating apoptosis. MST1/2 are activated by some pro-apoptotic stimuli in fibroblastic and lymphocytic cell lines. MST1/2 are cleaved by caspases and this cleavage increases kinase activities of MST1/2, which in turn activates caspase 3, thereby constituting a powerful amplification loop of apoptotic response (Cinar et al., EMBO J. (2007) 26:4523-4534; Song and Lee, Cell Signal. (2008) 20:892-906).

Conditional knockout of MST1/2 protein kinases can promote liver regeneration (Zhou et al. Cancer Cell (2009) 16:425-438), and immunosuppression (Mou et al. J. Exp. Med. (2012) 209:741-759). Reducing MST1/2 protein level or enzymatic activity helps reducing neuronal cell death, and thus be useful for prevention and treatment of neurological disorders or neurodegenerative diseases, including Alzheimer's disease, multiple sclerosis, Parkinson's disease, stroke, etc. (Lehtinen et al. Cell (2006) 125:987-1001); and oxidizing stress-related myocardial ischemia and peripheral ischemia (US 2008/0242608).

Thus, the present invention provides a method for preventing or treating tissue damage related diseases and disorder using small molecule inhibitors of MST1/2 protein kinases.

### SUMMARY OF INVENTION

The invention provides a method of preventing or treating a disease or disorder associated with tissue damage using a MST1/2 protein kinase inhibitor, said method comprising a step of administering the MST1/2 protein kinase inhibitor to a subject having the disease or disorder, wherein the MST1/2 protein kinase inhibitor is administered at a dose of from about 0.1 mg/kg to about 100 mg/kg based on the bodyweight of the subject and at a frequency of once in a period of from 6 hours to 20 days, and the MST1/2 protein kinase inhibitor has a formula:

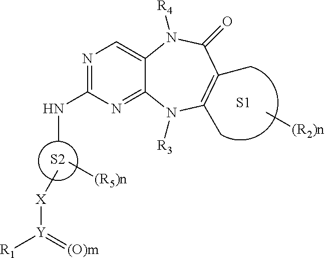

or a stereoisomer of the above compounds, a prodrug thereof, a pharmaceutically acceptable salt thereof, or a pharmaceutically acceptable solvate thereof. The definition of the substituents and symbols are described in detail below.

In the previous embodiment, the disease or disorder associated with tissue damage may be a trauma to brain, a trauma to spinal cord, a trauma to peripheral nerves, a trauma to retinal or a trauma to heart. In one embodiment, the trauma to brain may be ischemic stroke, blunt trauma, or subarachnoid hemorrhage. In another embodiment, the trauma to spinal cord may be spinal cord ischemia or spinal cord blunt force trauma. In yet another embodiment, the trauma to peripheral nerves may be sciatic nerve injury, diabetic neuropathy, or carpal tunnel syndrome. In yet another embodiment, the trauma to retinal may be macular edema, diabetic retinopathy, or glaucoma. In yet another embodiment, the trauma to heart may be myocardial infarct, or chronic heart failure.

In any one of the previous embodiments, the disease or disorder associated with tissue damage may be an organ failure. In one embodiment, the organ failure may be selected from diabetes mellitus type I or II, nephrosis, fatty liver diseases, failure of gonads, failure of pancreas, failure of kidney, failure of heart, failure of lung, failure of liver, and failure of bowel.

In any one of the previous embodiments, the disease or disorder associated with tissue damage may be a disease or disorder caused by exposure to a toxic agent. In one embodiment, the toxic agent may be selected from chemotherapeutic agents, chemical agents and radiation agents.

In any one of the previous embodiments, the disease or disorder associated with tissue damage may be an inflammatory disease. In one embodiment, the inflammatory disease may be selected from sepsis, inflammatory bowel diseases, Crohn's disease, ulcerative colitis, ileitis, enteritis, and acute nephritis.

In any one of the previous embodiments, the disease or disorder associated with tissue damage may be a degenerative disease. In one embodiment, the degenerative disease may be selected from muscular dystrophies, myotonic dystrophy, and neurodegenerative diseases.

In any one of the previous embodiments, the MST1/2 protein kinase inhibitor may be administered prior to onset of the disease or disorder associated with tissue damage, during development of the disease or disorder associated with tissue damage, and/or after the disease or disorder associated with tissue damage has developed.

In any one of the previous embodiments, the MST1/2 protein kinase inhibitor may be administered from one minute to about 24 hours, or from about 5 minutes to about 10 hours, or from about 5 minutes to about 5 hours prior to onset of the disease or disorder associated with tissue damage.

In any one of the previous embodiments, the MST1/2 protein kinase inhibitor may be administered at a dose of from about 1 to about 10 mg/kg bodyweight.

In any one of the previous embodiments, the MST1/2 protein kinase inhibitor may be administered at a dose of from about 0.1 to about 10 mg/kg bodyweight.

In any one of the previous embodiments, the MST1/2 protein kinase inhibitor may be administered at a dose of from about 10 to about 100 mg/kg bodyweight.

In any one of the previous embodiments, the MST1/2 protein kinase inhibitor may be administered at a frequency of once in a period of from 8 hours to 10 days, from 12 hours to 7 days, or from 12 hours to 3 days, or from 12 hours to 24 hours.

In any one of the previous embodiments, the MST1/2 protein kinase inhibitor may be administered through intravenous injection, intravenous infusion, intravenous drip, subcutaneous injection, sublingual administration, or oral administration to the subject. In one embodiment, the the MST1/2 protein kinase inhibitor may be administered by intravenous injection or intravenous infusion.

In any one of the previous embodiments, the method may further comprise a step of administering an additional active ingredient that is effective in treating a degenerative disease or tissue injury. In one embodiment, the additional active ingredient and the MST1/2 protein kinase inhibitor may be administered simultaneously or in separate sequential administrations. In another embodiment, the additional active ingredient and the MST1/2 protein kinase inhibitor may be administered in separate sequential administrations that are less than half an hour, 1 hour, 2 hours, 4 hours, 6 hours, 8 hours, or 10 hours apart. In yet another embodiment, the additional active ingredient may be selected from chemoprotective agents, myeloprotective agents, anti-apoptotic agents, and pro-proliferative agents.

In any one of the previous embodiments, the MST1/2 protein kinase inhibitor may have one of formulas I, II, III, IV:

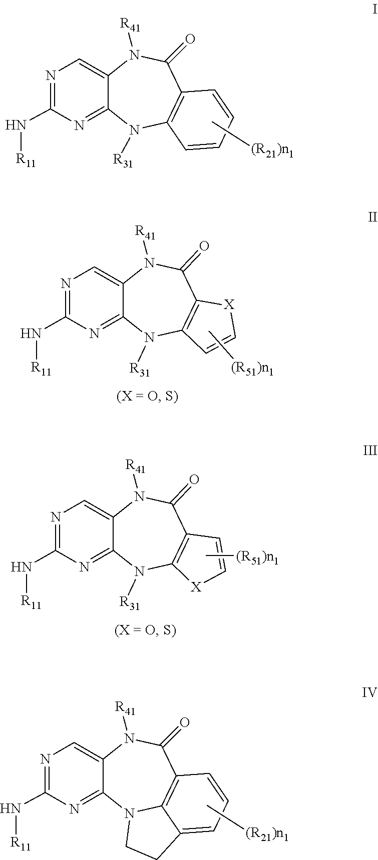

wherein definition of the substituents and symbols are described in detail below.

In any one of the previous embodiments, the MST1/2 protein kinase inhibitor may be selected from the compounds in Tables 1-4.

In any one of the previous embodiments, the MST1/2 protein kinase inhibitor may be selected from:

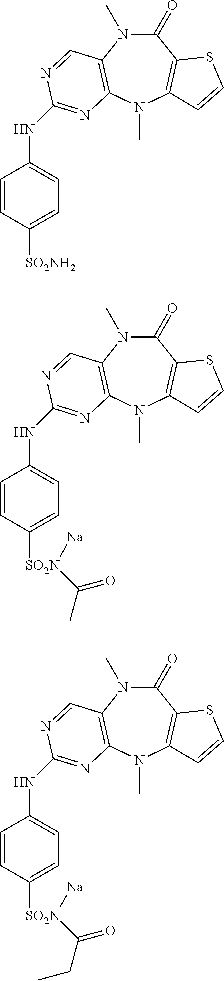

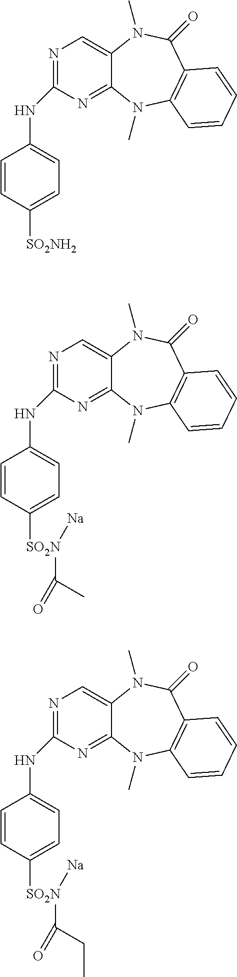

In any one of the previous embodiments, the MST1/2 protein kinase inhibitor may be administered in its prodrug form or its salt form.

In any one of the previous embodiments, the MST1/2 protein kinase inhibitor may be administered in a pharmaceutical composition comprising a pharmaceutical excipient selected from carriers, diluents, fillers, buffers, bulking agents, stabilizers, and solubilizers.

In any one of the previous embodiments, the MST1/2 protein kinase inhibitor may be administered in a pharmaceutical composition comprising a solubilizing agent, an emulsifier, or a surfactant.

In any one of the previous embodiments, the MST1/2 protein kinase inhibitor may be administered in a pharmaceutical composition comprising a bioavailability enhancing agent or an absorption enhancing agent.

### BRIEF DESCRIPTION OF DRAWINGS

FIG. 1 is a schematic presentation of the Hippo pathway where MST1/2 protein kinases play a pivotal role.

FIG. 2 shows inhibition of Mob1 phosphorylation by MST1/2 protein kinase inhibitors at a dose dependent manner.

FIG. 3 shows the Mob1 phosphorylation inhibition curve by an MST1/2 protein kinase inhibitor.

FIGS. 4A-4D show reduction of H₂O₂ induced neonatal rat cardiomyocytes (NRCM) death by the MST1/2 protein kinase inhibitors.

FIGS. 5A-5D show reduction of isoproterenol (ISO) induced cardiomyocytes death by the MST1/2 protein kinase inhibitors.

FIGS. 6A-6D show reduction of doxorubicin (DOX) induced cardiomyocytes death by the MST1/2 protein kinase inhibitors.

FIG. 7A shows inhibition of apoptosis in cardiomyocytes induced by H₂O₂.

FIG. 7B shows inhibition of apoptosis in cardiomyocytes induced by ISO.

FIG. 7C shows inhibition of apoptosis in cardiomyocytes induced by DOX.

FIG. 8 shows a study design for inhibition of heart atrophy by the MST1/2 protein kinase inhibitors in mice.

FIG. 9 shows percentages of survivals over the study period after treatment by Dox, optionally and additionally with the MST1/2 protein kinase inhibitors.

FIG. 10 shows ratio of heart weight over bodyweight of the mice after treatment by Dox, optionally and additionally with the MST1/2 protein kinase inhibitors.

FIG. 11 shows heart ejection fraction (EF %) of the mice after treatment by Dox, optionally and additionally with the MST1/2 protein kinase inhibitors.

FIG. 12 shows the rate of incorporation of EdU into cardiomyocytes of mice after treatment with the MST1/2 protein kinase inhibitors.

FIGS. 13 and 14A show inhibition of infarct size in ischemic stroke mice after treatment with the MST1/2 protein kinase inhibitors.

FIG. 14B shows protection of neurological functions in ischemic stroke mice after treatment with the MST1/2 protein kinase inhibitors.

FIGS. 15A-15B show inhibition of VCAM-1 expression in human umbilical vein endothelial cells (HUVEC) as induced by TNFα using the MST1/2 protein kinase inhibitor X1.

FIGS. 16A-16B show inhibition of VCAM-1 expression in HUVEC as induced by TNFα using the MST1/2 protein kinase inhibitor Y1.

FIGS. 17A-17D show inhibition of expression of inflammatory markers in HUVEC induced by TNFα using the MST1/2 protein kinase inhibitor X1.

FIGS. 18A-18D show inhibition of expression of inflammatory markers in HUVEC induced by TNFα using the MST1/2 protein kinase inhibitor Y1.

FIGS. 19A-19B show inhibition of cell adhesion induced by TNFα using the MST1/2 protein kinase inhibitor X1.

FIGS. 20A-20B show inhibition of cell adhesion induced by TNFα using the MST1/2 protein kinase inhibitor YL.

FIGS. 21A-21D show inhibition of expression of inflammatory markers induced by lipopolysaccharides (LPS) using the MST1/2 protein kinase inhibitor X1.

### DEFINITIONS

In order to facilitate understanding of the examples provided herein, certain frequently occurring terms are defined herein.

In connection with a measured quantity, the term “about” as used herein refers to the normal variation in that measured quantity that would be expected by a skilled person making the measurement and exercising a level of care commensurate with the objective of the measurement and the precision of the measuring equipment used. Unless otherwise indicated, “about” refers to a variation of +/−10%, +/−5%, or +/−2% of the value provided.

The term “administering” or “administration” as used herein refers to local and systemic administration of an inhibitor of MST1/2 protein kinase, e.g., including enteral, parenteral, pulmonary, and topical/transdermal administration. Routes of administration for an inhibitor of MST1/2 as described herein include, e.g., oral (per os (P.O.)) administration, nasal or inhalation administration, administration as a suppository, topical contact, transdermal delivery (e.g., via a transdermal patch), intrathecal (IT) administration, intravenous (“i.v.”) administration, intraperitoneal (“i.p.”) administration, intramuscular (“im”) administration, intratumoral administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The term “cancer” as used herein refers to any abnormal growth exhibiting malignant properties: the ability (1) to grow and divide without respect to normal limits, (2) to invade and destroy adjacent tissues, and (3) in some instances, spread to other locations in the body. Cancer includes cancers or neoplastic disorders of the central nervous system, peripheral nervous system, gastrointestinal/digestive system, genitourinary system, gynecological, head and neck, hematological/blood, musculoskeletal/soft tissue, respiratory, and breast. Further examples of cancers or neoplastic disorders include, but are not limited to, those of the brain (astrocytoma, gliobastoma, glioma), spinal cord, pituitary gland, breast (Infiltrating cancers, Pre-invasive cancers, inflammatory cancers, Paget's Disease, Metastatic and Recurrent Breast Cancer), blood (Hodgkin's Disease, Leukemia, Multiple Myeloma, Lymphoma), Lymph node cancer, Lung (Adenocarcinoma, Oat Cell lung cancer, Non-small Cell lung cancer, Small Cell lung cancer, Squamous Cell lung cancer, Mesothelioma), skin (melanoma, basal cell skin cancer, squamous cell skin cancer, Kapsosis Sarcoma), Bone Cancer (Ewings Sarcoma, Osteosarcoma, Chondrosarcoma), head and neck (laryngeal, pharyngeal (nasal cavity & sinus cavity), and esophageal cancers), oral (jaw, salivary gland, throat, thyroid, tongue, and tonsil cancers), eye, gynecological (Cervical, Endometrial, Fallopian, Ovarian, Uterine, Vaginal, and Vulvar), genitourinary (bladder, kidney, penile, prostate, testicular, and urinary cancers), adrenal (cortical adenoma, cortical carcinoma, pheochromocytoma) and gastrointestinal (appendix, bile duct (extrahepatic bile duct) colon, gallbladder, gastric, intestinal, colon, liver, pancreatic, rectal, and stomach cancers) as well as those listed below: (Fishman et al., 1985, Medicine, 2d Ed., J. B. Lippincott Co., Philadelphia): Leukemia: acute leukemia, acute lymphocytic leukemia, acute myelocytic leukemia, myeloblastic, promyelocytic, myelomonocytic, monocytic erythroleukemia, chronic leukemia, chronic myelocytic (granulocytic) leukemia, chronic lymphocytic leukemia, Polycythemia vera, Gastric carcinoma; Lymphoma (malignant and non-malignant): Hodgkin's disease, non-Hodgkin's disease, Multiple myeloma, Waldenstrom's macroglobulinemia, Heavy chain disease; Solid tumors sarcomas and carcinomas: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, oral squamous cell carcinoma, hepatocellular carcinoma, basal cell carcinoma, adenocarcinoma, sweat gland carcinoma, sebaceous gland carcinoma, papillary carcinoma; papillary adenocarcinomas: cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, cervix adenocarcinoma, uterine cancer, testicular tumor, lung carcinoma, small cell lung carcinoma, non-small cell lung adenocarcinoma, bladder carcinoma, epithelial carcinoma, glioma, malignant glioma, glioblastoma, multiforme astrocytic gliomas, medulloblastoma, craniopharyngioma, ependymoma, pinealoma, hemangioblastoma, acoustic neuroma, oligodendroglioma, meningioma, melanoma, neuroblastoma, or retinoblastoma.

The term “chemical-induced toxicity” as used herein refers to injury induced by a chemical agent to a cell or tissue arising from exposure to the chemical agent. Phenotypically, chemical-induced injury includes one or more of the following: structural chemical injury to a cell or tissue, inflammation, fibroproliferative tissue effects, adverse tissue remodeling, (e.g., increased neutrophil infiltration), relative to that seen in a cell or tissue not exposed to a chemical.

The term “chemotherapeutic agent” as used herein refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMF®); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN®) combined with 5-FU and leucovorin.

The term “individual” or “subject” or “patient” as used herein refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human, which is commonly termed “patients”.

The term “inflammatory diseases” as used herein refers to various diseases having an inflammatory component. Non-limiting examples include, but are not limited to, appendicitis, blepharitis, bronchitis, bursitis, cervicitis, cholangitis, cholecystitis, chorioamnionitis, conjunctivitis, cystitis, dacryoadenitis, dermatitis, endocarditis, endometritis, epicondylitis, epididymitis, fibrositis, gastritis, gingivitis, glossitis, hidradenitis suppurativa, iritis, laryngitis, mastitis, myocarditis, myositis, nephritis, omphalitis, oophoritis, orchitis, osteitis, otitis, parotitis, pericarditis, peritonitis, pharyngitis, pleuritis, phlebitis, pneumonitis (pneumonia), prostatitis, pyelonephritis, rhinitis, salpingitis, sinusitis, stomatitis, synovitis, tonsillitis, uveitis, urethritis, vaginitis, vulvitis, asthma, systemic lupus erythematosus, myasthenia gravis, tendonitis, angiitis, chronic bronchitis, pancreatitis, osteomyelitis, arthritis (rheumatoid and psoriatic), glumeronephritis, optic neuritis, temporal arteritis, encephalitis, meningitis, traverse myelitis, dermatomyositis, polymyositis, necrotizing fasciitis, hepatitis, necrotizing entercolitis, pelvic inflammatory disease, inflammatory bowel disease (ulcerative colitis, Crohn's disease, ileitis, and enteritis), proctitis, vasculitis, vascular stenosis, restenosis, hypotension, Type-1 diabetes, Kawasaki disease, Decum's disease, chronic obstructive pulmonary disease, psoriasis, artherosclerosis, scleroderma, Sjogren's syndrome, mixed connective tissue disease, rosacea, gastric ulcers, duodenal ulcers, Alzheimer's disease, adult onset Still's disease, acute retinal pigment epitheliitis, Tietze's syndrome, Bechcet's disease, white dot syndrome (acute posterior multifocal placoid pigment epitheliopathy, serpiginous choroiditis, birdshot chorioretinopathy, multifocal choroiditis with panuveitis, diffuse subretinal fibrosis syndrome, punctuate inner choroidopathy, multiple evanescent white dot syndrome, and diffuse unilateral subacute neuroretinitis), granuloma annulare, irritable bowel syndrome, gastroenteritis, Grave's disease, multiple sclerosis, Dupuytren's contracture, graft rejection diseases (including allograft rejection and graft-v-host disease), e.g. skin graft rejection, solid organ transplant rejection, bone marrow transplant rejection, inflammatory dermatoses.

The phrase “pharmaceutically acceptable salt(s)” as used herein refers to those salts of a compound of interest that are safe and effective for topical use in mammals and that possess the desired biological activity. Pharmaceutically acceptable salts include salts of acidic or basic groups present in the specified compounds. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds used in the present invention can form pharmaceutically acceptable salts with various amino acids. Suitable base salts include, but are not limited to, aluminum, calcium, lithium, magnesium, potassium, sodium, zinc, and diethanolamine salts. Illustrative salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like.

Illustrative organic bases are isopropylamine, diethylamine, ethanolamine, trimethylamine, dicyclohexylamine, choline, and caffeine. See Berge et al., J. Pharm. Sci. 66:1-19 (1977), incorporated herein by reference.

The term “pharmaceutically-acceptable carrier” as used herein refers to one or more compatible solid or liquid filler diluents or encapsulating substances which are suitable for administration to a subject. The term “compatible” as used herein means that the components of the composition are capable of being commingled with the active compound, and with each other, in a manner such that there is no interaction which would substantially reduce the pharmaceutical efficacy of the composition under ordinary use situations. Pharmaceutically-acceptable carriers must, of course, be of sufficiently high purity and sufficiently low toxicity to render them suitable for administration preferably to an animal, preferably mammal being treated. Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol. and polyethylene glycol; alginic acid; emulsifiers, such as the TWEEN® emulsifiers; wetting agents, such sodium lauryl sulfate; coloring agents; flavoring agents; tableting agents, stabilizers; antioxidants; preservatives; pyrogen-free water; isotonic saline; and phosphate buffer solutions. The choice of a pharmaceutically-acceptable carrier to be used in conjunction with the subject compound is basically determined by the way the compound is to be administered. If the subject compound is to be injected, the preferred pharmaceutically-acceptable carrier is sterile, physiological saline, with blood-compatible suspending agent, the pH of which has been adjusted to about 7.4. In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil. Preferably, the pharmaceutically-acceptable carrier, in compositions for parenteral administration, comprises at least about 90% by weight of the total composition. The compositions of this invention are preferably provided in unit dosage form.

The term “preventing a disease or disorder” as used herein refers to delaying the onset, hindering the progress, hindering the appearance, protection against, inhibiting or eliminating the emergence, or reducing the incidence, of such damages, effects or symptoms. Use of the term “prevention” is not meant to imply that all patients in a patient population administered a preventative therapy will never be affected by or develop symptoms in response to the disease or disorder associated with tissue damage targeted for prevention, but rather that the patient population will exhibit a reduction in the damage, effects, or symptoms of the disease or disorder.

The term “prodrug” as used herein refers to a compound that may be transformed in vivo to yield an active compound, for example, by hydrolysis, oxidaction, or other reactions, in the gut or enzymatic conversion in blood. Common examples include, but are not limited to, ester and amide forms of an active compound having an active form bearing a carboxylic acid moiety. Examples of pharmaceutically acceptable esters of the active compounds of this invention include, but are not limited to, alkyl esters (for example with between about one and about six carbons) where the alkyl group is a straight or branched chain. Acceptable esters also include cycloalkyl esters and arylalkyl esters such as, but not limited to benzyl. Examples of pharmaceutically acceptable amides of the active compounds of this invention include, but are not limited to, primary amides, and secondary and tertiary alkyl amides (for example with between about one and about six carbons). Amides and esters of the active compounds of the present invention can be prepared according to conventional methods. A thorough discussion of prodrugs is provided in Bioreversible Carriers in Drug Design, ed. Edward B. Roche, American Pharmaceutical Association and Pergamon Press, 1987 and Burger's Medicinal Chemistry and Drug Discovery, (1995) 172-178, 949-982 (Manfred E. Wolff ed., 5th ed.), which are incorporated herein by reference for all purposes.

The term “prophylactically effective amount” as used herein refers to an amount of a compound sufficient to result in the prevention of the damage, effects or symptoms resulting from a disease or disorder associated with tissue damage. A prophylactically effective amount can refer to the amount of the compound sufficient to prevent the damage, effects or symptoms resulting from a disease or disorder associated with tissue damage.

The term “radiation toxicity” or “radiation-induced toxicity” as used herein refers to radiation-induced injury to a cell or tissue arising from exposure to a radiation agent because of either radiation therapy or accidental radiation exposure. Phenotypically, radiation-induced injury includes one or more of the following: structural radiation injury to a cell or tissue, increased neutrophil infiltration, increased collagen type III deposition, and increased smooth muscle cell proliferation relative to that seen in a cell or tissue not exposed to radiation.

The term “radiation agent” as used herein refers to any radioactive material that may kill or injure a subject, and may be used for therapeutical purposes (e.g., radiotherapy) or as weapons to cause bodily injuries or harm or even death to a population. Radioactive agents may include, but are not limited to ¹³⁷Cs, ⁶⁰Co, ²⁴¹Am, ²⁵²Cf, ¹⁹²Ir, ²³⁸Pu, ⁹⁰Sr, ²²⁶Ra, ⁹¹Sr, ⁹²Sr, ⁹⁵Zr, ⁹⁹Mo, ¹⁰⁶Ru, ¹³¹Sb, ¹³²Te, ¹³⁹Te, ¹⁴⁰Ba, ¹⁴¹La, ¹⁴⁴Ce, ²³³U, ²³⁵U, ²³⁸U, ²²⁸P, ²²⁹P, ²³⁰P, ²³¹P, ²³²P, ²³³P, ²³⁴P, ²³⁵P, ²³⁶P, ²³⁷P, ²³⁸P, ²³⁹P, ²⁴⁰P, ²⁴¹P, ²⁴²P, ²⁴³P, ²⁴⁴P, ²⁴⁵P, ²⁴⁶P, ²⁴⁷P, ¹²⁴I, ¹²⁵I, ¹²⁷I, ¹³¹I, ⁹⁰Y, ¹⁶⁶Ho, ¹⁸⁶Re, ¹⁸⁸Re, ⁹⁰Sr, ²²⁶Ra, ¹⁰³Pd, ¹⁹⁸Au, ⁹⁹Tc, ¹⁸F, ²⁰¹Th, ⁶⁷Ga, and ¹¹¹In. Exposure to the radioactive agents can result in carcinogenesis, sterilization, cataract formation, radiodermatitis, beta burns, gamma burns, loss of cells (in particular bone marrow, digestive tract cells), damage to the hematopoietic, gastrointestinal, central nervous, cardiovascular, skin, and/or reproductive systems, acute radiation syndrome, chronic radiation syndrome, and cutaneous radiation syndrome. Acute radiation syndrome generally results from large doses of radiation to a subject's body occurring in a short period of time. The syndrome has a predictable course starting with a feeling of nausea, vomiting, general illness and fatigue, immune system depression, loss of hair, uncontrollable bleeding (mouth, under the skin, kidneys), massive diarrhea, delirium, coma and death. Cutaneous radiation syndrome is a subset of acute radiation syndrome and refers to radiations effects on the skin, which include, but are not limited to, inflammation, erythema, dry or moist desquamation, hair loss, blistering, reddening, ulceration, damage to sebaceous and sweat glands, atrophy, fibrosis, decreased or increased skin pigmentation, and necrosis.

The terms “systemic administration” and “systemically administered” as used herein refer to a method of administering a compound or a pharmaceutical composition to a subject so that the compound or pharmaceutical composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “therapeutically effective amount” of a compound as used herein refers to a sufficient amount of the compound to treat a disease or disorder, at a reasonable benefit/risk ratio applicable to any medical treatment. It will be understood, however, that the total daily usage of the compound will be decided by the attending physician within the scope of sound medical judgment. The specific therapeutically effective dose level for any particular patient will depend upon a variety of factors including the disorder being treated and the severity of the disorder; activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific antibody employed; and like factors well known in the medical arts. For example, it is well known within the skill of the art to start doses of the compound at levels lower than those required to achieve the desired therapeutic effect and to gradually increase the dosage until the desired effect is achieved.

The terms “tissue damage” and “tissue injury” are used herein interchangeably, which refer to any damage of a tissue that disrupts its physical structure resulting in the impairment of its function. For example, tissue injury may be caused by any form of chemical or physical agents, such as, drugs, environmental toxicants, or any other substance that contacts a subject and results directly or indirectly, in damage to the cells of the organ or tissue. Also included, is cellular damage that results from successful therapeutic treatment of a subject, such as for example, the treatment of a tumor which results in induction of apoptosis. Similarly, tissue injury might be the result of a physical agent such as, for example, exposure to an environmental condition such as a hypoxic condition or air or water pollution. Alternatively, the physical agent might be a physical trauma event, whether self-imposed or not, such as for example, exercise, smoking, blunt force trauma, stroke, etc.

The term “tissue protective activity” or “tissue protection” as used herein refers to the effect of inhibiting or delaying damage or death of a cell, tissue, or organ. Unless otherwise noted, the “delay” in damage or death of a cell, tissue or organ is evaluated relative to a control condition in the absence of a compound of the invention. Tissue protective activity is specific to tissue, cells, and/or organs expressing a tissue protective receptor complex (i.e., a responsive tissue cell, and/or organ, respectively), such as, but not limited to, the tissues of the central nervous system. In specific embodiments, the responsive cells are not erythrocyte progenitor cells.

The term “toxic agent” as used herein refers to a chemical agent or a radiation agent disclosed herein. The term “chemical agent” as used herein refers a chemical substance that is administered to a subject for therapeutical purposes or used as chemical weapon to cause severe injuries or harm to the subject. Therapeutical chemical agent includes many pharmaceutical compounds that may also cause toxicity to the subject (i.e., side effects). The chemical agents used as weapons can be classified by their method of action such as: blood agents, blister agents, nerve agents, pulmonary agents, and incapacitating agents.

The term “treatment,” “treat,” or “treating” as used herein refers to clinical intervention in an attempt to alter the natural course of the individual being treated, and can be performed either for prophylaxis or during the course of clinical pathology. For purposes of this invention, beneficial or desired clinical results include, but are not limited to, alleviation of symptoms; diminishment of extent of disorder or disease; stabilized (i.e. not worsening) state of disorder or disease; delay or slowing of disorder, or disease progression; amelioration of the disorder or disease state, remission (whether partial or total), whether detectable or undetectable; or enhancement or improvement of the disorder or disease. Treatment includes eliciting a cellular response that is clinically significant, without excessive levels of side effects. Treatment also includes prolonging survival as compared to expected survival if not receiving treatment.

### DETAILED DESCRIPTION OF PREFERRED EMBODIMENTS

The description that follows includes systems, methods, techniques, instruction sequences, and computing machine program products that embody illustrative embodiments of the present invention. In the following description, for purposes of explanation, numerous specific details are set forth in order to provide an understanding of various embodiments, it will be evident, however, to those skilled in the art that embodiments of the inventive subject matter may be practiced without these specific details. In general, well-known instruction instances, protocols, structures, and techniques have not been shown in detail.

Additionally, the terminology used herein is for the purpose of description and not for limitation. Furthermore, although certain methods are described with reference to steps that are presented herein in a certain order, in many instances, these steps can be performed in any order as may be appreciated by one skilled in the art; the novel method is therefore not limited to the particular arrangement of steps disclosed herein. It is to be understood that each component, compound, substituent, or parameter disclosed herein is to be interpreted as being disclosed for use alone or in combination with one or more of each and every other component, compound, substituent, or parameter disclosed herein.

It must be noted that as used herein and in the appended claims, the singular forms “a”, “an”, and “the” include plural references unless the context clearly dictates otherwise. Furthermore, the terms “a” (or “an”), “one or more”, and “at least one” can be used interchangeably herein. The terms “comprising”, “including”, “having” and “constructed from” can also be used interchangeably.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure.

The present invention provides a method for preventing or treating a disease or disorder associated with tissue damages using an inhibitor of MST1/2 protein kinases, its prodrugs, or a pharmaceutical composition comprising the inhibitor of MST1/2 protein kinases.

The inhibitors of MST1/2 protein kinases, their prodrugs and pharmaceutical compositions, are described previously in PCT publication WO 2017/148406 A1, which are incorporated herein by reference in its entirety. Briefly, the inhibotrs of MST1/2 protein kinases may be represented by the following general formula:

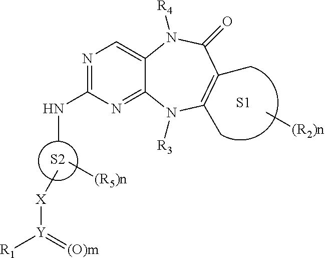

wherein R₁ is selected:
1) C1-C6 alkyl, optionally substituted by halogen, nitro, cyano; C1-C6 alkyl group containing oxygen; C3-C7 cycloalkyl, which is optionally substituted by halogen, nitro, cyano; —O—C1-C6 alkyl, optionally substituted by halogen, nitro, amino, cyano; —O-C3-C7 cycloalkyl, which is optionally substituted with halogen, nitro, amino, cyano; C6-C10 aryl, which is optionally substituted by halogen, nitro, amino, cyano; —O-C6-C10 aryl, which is optionally substituted by halogen, nitro, amino, cyano; C2-C6 alkenyl group;
2) 3-N, N-dimethylamino-propenyl, 3-pyrrolidin-propenyl;
3) amino, cyclopropylamino, cyclobutylamino, cyclopentylamino, cyclohexylamino, N, N-dimethylamino, N, N-diethylamino, N, N-diisopropylamino, 2-N, N-dimethyl-ehthylamino, 2-hydroxyethylamino, 2-morpholinyl-ethylamino, 2-thiomorpholinyl ethylamino, 2-(4-N-methyl piperazinyl) ethylamino, 3-N, N-dimethyl-aminopropyl amino, 3-N, N-diethyl aminopropyl amino, 3-N, N-diisopropyl-aminopropyl amino, amino-3-hydroxylpropyl, 3-morpholinyl-propylamino, 3-thiomorpholinyl propylamino, 3-(4-N-methylpiperazinyl) propylamino, N-methylpiperidinyl-4-amino, N-ethylpiperidiny-4-amino, N-isopropyl-piperidinyl-4-amino, N-acetyl-piperidinyl-4-amino; 4) hydroxyl, 2-N,N-dimethylaminoethoxyl, 2-N, N-diethyl-aminoethoxyl, 2-N, N-diisopropyl-aminoethoxyl, 2-(N-methylpiperazinyl) ethoxyl, 2-(N-acetyl-piperazinyl) ethoxyl, 2-morpholinyl-ethoxyl, 2-thiomorpholinyl ethoxyl, 2-piperidinyl-ethoxyl, 3-N, N-dimethylamino-propoxyl, 3-N, N-diethylamino-propoxyl, 3-N, N-diisopropylamino propoxyl, 3-(N-methylpiperazinyl) propoxyl, 3-(N-acetyl-piperazinyl) propoxyl, 3-morpholinyl-propoxyl, 3-thiomorpholinyl propoxyl, 3-piperidinyl-propoxyl, 2-pyridyl-methoxyl, 3-pyridyl-methoxyl, 4-pyridyl methoxyl, phenylmethoxyl, monohalogen-substituted phenylmethoxyl, homodihalogen-substituted phenylmethoxyl, heterodihalogen-substituted phenylmethoxyl;
5) selected from the group of five- or six-membered heterocyclic rings comprising one or a more of N, S and O heteroatoms, said five- or six-membered heterocyclic rings are optionally substituted with C1-C6 alkyl, C1-C6 alkoxy, hydroxy, amino, C1-C6 acyl, cyano, or heterocyclic group,
including but not limited to: piperidinyl, 4-N, N-dimethylamino-piperidinyl, 4-N, N-diethylamino-piperidinyl, 4-N, N-diisopropylamino piperidinyl, 4-hydroxypiperidinyl, 4-(N-methylpiperazinyl) piperidinyl, 4-(N-ethyl-piperazinyl) piperidinyl, 4-(N-isopropyl-piperazinyl) piperidinyl, 4-(N-acetyl-piperazinyl) piperidinyl, 4-(N-tert-butoxyl formyl-piperazinyl) piperidinyl, 4-(N-methylsulfonyl-piperazinyl) piperidinyl, 4-(N-(2-hydroxylethyl) piperazinyl) piperidinyl, 4-(N-(2-cyanoethyl) piperazinyl) piperidinyl, 4-(N-(3-hydroxylpropyl) piperazinyl) piperidinyl, 4-(N-(2-N, N-dimethyl-ethyl) piperazinyl) piperidinyl, 4-(N-(2-N, N-diethyl-ethyl) piperazinyl) piperidinyl, 4-(N-(3-N, N-dimethyl-propyl) piperazinyl) piperidinyl, 4-(N-(3-N,N-diethyl-propyl) piperazinyl) piperidinyl, 4-(pyrrolidinyl) piperidinyl, 4-(3-N, N-dimethyl-pyrrolidinyl) piperidinyl;
N-methyl-piperazinyl, N-ethyl-piperazinyl, N-isopropyl-piperazinyl, N-acetyl-piperazinyl, N-tert formyl piperazinyl, N-methylsulfonyl-piperazinyl piperazinyl, N-(2-hydroxylethyl) piperazinyl, N-(2-cyanoethyl) piperazinyl, N-(3-hydroxylpropyl) piperazinyl, N-(2-N, N-dimethylethyl) piperazinyl, N-(2-N, N-diethyl-ethyl) piperazinyl, N-(3-N, N-dimethyl-propyl) piperazinyl, N-(3-N, N-diethyl-propyl) piperazinyl, 2-oxo-piperazin-4-yl, N—(N-methyl-4-piperidinyl) piperazinyl, N—(N-ethyl-4-piperidinyl) piperazinyl, N—(N-acetyl-4-piperidinyl) piperazinyl;
morpholinyl, 3,5-dimethyl morpholinyl, thiomorpholinyl, tetrahydropyrrolyl, 3-N, N-dimethyl-tetrahydropyrrolyl, 3-N, N-diethyl-tetrahydropyrrolyl;
R₂ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamino, ethylsulfonamino, propylsulfonamido, isopropylsulfonamido, dimethyl phosphinyl, diethyl phosphinyl, diisopropyl phosphinyl;
R₃ is selected from: hydrogen; C1-C6 alkyl, optionally substituted by halogen, nitro, amino, cyano; C3-C7 cycloalkyl, which is optionally substituted with halogen, nitro, amino, cyano; alternatively, R₃ may form a five-member ring with its connected N atom and C atom in the S1 ring;
R₄ is selected from: hydrogen; C1-C6 alkyl, optionally substituted by halogen, nitro, amino, cyano;
C3-C7 cycloalkyl, which is optionally substituted with halogen, nitro, amino, cyano;
R₅ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
X=O, NH or a direct bond;
Y=S, C, P, N, OH, NH₂ or CH₂;
m=0, 1 or 2;
n=0, 1, 2, 3 or 4;

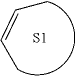

is aryl or heteroaryl group fused with a seven membered two-nitrogen heterocyclic ring, such as benzene ring, a thiophene ring, a furan ring, a pyridine ring, an oxazole ring, or thiazolyl ring fused with a seven membered two nitrogen heterocyclic ring group;

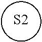

is aryl or heteroaryl, such as a benzene ring or a pyrazole ring;
or a stereoisomer of the above compounds, a prodrug thereof, a pharmaceutically acceptable salt thereof, or a pharmaceutically acceptable solvate thereof.

A second aspect, the inhibitors of MST1/2 protein kinases have the following formulas I, II, III, IV:

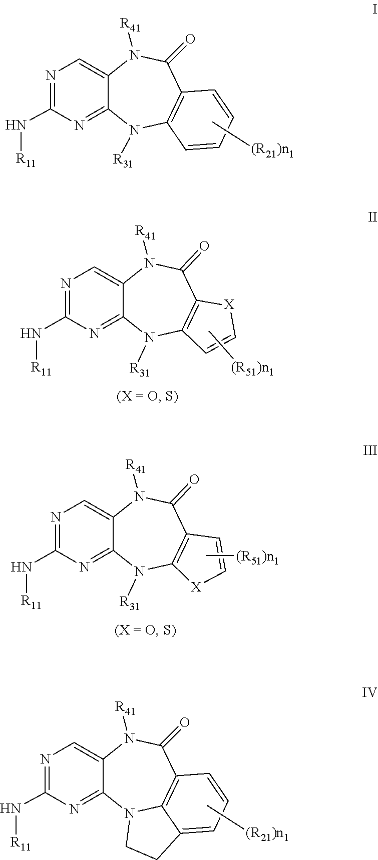

wherein: n1 is selected from 0, 1, 2, 3 or 4;
R₁₁ is selected from:
1) C1-C6 alkyl, optionally substituted with halogen, amino, nitro, cyano; C1-C6 alkyl containing oxygen; C3-C7 cycloalkyl, which is optionally substituted with halogen, amino, nitro, cyano; C6-C10 aryl, optionally substituted by halogen, nitro, amino, hydroxy, cyano; C3-C6 alkenyl;
2) 2-N, N-dimethylaminoethyl, 2-hydroxyethyl, 2-N, N-diethylaminoethyl, 2-N, N-diisopropylamino ethyl, 2-morpholinyl ethyl, 2-thiomorpholinyl ethyl, 2-(4-N-piperazinyl-methyl) ethyl, 3-N, N-dimethylaminopropyl, 3-N, N-diethylaminopropyl, 3-N, N-diisopropyl-aminopropyl, 3-morpholinyl propyl, 3-thiomorpholinyl propyl, 3-(4-N-methylpiperidinyl) propyl, 4-N, N-dimethylamino-cyclohexyl, 4-N, N-diethylamino cyclohexyl, N-methyl-4-piperidinyl, N-ethyl-4-piperidinyl, N-isopropyl-4-piperidinyl, 1,3-dimethyl-5-pyrazolyl, 1-methyl-4-pyrazolyl, 3-methyl-5-isoxazolinyl, 1-(N-methyl-4-piperidinyl)-4-pyrazolyl, 1-(N-tert-butoxyl formyl-4-piperidinyl)-4-pyrazolyl; 3)

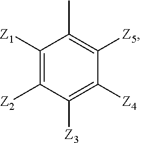

wherein Z₁, Z₂, Z₃, Z₄, Z₅ are each independently selected from:
(1) hydrogen, halogen, nitro, amino, hydroxy, cyano,
(2) C1-C6 alkyl, C1-C6 alkoxy, C1-C6 alkyl containing oxygen, C1-C6 alkyl containing fluorine, C1-C6 alkoxy containing fluorine, 4-piperidinyl, N-methyl yl-4-piperidinyl,
(3) N, N-dimethylamino, N, N-diethylamino, N, N-diisopropylamino, 2-N, N-dimethylaminoethylamino, 2-morpholino ethylamino, 2-ethylamino thiomorpholinyl, 2-(4-N-methylpiperazinyl) ethylamino, 3-N, N-dimethyl-aminopropyl amino, 3-N, N-diethylaminopropyl-amino, 3-N, N-diisopropylamino propylamino, 3-morpholin-propylamino, 3-thiomorpholinyl propylamino, 3-(4 N-methylpiperazinyl) propylamino, N-methylpiperidinyl-4-amino, N-ethylpiperidinyl-4-amino, N-isopropyl-piperidinyl-4-amino,
(4) 2-N, N-dimethylaminoethoxyl, 2-N, N-diethyl-aminoethoxy, 2-N, N-diisopropyl-aminoethoxyl, 2-(N-methylpiperazinyl) ethoxyl, 2-(N-acetyl-piperazinyl) ethoxyl, 2-morpholino-ethoxyl, 2-thiomorpholino-ethoxyl, 2-piperidinyl ethoxyl, 3-N, N-dimethylamino-propoxyl, 3-N, N-diethylamino-propoxyl, 3-N, N-diisopropylamino propoxyl, 3-(N-methylpiperazinyl) propoxyl, 3-(N-acetyl-piperazinyl) propoxyl, 3-morpholinyl-propoxyl, 3-thiomorpholinyl propoxyl, 3-piperidinyl-propoxyl, 2-pyridyl-methoxyl, 3-pyridyl-methoxyl, 4-pyridyl methoxyl, phenylmethoxyl, monohalogen-substituted phenylmethoxyl, homodihalogen-substituted phenylmethoxyl, heterodihalogen-substituted phenylmethoxyl,
(5) piperidinyl, 4-N, N-dimethylamino-piperidinyl, 4-N, N-diethylamino-piperidinyl, 4-N, N-diisopropylamino piperidinyl, 4-hydroxy piperidinyl, morpholinyl, 3,5-dimethyl morpholinyl, thiomorpholinyl, tetrahydropyrrolyl, 3-N, N-dimethyl-tetrahydropyrrolyl, 3-N, N-diethyl-tetrahydropyrrolyl, N-methyl-piperazinyl, N-ethyl-piperazinyl, N-isopropyl-piperazinyl, N-acetyl-piperazinyl, N-tert-butoxyl formyl piperazinyl, N-methylsulfonyl-piperazinyl, N-(2-hydroxylethyl) piperazinyl, N-(2-cyanoethyl) piperazinyl, N-(3-hydroxylpropyl) piperazinyl, N-(2-N, N-dimethylethyl) piperazinyl, N-(2-N, N-diethyl-ethyl) piperazinyl, N-(3-N, N-dimethylpropyl) piperazinyl, N-(3-N, N-diethyl-propyl) piperazinyl, 2-oxo-piperazinyl, 2-oxo-piperazin-4-yl, imidazolyl, 4-imidazolyl,
(6) 4-(N-methylpiperazinyl) piperidinyl, 4-(N-ethyl-piperazinyl) piperidinyl, 4-(N-isopropyl-piperazinyl) piperidinyl, 4-(N-acetyl-piperazinyl) piperidinyl, 4-(N-t-butoxyl-formyl-piperazinyl) piperidinyl, 4-(N-methylsulfonyl-piperazinyl) piperidinyl, 4-(N-(2-hydroxylethyl) piperazinyl) piperidinyl, 4-(N-(2-cyanoethyl) piperazinyl) piperidinyl, 4-(N-(3-hydroxylpropyl) piperazinyl) piperidinyl, 4-(N-(2-N, N-dimethyl-ethyl) piperazinyl) piperidinyl, 4-(N-(2-N, N-diethyl ethyl) piperazinyl) piperidinyl, 4-(N-(3-N, N-dimethyl-propyl) piperazinyl) piperidinyl, 4-(N-(3-N, N-diethyl-propyl) piperazinyl) piperidinyl, 4-(tetrahydropyrrolyl) piperidinyl, 4-(3-N, N-dimethyl-tetrahydropyrrolyl) piperidinyl, N—(N-methyl-4-piperidinyl) piperazinyl, N—(N-ethyl-4-piperidinyl) piperazinyl,
(7) hydroxy sulfonyl, aminosulfonyl, sulfonyl methylamino, ethylamino sulfonyl group, a sulfonyl group propylamino, isopropylamino-sulfonyl, aminosulfonyl cyclopropyl, cyclobutyl aminosulfonyl, cyclopentyl aminosulfonyl, piperidinyl-sulfonyl, 4-hydroxyl-piperidinyl-1-sulfonyl, 4-N, N-dimethyl-piperidinyl-1-sulfonyl, 4-N, N-diethyl-piperidinyl-1-sulfonyl, pyrrolidinyl-1-sulfonyl, 3-N, N-dimethyl-pyrrolidinyl-1-sulfonyl, 3-N, N-diethyl-pyrrolidinyl-1-sulfonyl, N-methyl-piperazinyl-sulfonyl, N-ethylpiperazinyl-1-sulfonyl, N-acetyl-piperazinyl-1-sulfonyl, N-tert-butoxylformyl-piperazinyl-1-sulfonyl, N-(2-hydroxylethyl) piperazinyl-1-sulfonyl, N-(2-cyanoethyl) piperazinyl-1-sulfonyl, N-(2-N, N-dimethyl ethyl) piperazinyl-1-sulfonyl, N-(2-N, N-diethyl-ethyl) piperazinyl-1-sulfonyl, N-(3-hydroxylpropyl) piperazinyl-1-sulfonyl, N-(3-N, N-dimethylamino-propyl) piperazinyl-1-sulfonyl, N-(3-N, N-diethylamino-propyl) piperazinyl-1-sulfonyl, morpholinyl-1-sulfonyl, 3,5-dimethyl-morpholinyl-1-sulfonyl, 4-(N-methyl-1-piperazinyl) piperidinyl-1-sulfonyl, 4-(N-ethyl-1-piperazinyl) piperidinyl-1-sulfonyl, 4-(N-acetyl-1-piperazinyl) piperidinyl-sulfonyl, N—(N-methyl-4-piperidinyl) piperazinyl-1-sulfonyl,
(8) amino formyl, methylamino formyl, ethylamino formyl, propylamino formyl, isopropylamino formyl, cyclopropylamino formyl, cyclobutylamino formyl, cyclopentylamino formyl, piperidinyl-1-formyl, 4-hydroxy-piperidinyl-1-formyl, 4-N, N-dimethyl-piperidinyl-1-formyl, 4-N, N-two ethylpiperidinyl-1-formyl, tetrahydropyrrolyl-1-formyl, 3-N, N-dimethyl-tetrahydropyrrolyl-1-formyl, 3-N, N-diethyl-tetrahydropyrrolyl-1-formyl, N-methyl-piperazinyl-1-formyl, N-ethyl-piperazinyl-1-formyl, N-acetyl-piperazinyl-1-formyl, N-tert-butoxyl-formyl-piperazinyl-1-formyl, N-(2-hydroxyethyl) piperazinyl-1-formyl, N-(2-cyanoethyl) piperazinyl-1-formyl, N-(2-N, N-dimethyl-ethyl) piperazinyl-1-formyl, N-(2-N, N-diethyl-ethyl) piperazinyl-1-formyl, N-(3-hydroxypropyl) piperazinyl-1-formyl, N-(3-N, N-dimethyl-propyl) piperazinyl-1-formyl, N-(3-N, N-diethyl propyl) piperazinyl-1-formyl, morpholinyl-1-formyl, 3,5-dimethyl-morpholinyl-1-formyl, 4-(N-methyl-1-piperazinyl) piperidinyl-1-formyl, 4-(N-ethyl-1-piperazinyl) piperidinyl-1-formyl, 4-(N-acetyl-1-piperazinyl) piperidinyl-1-formyl, N—(N-methyl-4-piperidinyl) piperazinyl-1-formyl,
(9) hydroxyl formyl, methoxyl formyl, ethoxyl formyl, propoxyl formyl, isopropoxyl formyl, n-butoxyl formyl, isobutoxy formyl, t-butoxyl formyl,
(10) amino formamido, methylamino formamido, ethylamino formamido, propylamino formamido, isopropylamino formamido, cyclopropylamino formamido, cyclobutylamino formamido, cyclopentylamino formamido, piperidinyl-1-formamido, 4-hydroxy-piperidinyl-1-formamido, 4-N, N-dimethyl-piperidinyl-1-formamido, 4-N, N-diethyl-piperidinyl-1-formamido, tetrahydropyrrolyl-1-formamido, 3-N, N-dimethyl-tetrahydropyrrolyl-1-formamido, 3-N, N-diethyl-tetrahydropyrrolyl-1-formamido, N-methyl-piperazinyl-1-formamido, N-ethyl-piperazinyl-1-formamido, N-acetyl-piperazinyl-1-formamido, N-tert-butoxyl formyl-piperazinyl-1-formamido, N-(2-hydroxyethyl) piperazinyl-1-formamido, N-(2-cyanoethyl) piperazinyl-1-formamido, N-(2-N, N-dimethyl-ethyl) piperazinyl-1-formamido, N-(2-N, N-diethyl-ethyl) piperazinyl-1-formamido, N-(3-hydroxypropyl) piperazinyl-1-formamido, N-(3-N, N-dimethyl-propyl) piperazinyl-1-formamido, N-(3-N, N-diethyl-aminopropyl) piperazinyl-1-formamido, morpholinyl-1-formamido, 3,5-dimethyl-morpholinyl-1-formamido, 4-(N-methyl-1-piperazinyl) piperidinyl-1-formamido, 4-(N-ethyl-1-piperazinyl) piperidinyl-1-formamido, 4-(N-acetyl-1-piperazinyl) piperidinyl-1-formamido, N—(N-methyl-4-piperidinyl) piperazinyl-1-formamido; or
(11) amino acetamido, N-tert-butoxyl formyl acetamido, N-acetylamino acetamido, acrylamido, cyclopropylamido, chloroacetamido, bromoacetamido, piperidinyl acetamido, 4-hydroxy piperidinyl acetamido, 4-N, N-dimethyl-piperidinyl-acetamido, 4-N, N-diethyl-piperidinyl acetamido, tetrahydropyrrolyl acetamido, 3-N, N-dimethyl-tetrahydropyrrolyl acetamido, 3-N, N-diethyl-tetrahydropyrrolyl-acetamido, N-methyl-piperazinyl acetamido, N-ethyl piperazinyl-acetamido, N-acetyl-piperazinyl acetamido, N-tert-butoxy formyl-piperazinyl acetamido, N-(2-hydroxyethyl) piperazinyl acetamido, N-(2-cyanoethyl) piperazinyl acetamido, N-(2-N, N-dimethylethyl) piperazinyl acetamido, N-(2-N, N-diethyl-ethyl) piperazinyl acetamido, N-(3-hydroxylpropyl) piperazinyl acetamido, N-(3-N, N-dimethyl-propyl) piperazinyl acetamido, N-(3-N, N-diethyl-propyl) piperazinyl acetamido, morpholinyl acetamido, 3,5-dimethyl-morpholinyl-acetamido, 4-(N-methyl-1-piperazinyl) piperidinyl acetamido, 4-(N-ethyl-1-piperazinyl) piperidinyl acetamido, 4-(N-acetyl-1-piperazinyl) piperidinyl acetamido, N—(N-methyl-4-piperidinyl) piperazinyl acetamido, 4-(tetrahydropyrrolyl) piperidinyl acetamido; 2-methylamino acetamido, 2-(1-methylethyl) amino acetamido; N-benzyloxy-formyl-2-methylamino-acetamido;
(12) Z₂ and Z₃ may form a substituted or unsubstituted oxygen-containing five- or six-membered ring; the substituents may be selected from the same substituents of Z₁,
(13) Z₂ and Z₃ may form a substituted or unsubstituted nitrogen-containing five- or six-membered ring; the substituents may be selected from the same substituents of Z₁,
4)

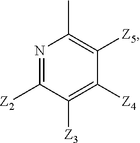

wherein Z₂, Z₃, Z₄, Z₅ are the same as the definition 3) above;
5)

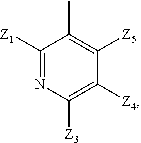

wherein Z₁, Z₃, Z₄, Z₅ are the same as the definition 3) above;
R₂₁ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; a C1-C6 oxygen-containing alkyl;
3) methylthio, ethylthio, isopropylthio, methylsulfinyl, ethylsulfinyl, propyl sulfinyl, methylsulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
R₃₁ is selected from:
Hydrogen; C1-C6 alkyl, optionally substituted by halogen, nitro, amino, cyano; C3-C7 cycloalkyl, which is optionally substituted by halogen, nitro, amino, cyano;
R₁₁ is selected from:
hydrogen; C1-C6 alkyl, optionally substituted by halogen, nitro, amino, cyano; C3-C7 cycloalkyl, which is optionally substituted by halogen, nitro, amino, cyano;
R₅₁ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
or a stereoisomer of the above compounds, a prodrug thereof, a pharmaceutically acceptable salt thereof, or a pharmaceutically acceptable solvate thereof.

A third aspect, the inhibitors of MST1/2 protein kinases are represented by the following structural formula:

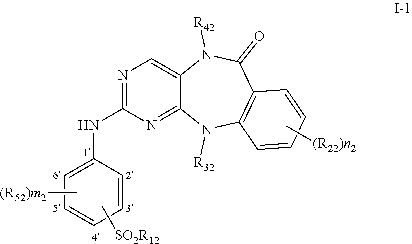

wherein m2 is selected from 0, 1, 2, 3 or 4;
n2 is selected from 0, 1, 2, 3 or 4;
R₁₂ is selected from:
1) selected from the group of five membered or six-membered heterocyclic rings comprising one or more N, O and S heteroatoms, the five membered or six-membered heterocyclic rings are optionally substituted with C1-C6 alkyl, C1-C6 alkoxy, hydroxy, amino, alkylamino, dialkylamino, C1-C6 acyl, cyano, optionally substituted C1-C6 alkyl, —O-C1-C6 alkyl, hydroxy, hydroxy C1-C6 alkyl, C1-C6 acyl, alkylamino, dialkylamino substituted heterocyclic group,
including but not limited to: 4-N, N-dimethylamino-piperidinyl, 4-N, N-diethylamino-piperidinyl, 4-N, N-diisopropylamino-piperidinyl, 4-hydroxy-piperidinyl, 4-(N-methylpiperazinyl) piperidinyl, 4-(N-ethyl-piperazinyl) piperidinyl, 4-(N-isopropyl-piperazinyl) piperidinyl, 4-(N-acetyl-piperazinyl) piperidinyl, 4-(N-tert-butoxyl formyl-piperazinyl) piperidinyl, 4-(N-methylsulfonyl-piperazinyl) piperidinyl, 4-(N-(2-hydroxylethyl) piperazinyl) piperidinyl, 4-(N-(2-cyanoethyl) piperazinyl) piperidinyl, 4-(N-(3-hydroxylpropyl) piperazinyl) piperidinyl, 4-(N-(2-N, N-dimethyl-ethyl) piperazinyl) piperidinyl, 4-(N-(2-N, N-diethyl-ethyl) piperazinyl) piperidinyl, 4-(N-(3-N, N-dimethyl-propyl) piperazinyl) piperidinyl, 4-(N-(3-N, N-diethyl-propyl) piperazinyl) piperidinyl, 4-(tetrahydropyrrolyl) piperidinyl, 4-(3-N, N-dimethyl-tetrahydropyrrolyl) piperidinyl; 2) amino, cyclopropylamino, cyclobutylamino, cyclopentylamino, cyclohexylamino, N, N-dimethylamino, N, N-diethylamino, N, N-diisopropylamino, 2-N, N-dimethylaminoethylamino, 2-hydroxyethylamino, 2-morpholinyl-ethylamino, 2-(4-N-methylpiperazinyl) ethylamino, 3-N, N-dimethylamino-propylamino, 3-N, N-diethylamino-propylamino, 3-N, N-diisopropylamino-propylamino, 3-hydroxy-propylamino, 3-morpholinyl-propylamino, 3-(4-N-methylpiperazinyl) propylamino, N-methylpiperidinyl-4-amino, N-ethylpiperidinyl-4-amino, N-isopropyl piperidinyl-4-amino, N-acetyl piperidinyl-4-amino; N-methyl-piperazinyl, N-ethyl-piperazinyl, N-isopropyl-piperazinyl, N-acetyl piperazinyl, N-tert-butoxyl formyl-piperazinyl, N-methylsulfonyl-piperazinyl, N-(2-hydroxylethyl) piperazinyl, N-(2-cyanoethyl) piperazinyl, N-(3-hydroxylpropyl) piperazinyl, N-(2-N, N-dimethylethyl) piperazinyl, N-(2-N, N-diethyl aminoethyl) piperazinyl, N-(3-N, N-dimethyl-propyl) piperazinyl, N-(3-N, N-diethyl-propyl) piperazinyl, 2-oxo-piperazin-4-yl, N—(N-Methyl-4-piperidinyl) piperazinyl, N—(N-ethyl-4-piperidinyl) piperazinyl, N—(N-acetyl-4-piperidinyl) piperazinyl; morpholinyl, 3,5-dimethyl morpholinyl, thiomorpholinyl, tetrahydropyrrolyl, 3-N, N-dimethyl-tetrahydropyrrolyl, 3-N, N-diethyl-tetrahydropyrrolyl;
3) C1-C6 alkyl, optionally substituted by halogen, nitro, cyano;
4) C3-C7 cycloalkyl, which is optionally substituted by halogen, nitro, cyano;
5) —O—C1-C6 alkyl, optionally substituted by halogen, nitro, amino, cyano;
6) —O—C3-C7 cycloalkyl, which is optionally substituted by halogen, nitro, amino, cyano;
7) C6-C10 aryl, which is optionally substituted by halogen, nitro, amino, cyano; —O-C6-C10 aryl group, which is optionally substituted by halogen, nitro, amino, cyano;
8) C2-C6 alkenyl;
9) hydroxyl, 2-N, N-dimethylaminoethoxyl, 2-N, N-diethyl-aminoethoxyl, 2-N, N-diisopropyl-aminoethoxyl, 2-(N-methylpiperazine-yl) ethoxyl, 2-(N-acetyl-piperazinyl) ethoxyl, 2-morpholinyl-ethoxyl, 2-thiomorpholinyl ethoxyl, 2-piperidinyl-ethoxyl, 3-N, N-dimethylamino-propoxyl, 3-N, N-diethylamino-propoxyl, 3-N, N-diisopropylamino propoxyl, 3-(N-methylpiperazinyl) propoxyl, 3-(N-acetyl-piperazinyl) propoxyl, 3-morpholinyl-propoxyl, 3-thiomorpholinyl propoxyl, 3-piperidinyl-propoxyl, 2-pyridyl-methoxyl, 3-pyridyl-methoxyl, 4-pyridyl methoxyl, phenylmethoxyl, monohalogen-substituted phenylmethoxyl, homodihalogen substituted phenyl methoxyl, heterodihalogen-substituted phenylmethoxyl;
R₂₂ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamiso, ethylsulfonamiso, propylsulfonamiso, isopropylsulfonamiso, dimethyl phosphinyl, diethyl phosphinyl, diisopropyl phosphinyl;
R₃₂ is selected from: hydrogen; C1-C6 alkyl, optionally substituted by halogen, nitro, amino, cyano; C3-C7 cycloalkyl, which is optionally substituted with halogen, nitro, amino, cyano; R₄₂ is selected from: hydrogen; C1-C6 alkyl, optionally substituted by halogen, nitro, amino, cyano; C3-C7 cycloalkyl, which is optionally substituted with halogen, nitro, amino, cyano;
R₅₂ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino-sulfonyl, cyclopropyl aminosulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
Or a stereoisomer of the above compounds, a prodrug thereof, a pharmaceutically acceptable salt thereof, or a pharmaceutically acceptable solvate thereof.

A fourth aspect, the inhibitors of MST1/2 protein kinases are represented by the following structural formula:

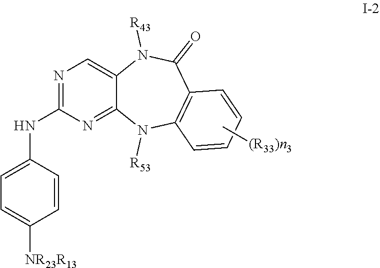

wherein n3 is selected from 0, 1, 2, 3 or 4;
R₂₃ is —S—O₂X, wherein X is selected from: hydroxy; C1-C6 alkyl, optionally substituted by halogen, nitro, amino, cyano; C3-C7 cycloalkyl, which is optionally substituted with halogen, nitro, amino, cyano;
R₁₃ is selected from: hydrogen; C1-C6 alkyl, optionally substituted by halogen, nitro, amino, cyano; C3-C7 cycloalkyl, which is optionally substituted with halogen, nitro, amino, cyano;
R₃₃ is selected from:
1) a hydrogen, halo, nitro, amino, cyano;
2) C1-C6 alkyl, optionally substituted by halogen, nitro, amino, cyano; —O—C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropyl aminoformyl, cyclobutyl aminoformyl, cyclopentylaminoformyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
R₅₃ is selected from: hydrogen; C1-C6 alkyl, optionally substituted by halogen, nitro, amino, cyano; C3-C7 cycloalkyl, which is optionally substituted with halogen, nitro, amino, cyano;
R₄₃ is selected from: hydrogen; C1-C6 alkyl, optionally substituted by halogen, nitro, amino, cyano; C3-C7 cycloalkyl, which is optionally substituted with halogen, nitro, amino, cyano;
Or a stereoisomer of the above compounds, a prodrug thereof, a pharmaceutically acceptable salt thereof, or a pharmaceutically acceptable solvate thereof.

MST1/2 protein kinase inhibitors having Formula I are exemplified by the compounds in Table 1.

TABLE 1
MST1/2 protein kinase inhibitors having Formula I

| | |
|---|---|
| 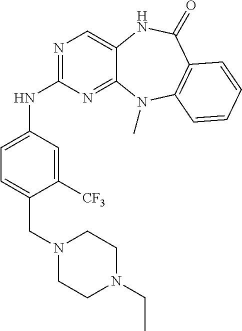 [SMILES: CCN1CCN(Cc2ccc(Nc3ncc4c(n3)N(C)c3ccccc3C(=O)N4)cc2C(F)(F)F)CC1] | IA-1 |
| (TFA Salt) | |
| 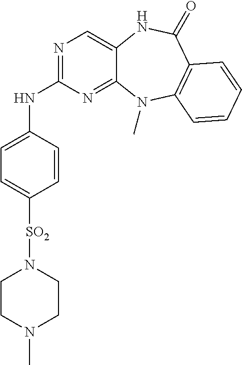 [SMILES: CN1CCN(S(=O)(=O)c2ccc(Nc3ncc4c(n3)N(C)c3ccccc3C(=O)N4)cc2)CC1] | IA-2 |
| 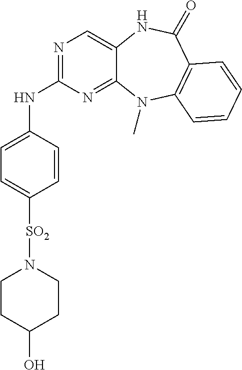 [SMILES: CN1c2ccccc2C(=O)Nc2cnc(Nc3ccc(S(=O)(=O)N4CCC(O)CC4)cc3)nc21] | IA-3 |
| 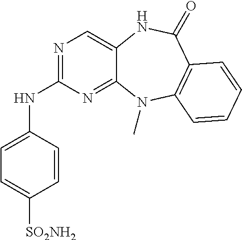 [SMILES: CN1c2ccccc2C(=O)Nc2cnc(Nc3ccc(S(N)(=O)=O)cc3)nc21] | IA-4 |
| (TFA Salt) | |
| 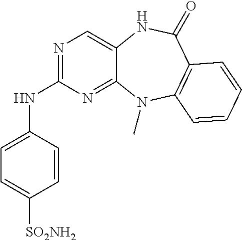 [SMILES: CN1c2ccccc2C(=O)Nc2cnc(Nc3ccc(S(N)(=O)=O)cc3)nc21] | IA-5 |
| (HCl Salt) | |
| 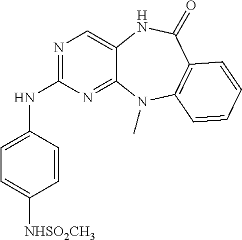 | IA-6 |
| (HCl Salt) | |
| 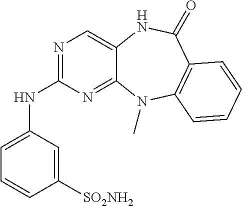 [SMILES: CN1c2ccccc2C(=O)Nc2cnc(Nc3cccc(S(N)(=O)=O)c3)nc21] | IA-7 |
| (HCl Salt) | |
| 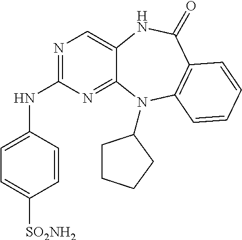 [SMILES: NS(=O)(=O)c1ccc(Nc2ncc3c(n2)N(C2CCCC2)c2ccccc2C(=O)N3)cc1] | IB-1 |
| (TFA Salt) | |
| 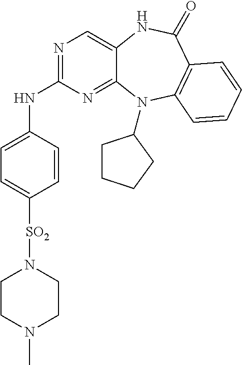 [SMILES: CN1CCN(S(=O)(=O)c2ccc(Nc3ncc4c(n3)N(C3CCCC3)c3ccccc3C(=O)N4)cc2)CC1] | IB-2 |
| (TFA Salt) | |
| 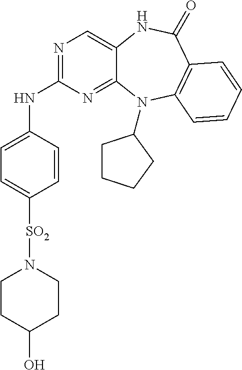 [SMILES: O=C1Nc2cnc(Nc3ccc(S(=O)(=O)N4CCC(O)CC4)cc3)nc2N(C2CCCC2)c2ccccc21] | IB-3 |
| (TFA Salt) | |
| 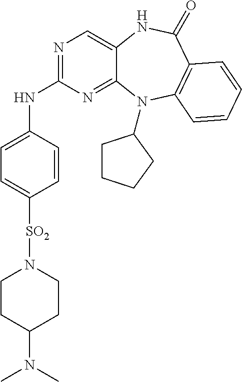 [SMILES: CN(C)C1CCN(S(=O)(=O)c2ccc(Nc3ncc4c(n3)N(C3CCCC3)c3ccccc3C(=O)N4)cc2)CC1] | IB-4 |
| 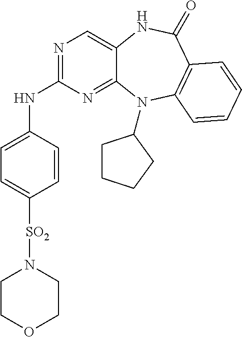 [SMILES: O=C1Nc2cnc(Nc3ccc(S(=O)(=O)N4CCOCC4)cc3)nc2N(C2CCCC2)c2ccccc21] | IB-5 |
| 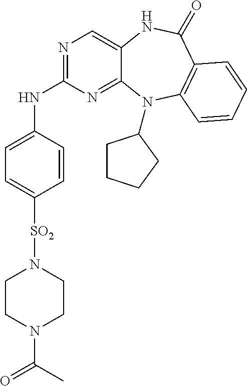 [SMILES: CC(=O)N1CCN(S(=O)(=O)c2ccc(Nc3ncc4c(n3)N(C3CCCC3)c3ccccc3C(=O)N4)cc2)CC1] | IB-6 |
| 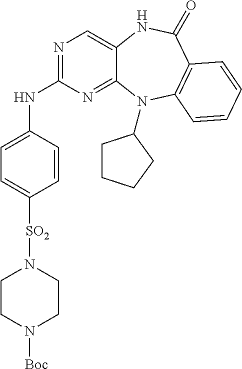 [SMILES: CC(C)(C)OC(=O)N1CCN(S(=O)(=O)c2ccc(Nc3ncc4c(n3)N(C3CCCC3)c3ccccc3C(=O)N4)cc2)CC1] | IB-7 |
| 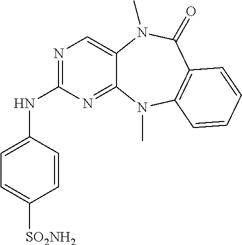 [SMILES: CN1C(=O)c2ccccc2N(C)c2nc(Nc3ccc(S(N)(=O)=O)cc3)ncc21] | IC-1 |
| 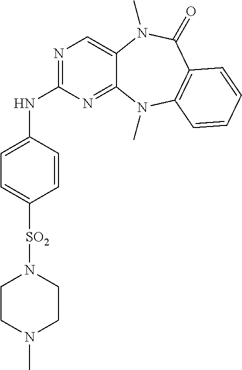 [SMILES: CN1CCN(S(=O)(=O)c2ccc(Nc3ncc4c(n3)N(C)c3ccccc3C(=O)N4C)cc2)CC1] | IC-2 |
| (TFA Salt) | |
| 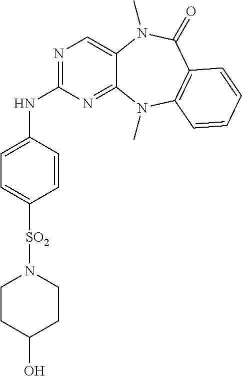 [SMILES: CN1C(=O)c2ccccc2N(C)c2nc(Nc3ccc(S(=O)(=O)N4CCC(O)CC4)cc3)ncc21] | IC-3 |
| (TFA Salt) | |
| 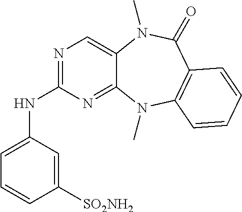 [SMILES: CN1C(=O)c2ccccc2N(C)c2nc(Nc3cccc(S(N)(=O)=O)c3)ncc21] | IC-4 |
| (TFA Salt) | |
| 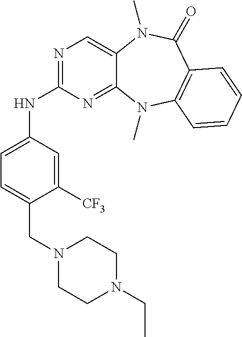 [SMILES: CCN1CCN(Cc2ccc(Nc3ncc4c(n3)N(C)c3ccccc3C(=O)N4C)cc2C(F)(F)F)CC1] | IC-5 |
| (TFA Salt) | |
| 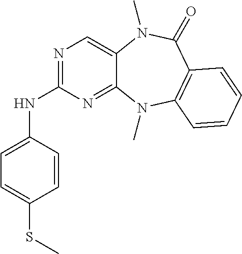 [SMILES: CSc1ccc(Nc2ncc3c(n2)N(C)c2ccccc2C(=O)N3C)cc1] | IC-6 |
| 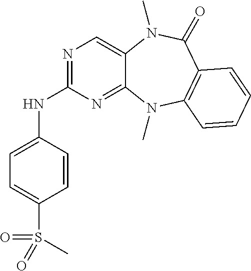 [SMILES: CN1C(=O)c2ccccc2N(C)c2nc(Nc3ccc(S(C)(=O)=O)cc3)ncc21] | IC-7 |
| 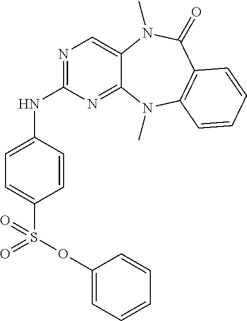 [SMILES: CN1C(=O)c2ccccc2N(C)c2nc(Nc3ccc(S(=O)(=O)Oc4ccccc4)cc3)ncc21] | IC-8 |
| 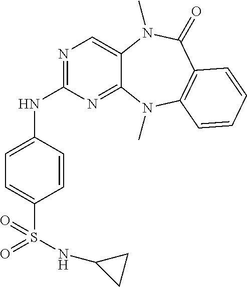 [SMILES: CN1C(=O)c2ccccc2N(C)c2nc(Nc3ccc(S(=O)(=O)NC4CC4)cc3)ncc21] | IC-9 |
| 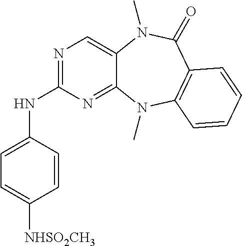 | IC-10 |
| (HCl Salt) | |
| 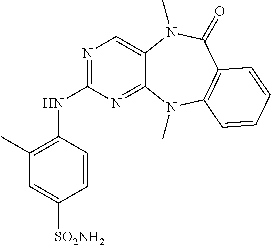 [SMILES: Cc1cc(S(N)(=O)=O)ccc1Nc1ncc2c(n1)N(C)c1ccccc1C(=O)N2C] | IC-11 |
| (TFA Salt) | |
| 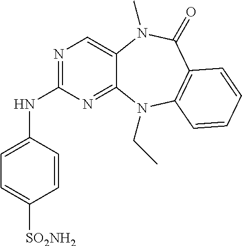 [SMILES: CCN1c2ccccc2C(=O)N(C)c2cnc(Nc3ccc(S(N)(=O)=O)cc3)nc21] | ID-1 |
| (HCl Salt) | |
| 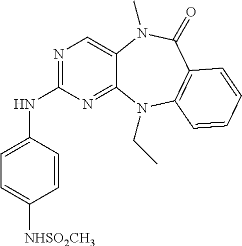 | ID-2 |
| (HCl Salt) | |
| 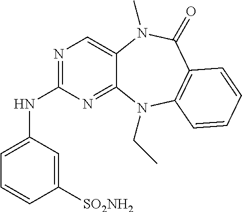 [SMILES: CCN1c2ccccc2C(=O)N(C)c2cnc(Nc3cccc(S(N)(=O)=O)c3)nc21] | ID-3 |
| (HCl Salt) | |
| 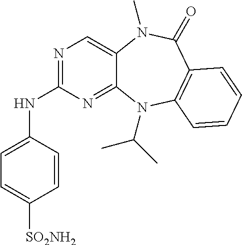 [SMILES: CC(C)N1c2ccccc2C(=O)N(C)c2cnc(Nc3ccc(S(N)(=O)=O)cc3)nc21] | IE-1 |
| (HCl Salt) | |
| 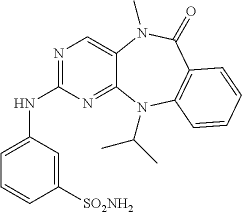 [SMILES: CC(C)N1c2ccccc2C(=O)N(C)c2cnc(Nc3cccc(S(N)(=O)=O)c3)nc21] | IE-2 |
| (TFA Salt) | |
| 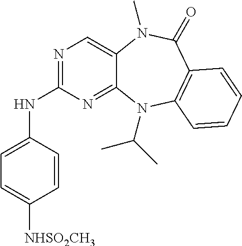 | IE-3 |
| (TFA Salt) | |
| 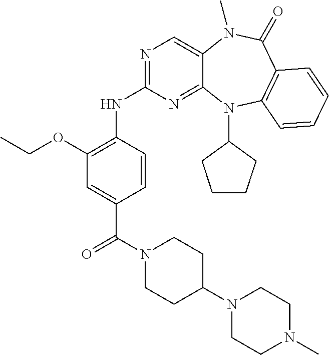 [SMILES: CCOc1cc(C(=O)N2CCC(N3CCN(C)CC3)CC2)ccc1Nc1ncc2c(n1)N(C1CCCC1)c1ccccc1C(=O)N2C] | IF-1 |
| 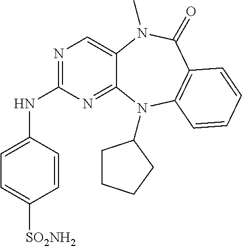 [SMILES: CN1C(=O)c2ccccc2N(C2CCCC2)c2nc(Nc3ccc(S(N)(=O)=O)cc3)ncc21] | IF-2 |
| 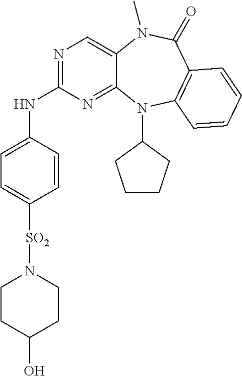 [SMILES: CN1C(=O)c2ccccc2N(C2CCCC2)c2nc(Nc3ccc(S(=O)(=O)N4CCC(O)CC4)cc3)ncc21] | IF-3 |
| 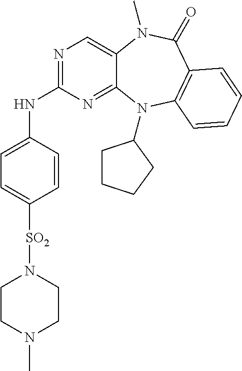 [SMILES: CN1CCN(S(=O)(=O)c2ccc(Nc3ncc4c(n3)N(C3CCCC3)c3ccccc3C(=O)N4C)cc2)CC1] | IF-4 |
| (TFA Salt) | |
| 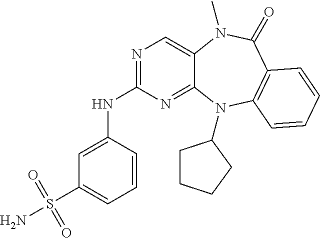 [SMILES: CN1C(=O)c2ccccc2N(C2CCCC2)c2nc(Nc3cccc(S(N)(=O)=O)c3)ncc21] | IF-5 |
| (TFA Salt) | |
| 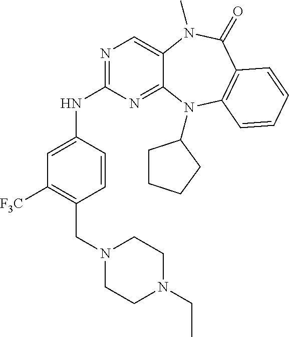 [SMILES: CCN1CCN(Cc2ccc(Nc3ncc4c(n3)N(C3CCCC3)c3ccccc3C(=O)N4C)cc2C(F)(F)F)CC1] | IF-6 |
| 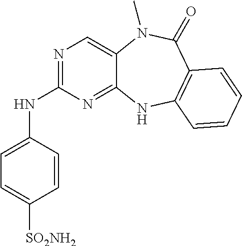 [SMILES: CN1C(=O)c2ccccc2Nc2nc(Nc3ccc(S(N)(=O)=O)cc3)ncc21] | IG-1 |
| (HCl Salt) | |
| 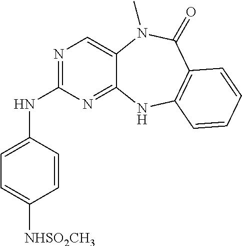 | IG-2 |
| (HCl Salt) | |
| 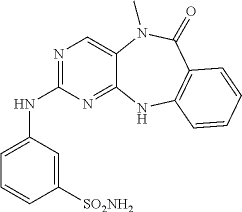 [SMILES: CN1C(=O)c2ccccc2Nc2nc(Nc3cccc(S(N)(=O)=O)c3)ncc21] | IG-3 |
| 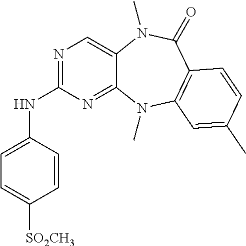 [SMILES: Cc1ccc2c(c1)N(C)c1nc(Nc3ccc(S(C)(=O)=O)cc3)ncc1N(C)C2=O] | IH-1 |
| 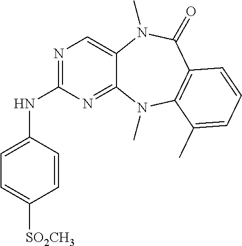 [SMILES: Cc1cccc2c1N(C)c1nc(Nc3ccc(S(C)(=O)=O)cc3)ncc1N(C)C2=O] | IH-2 |
| (TFA Salt) | |
| 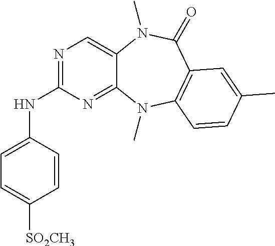 [SMILES: Cc1ccc2c(c1)C(=O)N(C)c1cnc(Nc3ccc(S(C)(=O)=O)cc3)nc1N2C] | IH-3 |
| (TFA Salt) | |
| 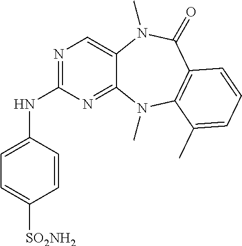 [SMILES: Cc1cccc2c1N(C)c1nc(Nc3ccc(S(N)(=O)=O)cc3)ncc1N(C)C2=O] | IH-4 |
| (TFA Salt) | |
| 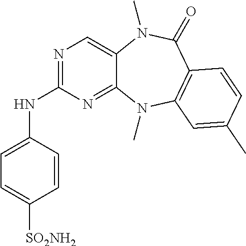 [SMILES: Cc1ccc2c(c1)N(C)c1nc(Nc3ccc(S(N)(=O)=O)cc3)ncc1N(C)C2=O] | IH-5 |
| (TFA Salt) | |
| 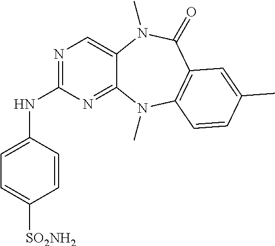 [SMILES: Cc1ccc2c(c1)C(=O)N(C)c1cnc(Nc3ccc(S(N)(=O)=O)cc3)nc1N2C] | IH-6 |
| (HCl Salt) | |
| 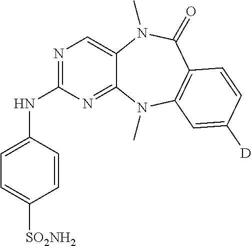 [SMILES: [2H]c1ccc2c(c1)N(C)c1nc(Nc3ccc(S(N)(=O)=O)cc3)ncc1N(C)C2=O] | IH-7 |
| (HCl Salt) | |
| 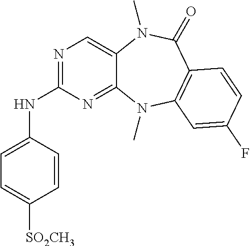 [SMILES: CN1C(=O)c2ccc(F)cc2N(C)c2nc(Nc3ccc(S(C)(=O)=O)cc3)ncc21] | IH-8 |
| 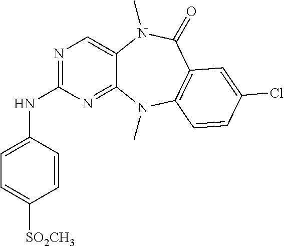 [SMILES: CN1C(=O)c2cc(Cl)ccc2N(C)c2nc(Nc3ccc(S(C)(=O)=O)cc3)ncc21] | IH-9 |
| 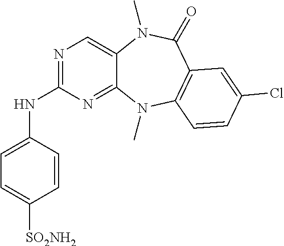 [SMILES: CN1C(=O)c2cc(Cl)ccc2N(C)c2nc(Nc3ccc(S(N)(=O)=O)cc3)ncc21] | IH-10 |
| (HCl Salt) | |
| 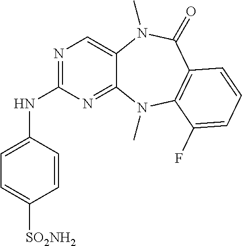 [SMILES: CN1C(=O)c2cccc(F)c2N(C)c2nc(Nc3ccc(S(N)(=O)=O)cc3)ncc21] | IH-11 |
| (HCl Salt) | |
| 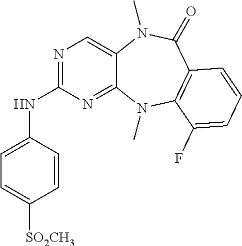 [SMILES: CN1C(=O)c2cccc(F)c2N(C)c2nc(Nc3ccc(S(C)(=O)=O)cc3)ncc21] | IH-12 |
| (HCl Salt) | |
| 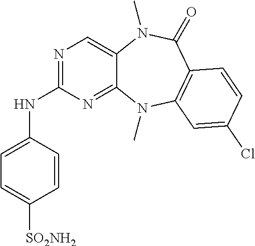 [SMILES: CN1C(=O)c2ccc(Cl)cc2N(C)c2nc(Nc3ccc(S(N)(=O)=O)cc3)ncc21] | IH-13 |
| (HCl Salt) | |
| 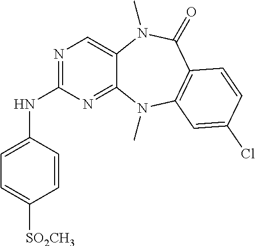 [SMILES: CN1C(=O)c2ccc(Cl)cc2N(C)c2nc(Nc3ccc(S(C)(=O)=O)cc3)ncc21] | IH-14 |
| (HCl Salt) | |

A fifth aspect, the inhibitors of MST1/2 protein kinases are represented by the following structural formula:

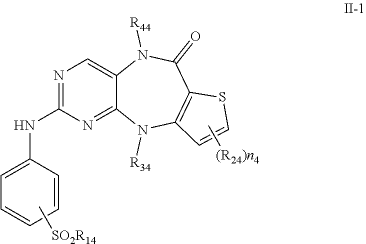

wherein n4 is selected from 0, 1 or 2;
R₁₄ is selected from:
1) selected from the group of five-membered heterocyclic or six-membered heterocyclic rings comprising one or more N, O and S heteroatoms, the five-membered heterocyclic or six-membered heterocyclic rings are optionally substituted with C1-C6 alkyl, C1-C6 alkoxy, hydroxy, amino, alkylamino, dialkylamino, C1-C6 acyl, cyano, optionally substituted C1-C6 alkyl, —O-C1-C6 alkyl, hydroxyl, hydroxyl C1-C6 alkyl, C1-C6 acyl, alkylamino, dialkylamino substituted heterocyclic group, including but not limited to: 4-N, N-dimethylamino-piperidinyl, 4-N, N-diethylamino-piperidinyl, 4-N, N-diisopropylamino-piperidinyl, 4-hydroxyl-piperidinyl, 4-(N-methylpiperazinyl) piperidinyl, 4-(N-ethyl-piperazinyl) piperidinyl, 4-(N-isopropyl-piperazinyl) piperidinyl, 4-(N-acetyl-piperazinyl) piperidinyl, 4-(N-tert-butoxyl formyl-piperazinyl) piperidinyl, 4-(N-methylsulfonyl-piperazinyl) piperidinyl, 4-(N-(2-hydroxylethyl) piperazinyl) piperidinyl, 4-(N-(2-cyanoethyl) piperazinyl) piperidinyl, 4-(N-(3-hydroxyl propyl) piperazinyl) piperidinyl, 4-(N-(2-N, N-dimethyl-3 ethyl) piperazinyl) piperidinyl, 4-(N-(2-N, N-diethyl-ethyl) piperazinyl) piperidinyl, 4-(N-(3-N, N-dimethyl-propyl) piperazinyl) piperidinyl, 4-(N-(3-N, N-diethyl-propyl) piperazinyl) piperidinyl, 4-(tertahydropyrrolyl) piperidinyl, 4-(3-N, N-dimethyl-tetrahydropyrrolyl) piperidinyl;
2) amino, cyclopropylamino, cyclobutylamino, cyclopentylamino, cyclohexylamino, N, N-dimethylamino, N, N-diethylamino, N, N-diisopropylamino, 2-N, N-dimethylamino ethylamino, 2-hydroxylethylamino, 2-morpholinyl-ethylamino, 2-(4-N-methylpiperazinyl) ethylamino, 3-N, N-dimethyl-aminopropyl amino, 3-N, N-diethylamino propylamino, 3-N, N-diisopropylamino-propylamino, 3-hydroxypropyl, 3-morpholinyl-propylamino, 3-(4-N-methylpiperazinyl) propylamino, N-methyl-piperidinyl-4-amino, N-ethylpiperidinyl-4-amino, N-isopropyl piperidinyl-4-amino, N-acetylpiperidinyl-4-amino; N-methyl-piperazinyl, N-ethyl-piperazinyl, N-isopropyl-piperazinyl, N-acetyl piperazinyl, N-tert-butoxyl formyl-piperazinyl, N-methylsulfonyl-piperazinyl, N-(2-hydroxylethyl) piperazinyl, N-(2-cyanoethyl) piperazinyl, N-(3-hydroxylpropyl) piperazinyl, N-(2-N, N-dimethylethyl) piperazinyl, N-(2-N, N-diethyl aminoethyl) piperazinyl, N-(3-N, N-dimethyl-propyl) piperazinyl, N-(3-N, N-diethyl-propyl) piperazinyl, 2-oxo-piperazin-4-yl, N—(N-methyl-4-piperidinyl) piperazinyl, N—(N-ethyl-4-piperidinyl) piperazinyl, N—(N-acetyl-4-piperidinyl) piperazinyl; morpholinyl, 3,5-dimethyl morpholinyl, thiomorpholinyl, tetrahydropyrrolyl, 3-N, N-dimethyl-tetrahydropyrrolyl, 3-N, N-diethyl-tetrahydropyrrolyl;
3) C1-C6 alkyl, optionally substituted by halogen, nitro, cyano;
4) C3-C7 cycloalkyl, which is optionally substituted by halogen, nitro, cyano;
   5) —O—C1-C6 alkyl, optionally substituted by halogen, nitro, amino, cyano;
6) —O—C3-C7 cycloalkyl, which is optionally substituted by halogen, nitro, amino, cyano; 7) C6-C10 aryl, which is optionally substituted by halogen, nitro, amino, cyano; —O-C6-C10 aryl, which is optionally substituted by halogen, nitro, amino, cyano;
8) C2-C6 alkenyl;
9) hydroxyl, 2-N, N-dimethylaminoethoxyl, 2-N, N-diethyl-aminoethoxyl, 2-N, N-diisopropyl-aminoethoxyl, 2-(N-methylpiperazinyl) ethoxyl, 2-(N-acetyl-piperazinyl) ethoxyl, 2-morpholinyl-ethoxyl, 2-thiomorpholinyl ethoxyl, 2-piperidinyl-ethoxyl, 3-N, N-dimethylamino-propoxyl, 3-N, N-diethylamino-propoxyl, 3-N, N-diisopropylamino propoxyl, 3-(N-methylpiperazinyl) propoxyl, 3-(N-acetyl-piperazinyl) propoxyl, 3-morpholinyl-propoxyl, 3-thiomorpholinyl propoxyl, 3-piperidinyl-propoxyl, 2-pyridyl-methoxyl, 3-pyridyl-methoxyl, 4-pyridyl methoxyl, phenylmethoxyl, monhalogen-substituted phenylmethoxyl, homodihalogen substituted phenylmethoxyl, heterodihalo-substituted phenylmethoxyl;
   R₂₄ is selected from:
   1) a hydrogen, halo, nitro, amino, cyano;
   2) C1-C6 alkyl, optionally substituted by halogen, nitro, amino, cyano; —O-C1-C6 alkyl, optionally substituted by halogen, nitro, amino, cyano; C1-C6 oxygen-containing alkyl;
   3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butylamido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl; R₃₄ is selected from:
   hydrogen; C1-C6 alkyl, optionally substituted by halogen, nitro, amino, cyano; C3-C7 cycloalkyl, which is optionally substituted by halogen, nitro, amino, cyano; R₄ is selected from:
   hydrogen; C1-C6 alkyl, optionally substituted by halogen, nitro, amino, cyano; C3-C7 cycloalkyl, which is optionally substituted by halogen, nitro, amino, cyano;
   Or a stereoisomer of the above compounds, a prodrug thereof, a pharmaceutically acceptable salt thereof, or a pharmaceutically acceptable solvate thereof.

A sixth aspect, the inhibitors of MST1/2 protein kinases are represented by the following structural formula:

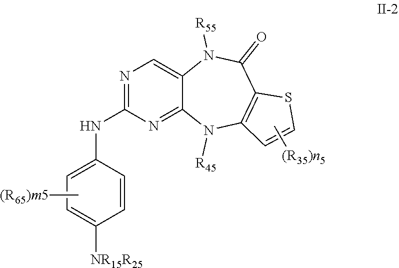

wherein m5 is selected from 0, 1, 2, 3 or 4;
n5 is selected from 0, 1 or 2;
R₁₅, R₂₅ are independently selected from:
1) hydrogen; C1-C6 alkyl, optionally substituted by halogen, nitro, amino, cyano; C3-C7 cycloalkyl, which is optionally substituted by halogen, nitro, amino, cyano; a C1-C6 oxygen-containing alkyl;
2) —SO₂C1-C6 alkyl, optionally substituted by halogen, nitro, amino, cyano; —SO₂C2-C6 alkenyl, which is optionally substituted with halogen, nitro, amino, cyano substituted; —COC1-C6 alkyl, optionally substituted by halogen, nitro, amino, cyano; —COC2-C6 alkenyl, which is optionally substituted by halogen, nitro, amino, cyano;
Or, together with R₁₅ and R₂₅ and the N atom to which they are attached forming a hexaheterocyclic ring that contains one or more heteroatoms selected from N, O and S, said hexahetrerocyclic ring is optionally substituted with C1-C6 alkyl, hydroxyl, or amino group;
R₃₅ is selected from:
1) a hydrogen, halo, nitro, amino, cyano;
2) C1-C6 alkyl, optionally substituted by halogen, nitro, amino, cyano; —O—C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, nbutoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
R₄₅ is selected from:

hydrogen; C1-C6 alkyl, optionally substituted by halogen, nitro, amino, cyano; C3-C7 cycloalkyl, which is optionally substituted by halogen, nitro, amino, cyano;

R₅₅ is selected from:
hydrogen; C1-C6 alkyl, optionally substituted by halogen, nitro, amino, cyano; C3-C7 cycloalkyl, which is optionally substituted by halogen, nitro, amino, cyano;
R₆₅ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methaylsulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl foramido, cyclopentyl foramido, methylsulfonamido, ethylsulfonamido, propylsulfonamido, isopropylsulfonamido, dimethyl phosphinyl, diethyl phosphinyl, diisopropyl phosphinyl;
Or a stereoisomer of the above compounds, a prodrug thereof, a pharmaceutically acceptable salt thereof, or a pharmaceutically acceptable solvate thereof.

A seventh aspect, the inhibitors of MST1/2 protein kinases are represented by the following structural formula:

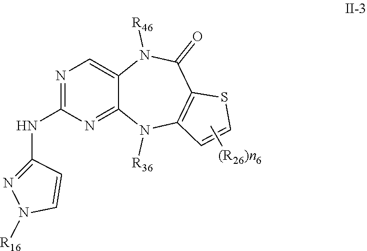

n6 is selected from 0, 1 or 2;
R₁₆ is selected from:
1) hydrogen; C1-C6 alkyl, optionally substituted by halogen, nitro, amino, cyano; C3-C7 cycloalkyl, which is optionally substituted by halogen, nitro, amino, cyano; a C1-C6 oxygen-containing alkyl;
2) —SO₂C1-C6 alkyl, optionally substituted by halogen, nitro, amino, cyano; —SO₂C2-C6 alkenyl, which is optionally substituted with halogen, nitro, amino, cyano substituted; —COC1-C6 alkyl, optionally substituted by halogen, nitro, amino, cyano; —COC2-C6 alkenyl, which is optionally substituted by halogen, nitro, amino, cyano;
3) piperidinyl, 4-N, N-dimethylamino-piperidinyl, 4-N, N-diethylamino-piperidinyl, 4-N, N-diisopropylamino-piperidinyl, 4-hydroxyl piperidinyl, 4-(N-methylpiperazinyl) piperidinyl, 4-(N-ethyl-piperazinyl) piperidinyl, 4-(N-isopropyl-piperazinyl) piperidine, 4-(N-acetyl-piperazinyl) piperidinyl, 4-(N-tert-butoxyl formyl-piperazinyl) piperidinyl, 4-(N-methylsulfonyl-piperazinyl) piperidinyl, 4-(N-(2-hydroxylethyl) piperazinyl) piperidinyl, 4-(N-(2-cyanoethyl) piperazinyl) piperidinyl, 4-(N-(3-hydroxylpropyl) piperazinyl) piperidinyl, 4-(N-(2-N, N-dimethyl-ethyl) piperazinyl) piperidinyl, 4-(N-(2-N, N-diethyl ethyl) piperazinyl) piperidinyl, 4-(N-(3-N, N-dimethyl-propyl) piperazinyl) piperidinyl, 4-(N-(3-N, N-diethyl-propyl) piperazinyl) piperidinyl, 4-(tetrahydropyrrolyl) piperidinyl, 4-(3-N, N-dimethyl-tetrahydropyrrolyl) piperidinyl;
R₂₆ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylesulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
R₃₆ is selected from: hydrogen; C1-C6 alkyl, optionally substituted by halogen, nitro, amino, cyano; C3-C7 cycloalkyl, which is optionally substituted with halogen, nitro, amino, cyano;
R₄₆ is selected from: hydrogen; C1-C6 alkyl, optionally substituted by halogen, nitro, amino, cyano; C3-C7 cycloalkyl, which is optionally substituted with halogen, nitro, amino, cyano;
Or a stereoisomer of the above compounds, a prodrug thereof, a pharmaceutically acceptable salt thereof, or a pharmaceutically acceptable solvate thereof.

An eighth aspect, the inhibitors of MST1/2 protein kinases are represented by the following structural formula:

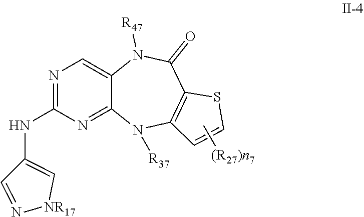

wherein n7 is selected from 0, 1 or 2;
R₁₇ is selected from:
1) hydrogen; C1-C6 alkyl, optionally substituted by halogen, nitro, amino, cyano; C3-C7 cycloalkyl, which is optionally substituted by halogen, nitro, amino, cyano; a C1-C6 oxygen-containing alkyl;
2) —SO₂C1-C6 alkyl, optionally substituted by halogen, nitro, amino, cyano; —SO₂C2-C6 alkenyl, which is optionally substituted with halogen, nitro, amino, cyano substituted; —COC1-C6 alkyl, optionally substituted by halogen, nitro, amino, cyano; —COC2-C6 alkenyl, which is optionally substituted by halogen, nitro, amino, cyano;
3) piperidinyl, 4-N, N-dimethylamino-piperidinyl, 4-N, N-diethylamino-piperidinyl, 4-N, N-diisopropylamino-piperidinyl, 4-hydroxyl piperidinyl, 4-(N-methylpiperazinyl) piperidinyl, 4-(N-ethyl-piperazinyl) piperidinyl, 4-(N-isopropyl-piperazinyl) piperidinyl, 4-(N-acetyl-piperazinyl) piperidinyl, 4-(N-tert-butoxyl formyl-piperazinyl) piperidinyl, 4-(N-methylsulfonyl-piperazinyl) piperidinyl, 4-(N-(2-hydroxylethyl) piperazinyl) piperidinyl, 4-(N-(2-cyanoethyl) piperazinyl) piperidinyl, 4-(N-(3-hydroxylpropyl) piperazinyl) piperidinyl, 4-(N-(2-N, N-dimethyl-ethyl) piperazinyl) piperidinyl, 4-(N-(2-N, N-diethyl ethyl) piperazinyl) piperidinyl, 4-(N-(3-N, N-dimethyl-propyl) piperazinyl) piperidinyl, 4-(N-(3-N, N-diethyl-propyl) piperazinyl) piperidinyl, 4-(tetrahydropyrrolyl) piperidinyl, 4-(3-N, N-dimethyl-tetrahydropyrrolyl) piperidinyl;
R₂₇ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
R₃₇ is selected from:
Hydrogen; C1-C6 alkyl, optionally substituted by halogen, nitro, amino, cyano; C3-C7 cycloalkyl, which is optionally substituted by halogen, nitro, amino, cyano;
R₄₇ is selected from:
Hydrogen; C1-C6 alkyl, optionally substituted by halogen, nitro, amino, cyano; C3-C7 cycloalkyl, which is optionally substituted by halogen, nitro, amino, cyano;
Or a stereoisomer of the above compounds, a prodrug thereof, a pharmaceutically acceptable salt thereof, or a pharmaceutically acceptable solvate thereof.

The inhibotors of MST1/2 protein kinases having the formula II are exemplified by the compounds in Table 2.

TABLE 2
MST1/2 protein kinase inhibitors having Formula II

| | |
|---|---|
| 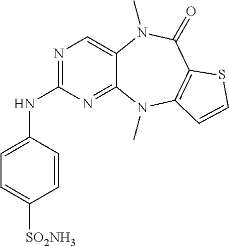 | II-1 |
| (TFA Salt) | |
| 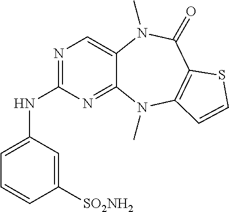 [SMILES: CN1C(=O)c2sccc2N(C)c2nc(Nc3cccc(S(N)(=O)=O)c3)ncc21] | II-2 |
| 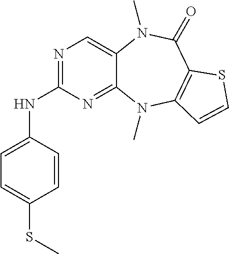 [SMILES: CSc1ccc(Nc2ncc3c(n2)N(C)c2ccsc2C(=O)N3C)cc1] | II-3 |
| 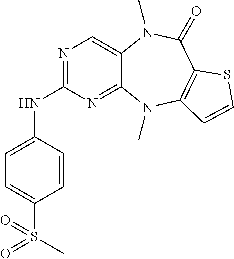 [SMILES: CN1C(=O)c2sccc2N(C)c2nc(Nc3ccc(S(C)(=O)=O)cc3)ncc21] | II-4 |
| 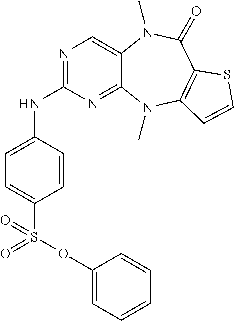 [SMILES: CN1C(=O)c2sccc2N(C)c2nc(Nc3ccc(S(=O)(=O)Oc4ccccc4)cc3)ncc21] | II-5 |
| 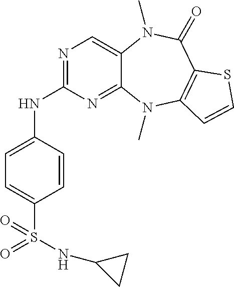 [SMILES: CN1C(=O)c2sccc2N(C)c2nc(Nc3ccc(S(=O)(=O)NC4CC4)cc3)ncc21] | II-6 |
| 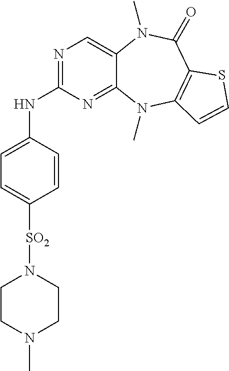 [SMILES: CN1CCN(S(=O)(=O)c2ccc(Nc3ncc4c(n3)N(C)c3ccsc3C(=O)N4C)cc2)CC1] | II-7 |
| 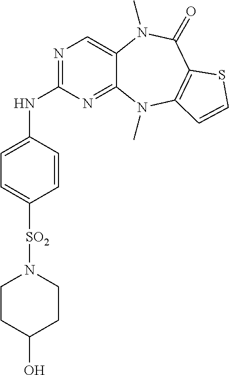 [SMILES: CN1C(=O)c2sccc2N(C)c2nc(Nc3ccc(S(=O)(=O)N4CCC(O)CC4)cc3)ncc21] | II-8 |
| (TFA Salt) | |
| 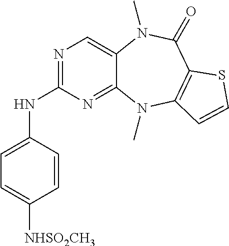 | II-9 |
| 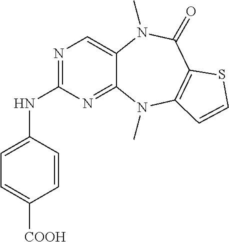 [SMILES: CN1C(=O)c2sccc2N(C)c2nc(Nc3ccc(C(=O)O)cc3)ncc21] | II-10 |
| (HCl Salt) | |
| 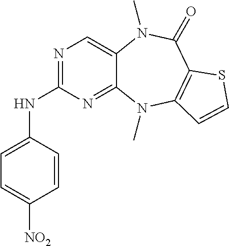 [SMILES: CN1C(=O)c2sccc2N(C)c2nc(Nc3ccc([N+](=O)[O-])cc3)ncc21] | II-11 |
| (HCl Salt) | |
| 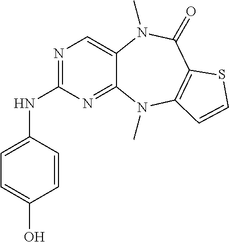 [SMILES: CN1C(=O)c2sccc2N(C)c2nc(Nc3ccc(O)cc3)ncc21] | II-12 |
| (HCl Salt) | |
| 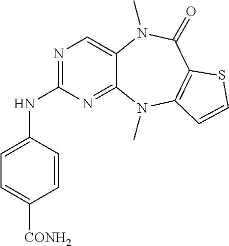 [SMILES: CN1C(=O)c2sccc2N(C)c2nc(Nc3ccc(C(N)=O)cc3)ncc21] | II-13 |
| (HCl Salt) | |
| 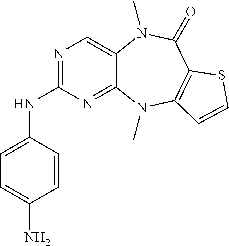 [SMILES: CN1C(=O)c2sccc2N(C)c2nc(Nc3ccc(N)cc3)ncc21] | II-14 |
| (TFA Salt) | |
| 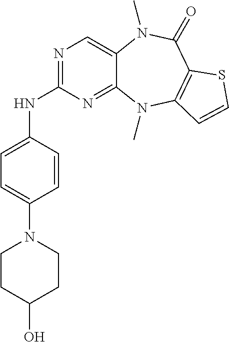 [SMILES: CN1C(=O)c2sccc2N(C)c2nc(Nc3ccc(N4CCC(O)CC4)cc3)ncc21] | II-15 |
| 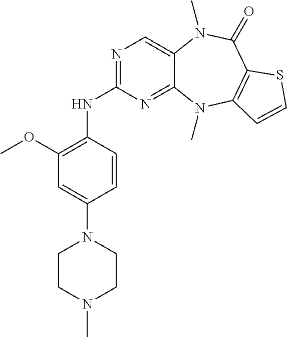 [SMILES: COc1cc(N2CCN(C)CC2)ccc1Nc1ncc2c(n1)N(C)c1ccsc1C(=O)N2C] | II-16 |
| 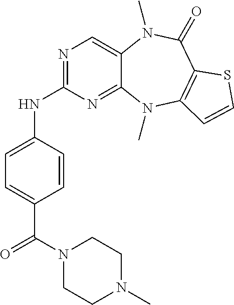 [SMILES: CN1CCN(C(=O)c2ccc(Nc3ncc4c(n3)N(C)c3ccsc3C(=O)N4C)cc2)CC1] | II-17 |
| (TFA Salt) | |
| 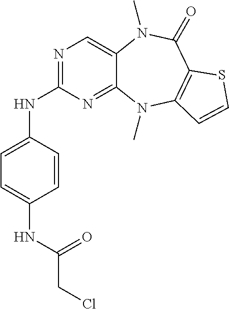 [SMILES: CN1C(=O)c2sccc2N(C)c2nc(Nc3ccc(NC(=O)CCl)cc3)ncc21] | II-18 |
| (TFA Salt) | |
| 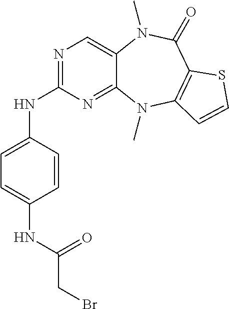 [SMILES: CN1C(=O)c2sccc2N(C)c2nc(Nc3ccc(NC(=O)CBr)cc3)ncc21] | II-19 |
| (TFA Salt) | |
| 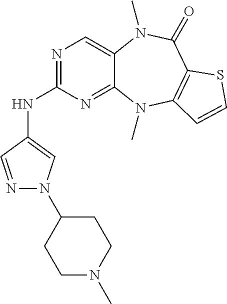 [SMILES: CN1CCC(n2cc(Nc3ncc4c(n3)N(C)c3ccsc3C(=O)N4C)cn2)CC1] | II-20 |
| 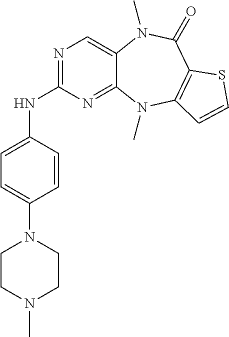 [SMILES: CN1CCN(c2ccc(Nc3ncc4c(n3)N(C)c3ccsc3C(=O)N4C)cc2)CC1] | II-21 |
| 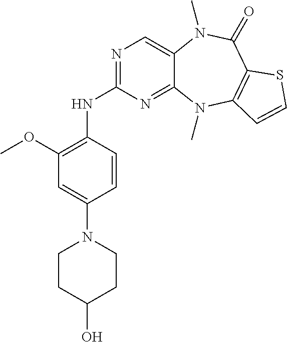 [SMILES: COc1cc(N2CCC(O)CC2)ccc1Nc1ncc2c(n1)N(C)c1ccsc1C(=O)N2C] | II-22 |
| 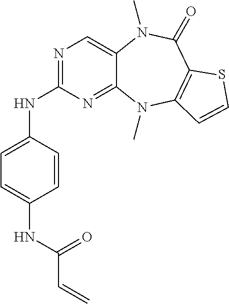 [SMILES: C=CC(=O)Nc1ccc(Nc2ncc3c(n2)N(C)c2ccsc2C(=O)N3C)cc1] | II-23 |
| (TFA Salt) | |
| 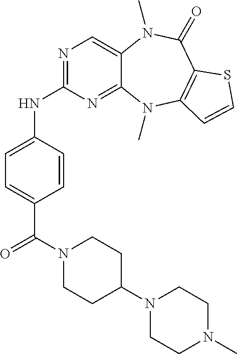 [SMILES: CN1CCN(C2CCN(C(=O)c3ccc(Nc4ncc5c(n4)N(C)c4ccsc4C(=O)N5C)cc3)CC2)CC1] | II-24 |
| 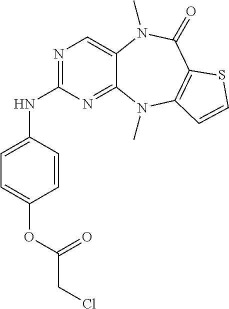 [SMILES: CN1C(=O)c2sccc2N(C)c2nc(Nc3ccc(OC(=O)CCl)cc3)ncc21] | II-25 |
| 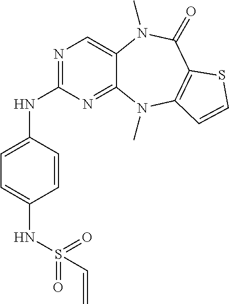 [SMILES: C=CS(=O)(=O)Nc1ccc(Nc2ncc3c(n2)N(C)c2ccsc2C(=O)N3C)cc1] | II-26 |
| (TFA Salt) | |
| 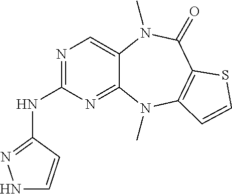 [SMILES: CN1C(=O)c2sccc2N(C)c2nc(Nc3cc[nH]n3)ncc21] | II-27 |
| 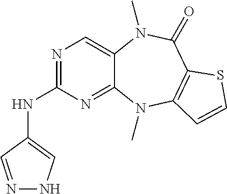 [SMILES: CN1C(=O)c2sccc2N(C)c2nc(Nc3cn[nH]c3)ncc21] | II-28 |
| 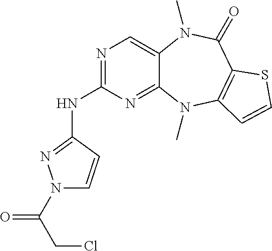 [SMILES: CN1C(=O)c2sccc2N(C)c2nc(Nc3ccn(C(=O)CCl)n3)ncc21] | II-29 |
| 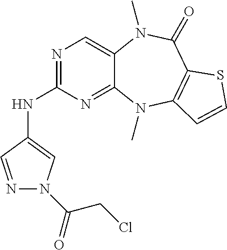 [SMILES: CN1C(=O)c2sccc2N(C)c2nc(Nc3cnn(C(=O)CCl)c3)ncc21] | II-30 |

A ninth aspect, the inhibitors of MST1/2 protein kinases are represented by the following structural formula:

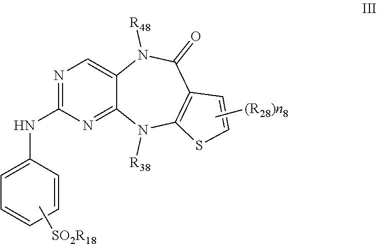

wherein n8 is selected from 0, 1 or 2;
R₁₈ is selected from:
1) selected from the group of five-membered heterocyclic or six-membered heterocyclic rings comprising one or more heteroatoms selected from N, O and S, the five-membered heterocyclic or six-membered heterocyclic rings are optionally substituted with C1-C6 alkyl, C1-C6 alkoxy, hydroxy, amino, C1-C6 acyl, a cyano, a substituted heterocyclic group, including but not limited to: 4-N, N-dimethylamino-piperidinyl, 4-N, N-diethylamino-piperidinyl, 4-N, N-diisopropylamino-piperidinyl, 4-hydroxyl-piperidinyl, 4-(N-methylpiperazinyl) piperidinyl, 4-(N-ethyl-piperazinyl) piperidinyl, 4-(N-isopropyl-piperazinyl) piperidinyl, 4-(N-acetyl-piperazinyl) piperidinyl, 4-(N-tert-butoxyl formyl-piperazinyl) piperidinyl, 4-(N-methylsulfonyl-piperazinyl) piperidinyl, 4-(N-(2-hydroxyethyl) piperazinyl) piperidinyl, 4-(N-(2-cyanoethyl) piperazinyl) piperidinyl, 4-(N-(3-hydroxyl propyl) piperazinyl) piperidinyl, 4-(N-(2-N, N-dimethyl-ethyl) piperazinyl) piperidinyl, 4-(N-(2-N, N-diethyl-ethyl) piperazinyl) piperidinyl, 4-(N-(3-N, N-dimethyl-propyl) piperazinyl) piperidinyl, 4-(N-(3-N, N-diethyl-propyl) piperazinyl) piperidinyl, 4-(tetrahydropyrrolyl) piperidinyl, 4-(3-N, N-dimethyl-tetrahydropyrrolyl) piperidinyl;
2) amino, cyclopropylamino, cyclobutylamino, cyclopentylamino, cyclohexylamino, N, N-dimethylamino, N, N-diethylamino, N, N-diisopropylamino, 2-N, N-dimethylethylamino, 2-hydroxyethylamino, 2-morpholinyl-ethylamino, 2-(4-N-methylpiperazinyl) ethylamino, 3-N, N-dimethyl-aminopropyl amino, 3-N, N-diethylamino propylamino, 3-N, N-diisopropylpropylamino, 3-hydroxypropyl amino, 3-morpholinyl-propylamino, 3-(4-N-methylpiperazinyl) propylamino, N-methylpiperidiny-4-amino, N-ethylpiperidinyl-4-amino, N-isopropyl piperidinyl-4-amino, N-acetylpiperidinyl-4-amino; N-methyl-piperazinyl, N-ethyl-piperazinyl, N-isopropyl-piperazinyl, N-acetyl piperazinyl, N-tert-butoxyl formyl-piperazinyl, N-methylsulfonyl-piperazinyl, N-(2-hydroxylethyl) piperazinyl, N-(2-cyanoethyl) piperazinyl, N-(3-hydroxylpropyl) piperazinyl, N-(2-N, N-dimethylethyl) piperazinyl, N-(2-N, N-diethyl aminoethyl) piperazinyl, N-(3-N, N-dimethyl-propyl) piperazinyl, N-(3-N, N-diethyl-propyl) piperazinyl, 2-oxo-piperazin-4-yl, N—(N-methyl-4-piperidinyl) piperazinyl, N—(N-ethyl-4-piperidinyl) piperazinyl, N—(N-acetyl-4-piperidinyl) piperazinyl; morpholinyl, 3,5-dimethyl morpholinyl, thiomorpholinyl, tetrahydropyrrolyl, 3-N, N-dimethyl-tetrahydropyrrolyl, 3-N, N-diethyl-tetrahydropyrrolyl;
3) C1-C6 alkyl, optionally substituted by halogen, nitro, cyano;
4) C3-C7 cycloalkyl, which is optionally substituted by halogen, nitro, cyano;
5) —O-C1-C6 alkyl, optionally substituted by halogen, nitro, amino, cyano;
6) —O-C3-C7 cycloalkyl, which is optionally substituted by halogen, nitro, amino, cyano;
7) C6-C10 aryl, which is optionally substituted by halogen, nitro, amino, cyano; —O-C6-C10 aryl, which is optionally substituted by halogen, nitro, amino, cyano;
8) C2-C6 alkenyl;
9) hydroxyl, 2-N, N-dimethylaminoethoxyl, 2-N, N-diethyl-aminoethoxyl, 2-N, N-diisopropyl-aminoethoxyl, 2-(N-methylpiperazine-yl) ethoxyl, 2-(N-acetyl-piperazinyl) ethoxyl, 2-morpholinyl-ethoxyl, 2-thiomorpholinyl ethoxyl, 2-piperidinyl-ethoxyl, 3-N, N-dimethylamino-propoxyl, 3-N, N-diethylamino-propoxyl, 3-N, N-diisopropylamino propoxyl, 3-(N-methylpiperazinyl) propoxyl, 3-(N-acetyl-piperazinyl) propoxyl, 3-morpholinyl-propoxyl, 3-thiomorpholinyl propoxyl, 3-piperidinyl-propoxyl, 2-pyridyl-methoxyl, 3-pyridyl-methoxyl, 4-pyridyl methoxyl, phenylmethoxyl, monohalogen-substituted phenylmethoxyl, homodihalogen-substituted phenylmethoxyl, heterodihalogen-substituted phenylmethoxyl;
R₂₈ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
R₃₈ is selected from: hydrogen; C1-C6 alkyl, optionally substituted by halogen, nitro, amino, cyano; C3-C7 cycloalkyl, which is optionally substituted with halogen, nitro, amino, cyano;
R₄₈ is selected from: hydrogen; C1-C6 alkyl, optionally substituted by halogen, nitro, amino, cyano; C3-C7 cycloalkyl, which is optionally substituted with halogen, nitro, amino, cyano;
Or a stereoisomer of the above compounds, a prodrug thereof, a pharmaceutically acceptable salt thereof, or a pharmaceutically acceptable solvate thereof.

Inhibitors of MST1/2 protein kinases having the formula III are exemplified by the compounds in Table 3.

TABLE 3
MST1/2 protein kinase inhibitors having Formula III

| | |
|---|---|
| 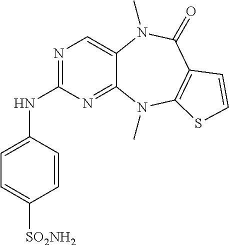 [SMILES: CN1C(=O)c2ccsc2N(C)c2nc(Nc3ccc(S(N)(=O)=O)cc3)ncc21] | III-1 |
| (TFA Salt) | |
| 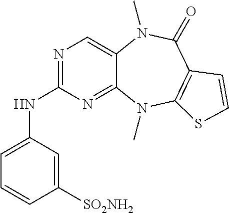 [SMILES: CN1C(=O)c2ccsc2N(C)c2nc(Nc3cccc(S(N)(=O)=O)c3)ncc21] | III-2 |
| 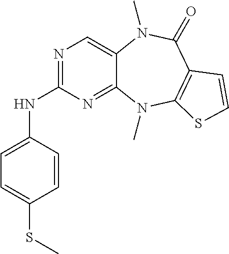 [SMILES: CSc1ccc(Nc2ncc3c(n2)N(C)c2sccc2C(=O)N3C)cc1] | III-3 |
| 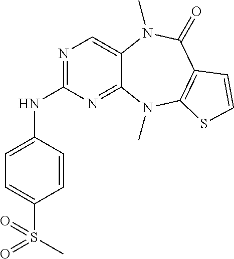 [SMILES: CN1C(=O)c2ccsc2N(C)c2nc(Nc3ccc(S(C)(=O)=O)cc3)ncc21] | III-4 |

A tenth aspect, the inhibitors of MST1/2 protein kinases are represented by the following structural formula:

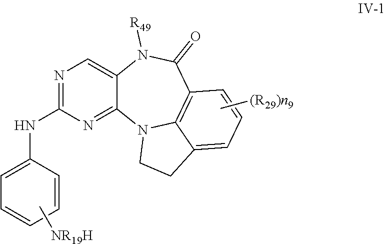

wherein n9 is selected from 0, 1, 2 or 3;
R₁₉ is selected from:
1) hydrogen; C1-C6 alkyl, optionally substituted by halogen, nitro, amino, cyano; C3-C7 cycloalkyl, which is optionally substituted by halogen, nitro, amino, cyano; a C1-C6 oxygen-containing alkyl;
2) —SO₂C1-C6 alkyl, optionally substituted by halogen, nitro, amino, cyano; —SO₂C2-C6 alkenyl, which is optionally substituted with halogen, nitro, amino, cyano substituted; —COC1-C6 alkyl, optionally substituted by halogen, nitro, amino, cyano; —COC2-C6 alkenyl, which is optionally substituted by halogen, nitro, amino, cyano;
R₂₉ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylaminoformyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
R₄₉ is selected from: hydrogen; C1-C6 alkyl, optionally substituted by halogen, nitro, amino, cyano; C3-C7 cycloalkyl, which is optionally substituted with halogen, nitro, amino, cyano;
Or a stereoisomer of the above compounds, a prodrug thereof, a pharmaceutically acceptable salt thereof, or a pharmaceutically acceptable solvate thereof.
In some embodiments, R₁₉ is selected from hydrogen; C1-C6 alkyl; C3-C7 cycloalkyl; —SO₂C1-C6 alkyl; —SO₂C2-C6 alkenyl group; —COC1-C6 alkyl, which is optionally substituted by halogen, nitro, amino, cyano; —COC2-C6 alkenyl, which is optionally substituted by halogen, nitro, amino, cyano.

An eleventh aspect, the inhibitors of MST1/2 protein kinases are represented by the following structural formula:

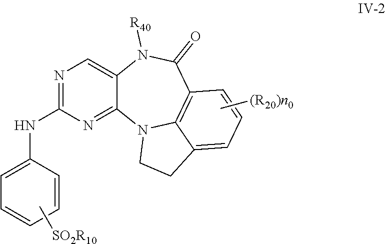

wherein n₀ is selected from 0, 1, 2 or 3;
R₁₀ is selected from:
1) selected from the group of five-membered heterocyclic or six-membered heterocyclic rings comprising one or more heteroatoms selected from N, O and S, the five-membered heterocyclic or six-membered heterocyclic rings are optionally substituted with C1-C6 alkyl, C1-C6 alkoxy, hydroxy, amino, alkylamino, dialkylamino, C1-C6 acyl, cyano, optionally substituted C1-C6 alkyl, —O-C1-C6 alkyl, hydroxy, hydroxy C1-C6 alkyl, C1-C6 acyl, alkylamino, dialkylamino substituted heterocyclic group, including but not limited to: 4-N, N-dimethylamino-piperidinyl, 4-N, N-diethylamino-piperidinyl, 4-N, N-diisopropylamino-piperidinyl, 4-hydroxyl-piperidinyl, 4-(N-methylpiperazinyl) piperidinyl, 4-(N-ethyl-piperazinyl) piperidinyl, 4-(N-isopropyl-piperazinyl) piperidinyl, 4-(N-acetyl-piperazinyl) piperidinyl, 4-(N-tert-butoxyl formyl-piperazinyl) piperidinyl, 4-(N-methylsulfonyl-piperazinyl) piperidinyl, 4-(N-(2-hydroxylethyl) piperazinyl) piperidinyl, 4-(N-(2-cyanoethyl) piperazinyl) piperidinyl, 4-(N-(3-hydroxyl propyl) piperazinyl) piperidinyl, 4-(N-(2-N, N-dimethyl-ethyl) piperazinyl) piperidinyl, 4-(N-(2-N, N-diethyl-ethyl) piperazinyl) piperidinyl, 4-(N-(3-N, N-dimethyl-propyl) piperazinyl) piperidinyl, 4-(N-(3-N, N-diethyl-propyl) piperazinyl) piperidinyl, 4-(tetrahydropyrrolyl) piperidinyl, 4-(3-N, N-dimethyl-tetrahydropyrrolyl) piperidinyl; 2) amino, cyclopropylamino, cyclobutylamino, cyclopentylamino, cyclohexylamino, N, N-dimethylamino, N, N-diethylamino, N, N-diisopropylamino, 2-N, N-dimethylaminoethyl, 2-hydroxylethylamino, 2-morpholinyl-ethylamino, 2-(4-N-methylpiperazinyl) ethylamino, 3-N, N-dimethylaminopropyl amino, 3-N, N-diethylaminopropyl amino, 3-N, N-diisopropylamino-propyl amino, 3-hydroxylpropyl amino, 3-morpholinyl-propylamino, 3-(4-N-methylpiperazinyl) propylamino, N-methyl-piperidinyl-4-amino, N-ethylpiperidinyl-4-amino, N-isopropyl piperidinyl-4-amino, N-acetylpiperidinyl-4-amino; N-methyl-piperazinyl, N-ethyl-piperazinyl, N-isopropyl-piperazinyl, N-acetyl piperazinyl, N-tert-butoxyl formyl-piperazinyl, N-methylsulfonyl-piperazinyl, N-(2-hydroxylethyl) piperazinyl, N-(2-cyanoethyl) piperazinyl, N-(3-hydroxylpropyl) piperazinyl, N-(2-N, N-dimethylethyl) piperazinyl, N-(2-N, N-diethyl aminoethyl) piperazinyl, N-(3-N, N-dimethyl-propyl) piperazinyl, N-(3-N, N-diethyl-propyl) piperazinyl, 2-oxo-piperazin-4-yl, N—(N-methyl-4-piperidinyl) piperazinyl, N—(N-ethyl-4-piperidinyl) piperazinyl, N—(N-acetyl-4-piperidinyl) piperazinyl; morpholinyl, 3,5-dimethyl morpholinyl, thiomorpholinyl, tetrahydropyrrolyl, 3-N, N-dimethyl-tetrahydropyrrolyl, 3-N, N-diethyl-tetrahydropyrrolyl;
3) C1-C6 alkyl, optionally substituted by halogen, nitro, cyano;
4) C3-C7 cycloalkyl, which is optionally substituted by halogen, nitro, cyano;
5) —O-C1-C6 alkyl, optionally substituted by halogen, nitro, amino, cyano;
6) —O-C3-C7 cycloalkyl, which is optionally substituted by halogen, nitro, amino, cyano;
7) C6-C10 aryl group, which is optionally substituted by halogen, nitro, amino, cyano; —O-C6-C10 aryl, which is optionally substituted by halogen, nitro, amino, cyano;
8) C2-C6 alkenyl group;
9) hydroxyl, 2-N, N-dimethylaminoethoxyl, 2-N, N-diethyl-aminoethoxyl, 2-N, N-diisopropyl-aminoethoxyl, 2-(N-methylpiperazinyl) ethoxyl, 2-(N-acetyl-piperazinyl) ethoxyl, 2-morpholinyl-ethoxyl, 2-thiomorpholinyl ethoxyl, 2-piperidinyl-ethoxyl, 3-N, N-dimethylamino-propoxyl, 3-N, N-diethylamino-propoxyl, 3-N, N-diisopropylamino propoxyl, 3-(N-methylpiperazinyl) propoxyl, 3-(N-acetyl-piperazinyl) propoxyl, 3-morpholinyl-propoxyl, 3-thiomorpholinyl propoxyl, 3-piperidinyl-propoxyl, 2-pyridyl methoxyl, 3-pyridyl-methoxyl, 4-pyridyl methoxyl, phenylmethoxyl, monohalogen-substituted phenylmethoxyl, homodihalogen-substituted phenyl methoxyl, heterodihalo-substituted phenylmethoxyl;
R₂₀ is selected from:
1) a hydrogen, halo, nitro, amino, cyano;
2) C1-C6 alkyl, optionally substituted by halogen, nitro, amino, cyano; —O-C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
R₄₀ is selected from: hydrogen; C1-C6 alkyl, optionally substituted by halogen, nitro, amino, cyano; C3-C7 cycloalkyl, which is optionally substituted with halogen, nitro, amino, cyano;
Or a stereoisomer of the above compounds, a prodrug thereof, a pharmaceutically acceptable salt thereof, or a pharmaceutically acceptable solvate thereof.

Inhibitors of MST1/2 protein kinases having the formula IV are exemplified by the compounds in Table 4.

TABLE 4
MST1/2 protein kinase inhibitors having Formula IV

| | |
|---|---|
| 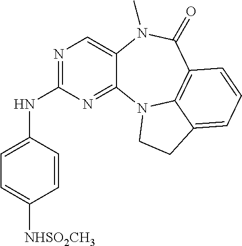 | IV-1 |
| (TFA Salt) | |
| 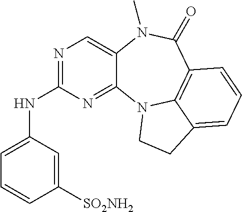 [SMILES: CN1C(=O)c2cccc3c2N(CC3)c2nc(Nc3cccc(S(N)(=O)=O)c3)ncc21] | IV-2 |
| (TFA Salt) | |
| 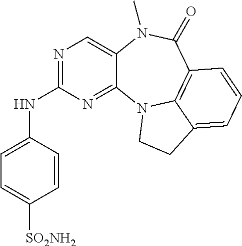 [SMILES: CN1C(=O)c2cccc3c2N(CC3)c2nc(Nc3ccc(S(N)(=O)=O)cc3)ncc21] | IV-3 |
| (TFA Salt) | |

Some of the inhibitors of the MST1/2 protein kinases have activities in inhibiting MST1 and MST2 as shown in Table 5.

TABLE 5
IC50 of the MST1/2 protein kinase inhibitors

| No | MST1 IC₅₀ (μM) | MST2 IC₅₀ (μM) |
|---|---|---|
| IA-2 | 2.38 | 14.80 |
| IA-4 | 2.51 | 2.74 |
| IA-5 | 24.43 | 23.30 |
| IA-6 | >30.00 | >30.00 |
| IB-1 | >30.00 | >30.00 |
| 1B-2 | >30.00 | >30.00 |
| 1B-3 | >30.00 | >30.00 |
| IC-1 | 0.07 | 0.11 |
| IC-2 | 2.32 | 7.80 |
| IC-3 | 0.67 | 0.52 |
| IC-4 | >30.00 | >30.00 |
| IC-5 | 1.91 | 1.80 |
| IC-7 | 0.50 | 0.09 |
| IC-8 | 13.50 | 0.20 |
| IC-9 | 0.18 | 2.33 |
| IC-10 | 0.24 | 0.08 |
| ID-1 | 0.04 | 0.11 |
| ID-2 | 0.08 | 0.39 |
| ID-3 | 8.65 | >30.00 |
| IE-1 | 0.83 | 2.45 |
| IE-2 | 15.76 | >30.00 |
| IE-3 | 2.72 | 8.21 |
| IF-2 | 0.63 | 3.86 |
| IF-3 | 2.44 | 15.69 |
| IF-4 | 6.46 | >30.00 |
| IF-5 | >30.00 | >30.00 |
| IF-6 | 9.76 | >30.00 |
| IG-1 | 1.55 | 2.70 |
| IG-2 | 5.80 | 14.64 |
| IG-3 | >30.00 | >30.00 |
| IH-1 | 5.01 | 4.81 |
| IH-2 | 0.89 | 1.68 |
| IH-4 | 0.15 | 0.19 |
| IH-5 | 2.40 | 2.73 |
| IH-6 | >30.00 | >30.00 |
| IH-3 | >30.00 | >30.00 |
| IH-7 | 3.12 | 2.49 |
| IH-8 | 2.17 | 3.36 |
| IH-9 | 5.67 | 11.63 |
| IH-10 | 5.93 | >30.00 |
| IH-11 | 0.23 | 0.44 |
| IH-12 | 1.66 | 1.94 |
| IH-13 | 7.82 | 9.47 |
| IH-14 | >30.00 | 22.53 |
| II-1 | 0.14 | 0.04 |
| II-2 | 2.71 | >30.00 |
| II-3 | 0.85 | 1.72 |
| II-4 | 0.17 | 0.22 |
| II-5 | 4.64 | 8.89 |
| II-6 | 0.26 | 0.02 |
| II-7 | 1.10 | 0.15 |
| II-8 | 0.08 | 1.03 |
| II-10 | 1.02 | 1.97 |
| II-11 | >30.00 | >30.00 |
| II-12 | 0.57 | 0.22 |
| II-13 | 0.33 | 0.26 |
| II-14 | 2.16 | 2.75 |
| II-15 | 0.65 | 1.99 |
| II-16 | >30.00 | >30.00 |
| II-17 | 0.61 | 2.51 |
| II-18 | 3.34 | 13.00 |
| II-19 | 3.03 | 12.59 |
| II-20 | 0.11 | 2.32 |
| II-21 | 0.25 | 3.10 |
| II-22 | >30.00 | >30.00 |
| II-23 | 0.77 | 4.76 |
| II-24 | 0.22 | 2.25 |
| II-25 | 0.58 | 2.24 |
| II-27 | 3.44 | >30.00 |
| II-26 | 0.82 | 0.41 |
| II-28 | 0.54 | 0.36 |
| II-30 | 0.81 | 0.44 |
| IV-1 | 6.07 | 3.97 |
| IV-2 | >30.00 | >30.00 |
| IV-3 | 0.43 | 1.61 |

The inhibitors of MST1/2 protein kinases also encompass the pharmaceutically acceptable salts of the compounds described above, including inorganic or organic acid salts, wherein the inorganic salt is a hydrochloride, hydrobromide, hydroiodide, nitrate, bicarbonate, and salts of carbonates, sulfates or phosphates, the organic acid salt is a formate, acetate, propionate, benzoate, maleate, fumarate, succinate, tartrate, citrate, ascorbate, alpha-ketoglutarate, alpha-glycerophosphate, alkyl sulfonate or aryl sulfonate. Preferably, the alkyl sulfonate is methylsulfonate or ethylsulfonate; and aryl sulfonate is benzylsulfonate or p-toluenylsulfonate.

In another aspect, the inhibitors of Mst1/2 protein kinases also encompass prodrugs of the compounds described above.

The inhibitors of Mst1/2 protein kinases may be formulated into a pharmaceutical composition, which also comprises one or more suitable pharmaceutical excipients, such as, carriers, diluents, fillers, buffers, bulking agents, stabilizers, solubilizers, and the like. The pharmaceutical composition may be in hard or soft shell capsules, swallowable tablets, buccal tablets, pills, troches, elixirs, suspensions, syrups, wafers, and the like formulations. For instance, tablets and pills may be coated with gelatin, wax, shellac or sugar. In addition, the pharmaceutical composition may be in an injection or infusion formulation of solution or dispersion suitable for sterile injectable or infusible formulation of the instant (optionally encapsulated in liposomes) in sterile aqueous solutions or dispersions or sterile powders.

The pharmaceutical composition may be in unit dosage form, which unit dosage form is a physically discrete unit containing a unit dose, suitable for administration to humans and other mammals. The unit dosage form can be a capsule or tablet, or a lot of capsules or tablets. The pharmaceutical composition may also have new dosage forms such as liposomes, microspheres and nanospheres, such as using fine particle dispersion comprising polymeric micelles (polymeric micelles), nanoemulsion (nanoemulsion), submicron emulsion (submicroemuls micro capsule (microcapsule), microspheres (microsphere), liposomes (liposomes) and lipid vesicles (niosomes) (also known as non-ionic surfactant vesicles) in the manufacture of a medicament and the like.

More examples of dosage forms of the pharmaceutical compositions include tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient. See Pharmaceutical Dosage Forms: Parenteral Medications, vol. 1, 2nd ed., Avis et al., Eds., Mercel Dekker, New York, N. Y. 1992.

In some embodiments, poorly soluble inhibitors of MST1/2 protein kinases may be incorporated into liquid dosage forms (and dosage forms suitable for reconstitution) with the aid of solubilizing agents, emulsifiers and surfactants such as, but not limited to, cyclodextrins (e.g., α-cyclodextrin, β-cyclodextrin, Captisol®, and Encapsin® (see, e.g., Davis and Brewster, Nat. Rev. Drug Disc. 3:1023-1034 (2004)), Labrasol®, Labrafil®, Labrafac®, cremafor, and non-aqueous solvents, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, dimethyl sulfoxide (DMSO), biocompatible oils (e.g., cottonseed, groundnut, corn, germ, olive, castor, and sesame oils), glycerol, tetrahydrofurfuryl alcohol, polyethylene glycols, fatty acid esters of sorbitan, and mixtures thereof (e.g., DMSO:cornoil).

The pharmaceutical composition may be in parenteral dosage forms that can be administered to patients by various routes including intramuscular, intravenous (including bolus injection), subcutaneous, intraperitoneal, subdermal, intradermal, intraarticular, intrathecal, sublingual, oral and the like. Examples of parenteral dosage forms include solutions ready for injection, dry products ready to be dissolved or suspended in a pharmaceutically acceptable vehicle for injection, suspensions ready for injection, and emulsions. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

The pharmaceutical composition may be formulated to enhance bioavailability of the inhibitors of MST1/2 protein kinases. An increase in bioavailability of the MST1/2 protein kinase inhibitors may be achieved by complexation of the inhibitors with one or more bioavailability or absorption enhancing agents or in bioavailability or absorption enhancing formulations. Such bioavailability or absorption enhancing agents include, but are not limited to, various surfactants such as various triglycerides, such as from butter oil, monoglycerides, such as of stearic acid and vegetable oils, esters thereof, esters of fatty acids, propylene glycol esters, the polysorbates, sodium lauryl sulfate, sorbitan esters, sodium sulfosuccinate, among other compounds. By altering the surfactant properties of the inhibitors, it is possible to, for example, allow the inhibitors to have greater intestinal contact over a longer period of time that increases uptake and reduces side effects.

Further examples of such bioavailability or absorption enhancing agents include carrier molecules such as cyclodextrin and derivatives thereof, well known in the art for their potential as complexation agents capable of altering the physicochemical attributes of the inhibitors. For example, cyclodextrins may stabilize (both thermally and oxidatively), reduce the volatility of, and alter the solubility of, the inhibitors with which they are complexed. Cyclodextrins are cyclic molecules composed of glucopyranose ring units that form toroidal structures. The interior of the cyclodextrin molecule is hydrophobic and the exterior is hydrophilic, making the cyclodextrin molecule water-soluble. The degree of solubility can be altered through substitution of the hydroxyl groups on the exterior of the cyclodextrin. Similarly, the hydrophobicity of the interior can be altered through substitution, though generally the hydrophobic nature of the interior allows accommodation of relatively hydrophobic guests within the cavity. Examples of cyclodextrin derivatives include sulfobutylcyclodextrin, maltosylcyclodextrin, hydroxypropylcyclodextrin, and salts thereof.

Accommodation of one molecule within another is known as complexation and the resulting product is referred to as an inclusion complex. Complexation of the inhibitors of MST1/2 protein kinases with a carrier molecule such as cyclodextrin to form an inclusion complex may thereby reduce the dose of the inhibitors of MST1/2 protein kinases needed for therapeutic efficacy by enhancing the bioavailability of the administered inhibitors of MST1/2 protein kinases.

The pharmaceutical composition may also be in a microemulsion to enhance bioavailability of the inhibitors of MST1/2 protein kinases. A microemulsion is a fluid and stable homogeneous solution composed of four major constituents, respectively, a hydrophilic phase, a lipophilic phase, at least one surfactant (SA) and at least one cosurfactant (CoSA). A surfactant is a chemical compound possessing two groups, the first is polar or ionic, which has a great affinity for water, the second contains a longer or shorter aliphatic chain and is hydrophobic. These chemical compounds having marked hydrophilic character are intended to cause the formation of micelles in aqueous or oily solution. Examples of suitable surfactants include mono-, di- and triglycerides and polyethylene glycol (PEG) mono- and diesters. A cosurfactant, also sometimes known as “co-surface-active agent”, is a chemical compound having hydrophobic character, intended to cause the mutual solubilization of the aqueous and oily phases in a microemulsion. Examples of suitable co-surfactants include ethyl diglycol, lauric esters of propylene glycol, oleic esters of polyglycerol, and related compounds.

The pharmaceutical composition may be formulated with various polymers to enhance bioavailability of the inhibitors of MST1/2 protein kinases by increasing adhesion to mucosal surfaces, by decreasing the rate of degradation by hydrolysis or enzymatic degradation of the inhibitors, and by increasing the surface area of the inhibitors relative to the size of the particle. Suitable polymers can be natural or synthetic, and can be biodegradable or non-biodegradable. Representative natural polymers include proteins such as zein, modified zein, casein, gelatin, gluten, serum albumin, and collagen, polysaccharides such as cellulose, dextrans, and polyhyaluronic acid. Synthetic polymers are generally preferred due to the better characterization of degradation and release profiles. Representative synthetic polymers include polyphosphazenes, polyvinyl alcohols), polyamides, polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Examples of suitable polyacrylates include pory(methyl-methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate). Synthetically modified natural polymers include cellulose derivatives such as alkyl celluloses, hydroxyalkyl celluloses, cellulose ethers, cellulose esters, and nitrocelluloses. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

The polymers described above can be separately characterized as biodegradable, non-biodegradable, and bioadhesive polymers, as discussed in more detail below. Representative synthetic degradable polymers include polyhydroxy acids such as polylactides, polyglycolides and copolymers thereof, poly(ethylene terephthalate), poly(butic acid), poly(valeric acid), poly(lactide-co-caprolactone), polyanhydrides, polyorthoesters and blends and copolymers thereof. Representative natural biodegradable polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers. In general, these materials degrade either by enzymatic hydrolysis or exposure to water in vivo, by surface or bulk erosion. Examples of non-biodegradable polymers include ethylene vinyl acetate, poly(meth)acrylic acid, polyamides, polyethylene, polypropylene, polystyrene, polyvinyl chloride, polyvinylphenol, and copolymers and mixtures thereof. Hydrophilic polymers and hydrogels tend to have bioadhesive properties. Hydrophilic polymers that contain carboxylic groups (e.g., poly[acrylic acid]) tend to exhibit the best bioadhesive properties. Polymers with the highest concentrations of carboxylic groups are preferred when bioadhesiveness on soft tissues is desired. Various cellulose derivatives, such as sodium alginate, carboxymethylcellulose, hydroxymethylcellulose and methylcellulose also have bioadhesive properties. Some of these bioadhesive materials are water-soluble, while others are hydrogels. Polymers such as hydroxypropylmethylcellulose acetate succinate (HPMCAS), cellulose acetate trimellitate (CAT), cellulose acetate phthalate (CAP), hydroxypropylcellulose acetate phthalate (HPCAP), hydroxypropylmethylcellulose acetate phthalate (HPMCAP), and methylcellulose acetate phthalate (MCAP) may be utilized to enhance the bioavailability of hypoglycemic agent and/or copper antagonist with which they are complexed. Rapidly bioerodible polymers such as poly(lactide-co-glycolide), polyanhydrides, and polyorthoesters, whose carboxylic groups are exposed on the external surface as their smooth surface erodes, can also be used for bioadhesive hypoglycemic agent/copper chelator delivery systems. In addition, polymers containing labile bonds, such as polyanhydrides and polyesters, are well known for their hydrolytic reactivity. Their hydrolytic degradation rates can generally be altered by simple changes in the polymer backbone. Upon degradation, these materials also expose carboxylic groups on their external surface, and accordingly, these can also be used for bioadhesive delivery systems for the inhibitors of MST1/2 protein kinases.

Other agents that may enhance bioavailability or absorption of the MST1/2 inhibitors can act by facilitating or inhibiting transport across the intestinal mucosa. For example, blood flow in the stomach and intestine is a factor in determining intestinal drug absorption and drug bioavailability, so that agents that increase blood flow, such as vasodilators, may increase the rate of absorption of orally administered inhibitors of MST1/2 protein kinases by increasing the blood flow to the gastrointestinal tract. Vasodilators have been used in combination with other drugs. For example, in EPO Publication EP0106335, the use of a coronary vasodilator, diltiazem, is reported to increase oral bioavailability of drugs which have an absolute bioavailability of not more than 20%, such as adrenergic beta-blocking agents (e.g., propranolol), catecholamines (e.g., dopamine), and benzodiazepine derivatives (e.g., diazepam).

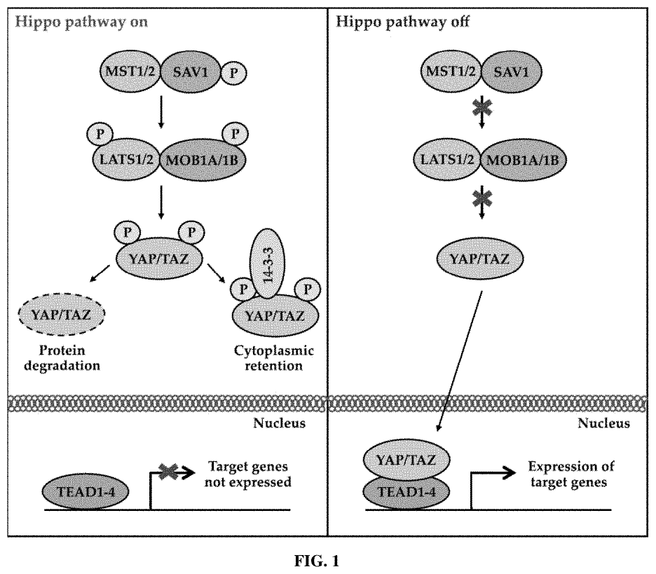

The inhibitors of MST1/2 protein kinases, optionally their salts, prodrugs, or in a pharmaceutical composition as described herein, are administered to a subject in a prophylactically effective amount to prevent, or in a therapeutically effective amount to treat, a disease or disorder associated with tissue damages. The inhibitors of MST1/2 protein kinases of the present invention promote both cell protection by inhibition of cell apoptosis and tissue regeneration by stimulating cell proliferation. See FIG. 1.

Specifically, MST1/2 protein kinases activation induces cell apoptosis through multiple mechanisms: (1) phosphorylating Beclin1 to inhibit autophagy but promote apoptosis (Nature Medicine, 19:1367-1368, 2013); (2) translocating its C terminus into the nucleus to phosphorylate H2B to induce apoptosis (Nature Medicine, 20:385-397, 2014); (3) inhibiting YAP/TAZ/TEAD1-4 signal that normally induces antiapoptotic gene expression. Therefore, inhibiting MST1/2 protein kinases leads to the inhibition of cell apoptosis.

In addition, YAP/TAZ/TEAD1-4 is inhibited when MST1/2 protein kinases are activated. Inhibiting MST1/2 protein kinases will activate YAP/TAZ/TEAD1-4, which normally leads to cell proliferation. As such, inhibiting MST1/2 protein kinases promotes cell proliferation and tissue regeneration.

Further, the inhibition of MST1/2 protein kinases can also reduce tissue inflammation via decreasing endothelial expression of VCAM and other inflammatory cytokines.

Accordingly, the present invention uses the inhibitors of MST1/2 protein kinases to prevent or treat a disease or disorder associated with tissue damages by reducing cell apoptosis, inducing tissue regeneration, and/or inhibiting inflammation. As such, the inhibitors of MST1/2 protein kinases may act on multiple mechanisms to treat these diseases and disorders. There are at least five types of diseases and disorders associated with tissue damages.

The first type of diseases and disorders have tissue damages resulted from trauma to the brain (ischemic stroke, blunt trauma, subarachnoid hemorrhage), spinal cord (ischemia, blunt force trauma), peripheral nerves (sciatic nerve injury, diabetic neuropathy, carpal tunnel syndrome), retinal (macular edema, diabetic retinopathy, glaucoma), and heart (myocardial infarct, chronic heart failure). In a preferred embodiment, the first type of diseases and disorders are ischemic stroke and acute myocardial infarct (hear attack). Stroke includes both focal and global ischemia, as well as transient cerebral ischemic attacks and other cerebral vascular problems accompanied by cerebral ischemia.

The second type of diseases and disorders have tissue damages resulted from an organ failure. For example, diabetes mellitus type I or II, nephrosis, fatty liver diseases, failure of gonads, pancreas, kidney, heart, lung, liver and bowel. In a preferred embodiment, the second type of diseases and disorders are diabetes mellitus type I or II, fatty liver diseases, and heart failure.

The third type of diseases and disorders have tissue damages resulted from exposure to a toxic agent, such chemotherapeutic agents, chemical agents, radiation agents. The toxic agent can cause tissue damages to many organs, for example, organ failure. For example, liver may be damaged by consumption of alcohol and heart may be damaged (cardic injury) by chemotherapeutic agents. Nephrotoxicity may be induced by exposure to a contrast imaging agent. Further, exposure to radiation causes extensive tissue damages to many organs. Many pharmaceutical used to treat other diseases may have side effects of causing tissue damages in some organs, such as liver toxicity and renal toxicity. In a preferred embodiment, the third type of diseases and disorders are cardiac injury induced by a chemotherapeutic agent (e.g., doxorubicin), acute liver damage induced by alcohol, and nephrotoxicity induced by a contrast imaging agent.

The fourth type of diseases and disorders have tissue damages resulted from inflammatory diseases, including sepsis, inflammatory bowel diseases, Crohn's disease, ulcerative colitis, ileitis, and enteritis, acute nephritis, and other inflammatory diseases (either acute or chronic). In a preferred embodiment, the fourth type of diseases and disorders are sepsis, inflammatory bowel disease, Crohn's disease, ulcerative colitis, and acute nephritis.

The fifth type of diseases and disorders have tissue damages resulted from degenerative diseases including muscular dystrophies, myotonic dystrophy, neurodegenerative diseases. Some examples of neurodegenerative diseases include age-related loss of cognitive function and senile dementia, chronic seizure disorders, Alzheimer's disease, Parkinson's disease, dementia, memory loss, amyotrophic lateral sclerosis, multiple sclerosis, tuberous sclerosis, Wilson's disease, cerebral and progressive supranuclear palsy, Guam disease, Lewy body dementia, prion diseases, such as spongiform encephalopathies, e.g., Creutzfeldt-Jakob disease, Huntington's disease, Freidrich's ataxia and other ataxias. Degenerative diseases also include diseases caused by excessive bone loss or cartilage or matrix degradation such as: osteoporosis, glucocorticoid induced osteoporosis, Paget's disease, abnormally increased bone turnover, periodontal disease, gingivitis, tooth loss, bone fractures, arthritis, rheumatoid arthritis, osteoarthritis, periprosthetic osteolysis, osteogenesis imperfecta, or metastatic bone disease.

The inhibitors of MST1/2 protein kinases or pharmaceutical compositions of the present invention may be effectively administered at a plurality of times associated with tissue damage, including prior to actual damage, during various stages of damage development, and/or after damage has occurred. Specifically, the inhibitors of MST1/2 protein kinases or pharmaceutical compositions can be used prophylactically to protect a subject against the development of tissue damage, such as from one minute to about 24 hours, or from about five minutes to about 10 hours, or from about five minutes to about five hours prior to onset of the tissue damage. The inhibitors of MST1/2 protein kinases or pharmaceutical compositions can also be used after the development of tissue damage for a short period of time, such as from one minute to about 24 hours, or from about five minutes to about 10 hours, or from about five minutes to about five hours.

The inhibitors of MST1/2 protein kinases or pharmaceutical compositions can also be used after the development of tissue damage for an extended period of time, such as days to weeks, months, or even years to prevent continuing primary or secondary injury. Finally, the inhibitors of MST1/2 protein kinases or pharmaceutical compositions can be used after the tissue damage has been alleviated (or there is reason to believe it has been improved), in an attempt to prevent the onset of a similar tissue damage in the future.

To treat a disease or disorder associated with tissue damages, the inhibitors of MST1/2 protein kinases may be administered through intravenous administration (by injection or infusion or drip), subcutaneous injection, sublingual administration, or oral administration to a subject at a dose in the range of from about 1 to about 10 mg/kg bodyweight. In some embodiments, the dose may be from at least about 2, 3, 4, 5, 6, 7, 8, and 9 mg/kg bodyweight up to about 10 mg/kg bodyweight. In some embodiments, the dose may be from about 1 mg/kg bodyweight up to about 2, 3, 4, 5, 6, 7, 8, and 9 mg/kg bodyweight. In some embodiments, the dose may be from any one of about 2, 3, 4, 5, 6, 7, 8, and 9 mg/kg bodyweight to any one of about 2, 3, 4, 5, 6, 7, 8, and 9 mg/kg bodyweight.

In some embodiments, the dosage of the inhibitors of MST1/2 protein kinases may be in the range of from about 0.1 to about 100 mg/kg bodyweight. In some embodiments, the dosage of the inhibitors of MST1/2 protein kinases may be in the range of from about 0.1 to about 10 mg/kg bodyweight. In some embodiments, the dosage of the inhibitors of MST1/2 protein kinases may be in the range of from about 1 to about 100 mg/kg bodyweight. In some embodiments, the dosage of the inhibitors of MST1/2 protein kinases may be in the range of from about 10 to about s100 mg/kg bodyweight. In some embodiments, the dosage of the inhibitors of MST1/2 protein kinases may be in the range of from any one of about 10, 20, 30, 40, 50, 60, 70, 80, 90 mg/kg bodyweight to any one of about 20, 30, 40, 50, 60, 70, 80, 90, 100 mg/kg bodyweight.

In the embodiment where the inhibitors of MST1/2 protein kinases are administered by intravenous infusion or intravenous injection or intravenous drip, the entire dose of the inhibitors may be administered for a period of from about 0.3 hour to about 12 hours, or from about 0.5 hour to about 10 hours, or from about 1 hour to about 8 hours, or from 1 hour to about 6 hours, or from about 1 hour to about 4 hours.

In some embodiments, the inhibitors of MST1/2 protein kinases may be administered before a tissue injury is expected to occur, such as at the time when angina occurs in ischemic heart disease patients, or before a cancer patient receiving cardiotoxic chemotherapy agents. In some embodiments, the inhibitors of MST1/2 protein kinases may be administered at the time with injury inducers when e a tissue injury is expected to occur, such as a cancer patient receiving cardiotoxic chemotherapy agents. In some other embodiments, the inhibitors of MST1/2 protein kinases may be administered after tissue injury, such as after stroke or acute liver injury, or after inflammatory diseases have developed.

The inhibitors of MST1/2 protein kinases may be administered one time or twice before the tissue injury is expected to occur. After the tissue injury has occurred, the inhibitors of MST1/2 protein kinases may be administered four times per day, three times per day, twice per day, or once every 1, 2, 3, 4, 5, 6, 7, 8, 9, 10, 11, 12, 13, 14, 15, 16, 17, 18, 19, or 20 day(s), or at other schedules, such as once every a few days.

In some embodiments, the inhibitors of MST1/2 protein kinases are administered intravenously (by injection or infusion or drip) or subcutaneously to a subject having or potentially developing tissue injury due to heart attack, cardiac injury induced by chemotherapy, stroke, acute liver damage induced by alcohols, acute nephritis, or nephrotoxicity induced by contrast imaging agent.

In some embodiments, the inhibitors of MST1/2 protein kinases are administered sublingually or orally to a subject having or potentially developing tissue injury due to heart failure, fatty liver diseases, sepsis, inflammatory diseases, and diabetes.

In some embodiments, the inhibitors of MST1/2 protein kinases may be co-administered (either simultaneously or in separate sequential administrations) with one or more further active ingredients known to treat degenerative diseases/tissue injuries. Preferably, if the administration is not simultaneous, the inhibitors of MST1/2 protein kinases and the further active ingredients are administered in a close time proximity to each other, within half an hour, or 1 hour, or 2 hours, or 4 hours, or 6 hours, or 8 hours, or 10 hours. Furthermore, the inhibitors of MST1/2 protein kinases and the further active ingredients may be administered in the same dosage form or different dosage forms, e.g. one compound may be administered intravenously and another compound may be administered orally.

Examples of further active ingredients for use in combination with the inhibitors of MST1/2 protein kinases include but are not limited to: chemoprotective or myeloprotective agents such as G-CSF, BB10010 (Clemons et al., Breast Cancer Res. Treatment, 57, 127, 1999), amifostine (Ethyol) (Fetscher et al., Current Opinion in Hemat., 7, 255-60, 2000), SCF, IL-11, MCP-4, IL-1-beta, AcSDKP (Gaudron et al., Stem Cells, 17,100-6, 1999), TNF-a, TGF-b, MIP-1a (Egger et al., Bone Marrow Transpl., 22 (Suppl. 2), 34-35, 1998), and other molecules identified as having anti-apoptotic, survival or proliferative properties.

Additional examples of further active ingredients for use in combination with the inhibitors of MST1/2 protein kinases include but are not limited to: stem cell, megakaryocyte, neutrophil mobilizers such as chemotherapeutic agents (i.e., cytoxan, etoposide, cisplatin, Ballestrero A. et al., Oncology, 59:7-13, 2000), chemokines, IL-8, Gro-beta (King, A. G. et al. J. Immun., 164:3774-82, 2000), receptor agonist or antagonist antibodies, small molecule cytokine or receptor agonists or antagonists, SCF, Flt3 ligand, adhesion molecule inhibitors or antibodies such as: anti-VLA-4 (Kikuta T. et al., Exp. Hemat., 28:311-7, 2000) or anti-CD44 (Vermeulen M. et al., Blood, 92:894-900, 1998), cytokine/chemokine/interleukin or receptor agonist or antagonist antibodies, MCP-4 (Berkhout T A., et al., J. Biol. Chem., 272:16404-16413, 1997; Uguccioni M. et al., J. Exp. Med., 183:2379-2384, 1996).

In some embodiments, further active ingredients for use in combination with the inhibitors of MST1/2 protein kinases also include, but are not limited to, carbamates (pyridostigmine, physostigmine, aminostigmine, neostigmine, synostigmine, Epastigmine, Mobam, decarbofuran), anticholingerics (trihexyphenidyle, benactyzine, Biperidene, Scopolamine, aprophen, atropine, hyoscin, adiphenine, Caramiphen, pentmethonium, Mecamylamine, Trihexyphenidyle) PANPAL, aminophenols (eseroline), organophosphates (TEPP, Paraxon, Ethyl-4-nitrophenylphosphate), tacrine, 7-MEO-TA, huperzine A, Cholinesterases (BuChE, AChE, triesterase, paraoxonase), oximes/reactivators (HI-6, PAM, Obidoxime, Trimedoxime, Methoxime, Hlo-7, BI-6, K048, K033, pralidoxime chloride (2-PAM Cl), P2S, TMB4, 2-PAMI), Suramine, Benzodiazepines, tubocurine, Memantine, Procyclidine, Nimodipin, Clonidine, pralidoxime, diazepam, enkephalins, phenylmethylsulfonyl fluoride, natrium bicarbonate, vitamin E analogs (α-tocopherol succinate, γ-tocotrienol), superoxide dismutase/catalase mimic (EUK189), selenium, benzyl styryl sulfone, truncated flagellin, statins, genistein, galantamine, hypothermia, 5-androstenediol, CpG-oligodeoxynucleotides, antimicrobials, stem cell transplants, amifostine, Tempol, isoflavones, benzylsulfone analogs, GM-CSF, G-CSF, potassium iodide, aluminum hydroxide, Prussian blue, chelating agents (diethylenetriaminepentaacetate (Ca-DTPA), zinc diethylenetriaminepentaacetate (Zn-DTPA)), keratinocyte growth factor, intestinal peptide hormones, beta glucan, octreotide, pentoxifylline, angiotensin converting enzyme inhibitors, angiotensin II receptor blockers, methemoglobin formers (amyl nitrite, sodium nitrite), sodium thiosulfate, cobalt compounds (hydroxycobalamin (Vitamin B12a), toxoids, antitoxins, vaccines, passive antibodies, chemotherapeutic agents including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel; Radiation: α-radiation; Alkylating agents; Nitrogen mustards: cyclophosphamide, Ifosfamide trofosfamide, Chlorambucil; Nitrosoureas: carmustine (BCNU), Lomustine (CCNU), Alkylsulphonates busulfan, Treosulfan; Triazenes: Dacarbazine; Platinum containing compounds: Cisplatin carboplatin, Plant Alkaloids; Vinca alkaloids: vincristine, Vinblastine, Vindesine, Vinorelbine; Taxoids: paclitaxel, Docetaxol; DNA Topoisomerase Inhibitors Epipodophyllins: etoposide, Teniposide, Topotecan, 9-aminocamptothecin irinotecan (Campto®), crisnatol; Mytomycins: Mytomycin C, Mytomycin C; Anti-metabolites, Anti-folates: DHFR inhibitors: methotrexate, Trimetrexate; IMP dehydrogenase Inhibitors: mycophenolic acid, Tiazofurin, Ribavirin EICAR; Ribonuclotide reductase inhibitors: hydroxyurea; deferoxamine; Pyrimidine analogs: Uracil analogs, 5-Fluorouracil, Floxuridine, Doxifluridine, Ratitrexed; Cytosine analogs: cytarabine (ara C) Cytosine arabinoside fludarabine; Purine analogs: mercaptopurine, Thioguanine; Hormonal therapies; Receptor antagonists: Anti-estrogens, Tamoxifen, Raloxifene megestrol; LHRH agonists: goserelin, Leuprolide acetate; Anti-androgens: flutamide, bicalutamide; Retinoids/Deltoids Vitamin D3 analogs: EB 1089, CB 1093, KH 1060; Photodyamic therapies: vertoporfin (BPD-MA), Phthalocyanine photosensitizer, Pc4 Demethoxy-hypocrellin A (2BA-2-DMHA) Cytokines: Interferon-α, Interferon-γ, Tumor necrosis factor; Isoprenylation inhibitors: Lovastatin; Dopaminergic neurotoxins: 1-methyl-4-phenylpyridinium ion; Cell cycle inhibitors: staurosporine; Actinomycins: Actinomycin D, Dactinomycin; Bleomycins: bleomycin A2, Bleomycin B2, Peplomycin; Anthracyclines: daunorubicin, Doxorubicin (adriamycin), Idarubicin, Epirubicin, Pirarubicin, Zorubicin, Mitoxantrone; MDR inhibitors: verapamil; Ca²⁺ ATPase inhibitors: thapsigargin; TNF-α inhibitors/thalidomide angiogenesis inhibitors 3-(3,4-dimethoxy-phenyl)-3-(1-oxo-1, 3-dihydro-isoindol-2-yl)-propionamide (SelCIDs™) ImiDs™, Revlimid™, Actimid™. In another aspect of the present invention, a pharmaceutical composition according to the present invention may include an MST1/2 inhibitor in a formulation with at least one small molecule that exhibits tissue protective functionality. Suitable small molecules include, but are not limited to, steroids (e.g., lazaroids and glucocorticoids), antioxidants (e.g., coenzyme Q₁₀, alpha lipoic acid, and NADH), anticatabolic enzymes (e.g., glutathione peroxidase, superoxide dimutase, catalase, synthetic catalytic scavengers, as well as mimetics), indole derivatives (e.g., indoleamines, carbazoles, and carbolines), nitric acid neutralizing agents, adenosine/adenosine agonists, phytochemicals (flavanoids), herbal extracts (ginko biloba and turmeric), vitamins (vitamins A, E, and C), oxidase electron acceptor inhibitors (e.g., xanthine oxidase electron inhibitors), minerals (e.g., copper, zinc, and magnesium), non-steriodal anti-inflammatory drugs (e.g., aspirin, naproxen, and ibuprofen), and combinations thereof. Additionally agents including, but not limited to, anti-inflammatory agents (e.g., corticosteroids, prednisone and hydrocortisone), glucocorticoids, steroids, non-steriodal anti-inflammatory drugs (e.g., aspirin, ibuprofen, diclofenac, and COX-2 inhibitors), beta-agonists, anticholinergic agents and methyl xanthines), immunomodulatory agents (e.g., small organic molecules, T cell receptor modulators, cytokine receptor modulators, T-cell depleting agents, cytokine antagonists, monokine antagonists, lymphocyte inhibitors, or anti-cancer agents), gold injections, sulphasalazine, penicillamine, anti-angiogenic agents (e.g., angiostatin), TNF-α antagonists (e.g., anti-TNFα antibodies), and endostatin), dapsone, psoralens (e.g., methoxalen and trioxsalen), anti-malarial agents (e.g., hydroxychloroquine), anti-viral agents, anti-histamines and antibiotics (e.g., erythromycin and penicillin).

In some embodiments, the inhibitors of MST1/2 protein kinases may be administered in conjunction with methods of treatment such as chemotherapy, radiation therapy (x-ray radiation, high-energy megavoltage (radiation of greater that 1 MeV energy), electron beam, orthovoltage x-ray radiation, gamma-ray emitting radioisotopes (radioactive isotopes of radium, cobalt and other elements)), hyperbaric chambers, heart bypass machine, angioplasty, hypothermia, surgery, angioplasty, etc. to to achieve additive, synergistic or offsetting (to counteract side effects of the therapeutic method) benefits of the effects of the inhibitors of MST1/2 protein kinases. As an example, in a specific embodiment, the inhibitors of MST1/2 protein kinases can be administered to a patient that has undergone chemotherapy or radiation therapy. In another specific embodiment, a chemotherapeutic agent or radiation therapy is administered prior or subsequent to administration of the inhibitors of MST1/2 protein kinases, preferably at least an hour, two hours, three hours, five hours, 12 hours, a day, two days, three days, or a week. The inhibitors of MST1/2 protein kinases may be administered as a supplement to chemotherapy or radiation therapy where the chemotherapy or the radiation therapy has proven or may prove too toxic, e.g., results in unacceptable or unbearable side effects, for the patient being treated. The inhibitors of MST1/2 protein kinases may be administered, simultaneously with or following treatment with chemotherapy or radiation in an effort to prevent or ameliorate the toxic side effects of the treatment method.

### Exemplary Diseases and Disorders

In verifying applications of the inhibitors of MST1/2 protein kinases for these exemplary diseases and disorders, six exemplary compounds were used:

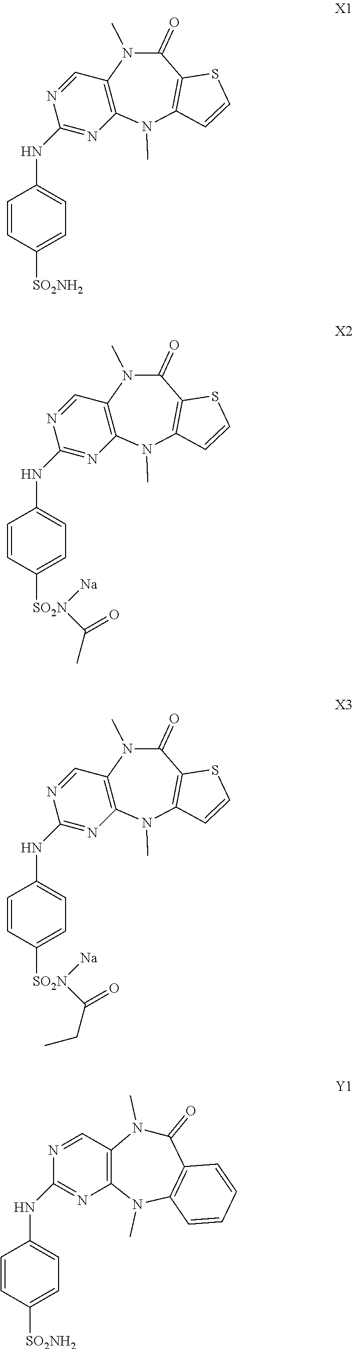

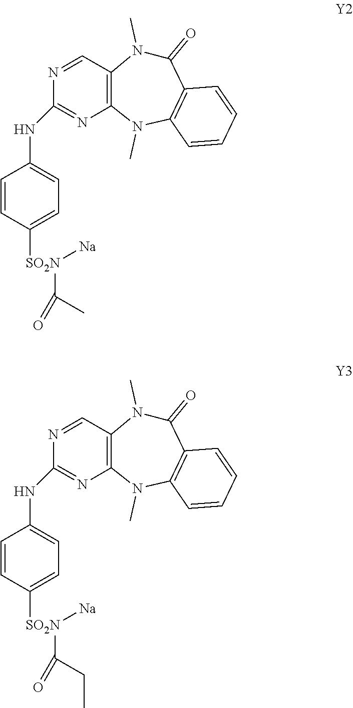

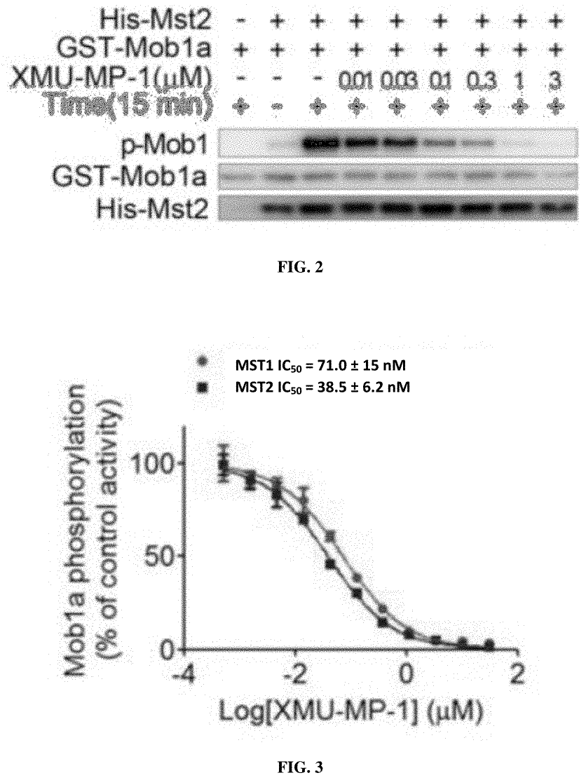

The activity of these compounds in preventing phosphorylation of Mob1 is an important event in preventing cell apoptosis and inducing tissue/organ regeneration (FIG. 1). The compounds show dose-dependent inhibition of phosphorylation of Mob1 (FIG. 2). The IC50 in inhibiting Mob1 phosphorylation through MST1 and MST2 is in the nano-molar range. For example, one of these compounds has IC50 at 71.0±15 nM for MST1 and 38.5±6.2 nM for MST2 (FIG. 3). Thus, these six compounds have excellent activity in inhibiting MST1/2 and suppression of the Hippo pathway, which lead to expression of the target genes, including anti-apoptosis genes, cell division related genes and genes involved in inhibition of cell differentiation.

### A. Cardio Protection

The cardiomyocytes were isolated from neonatal rats and cultured using the following protocol:
1. Pre-sterilize forceps, scissors, and beaker with a stir bar. Also prepare sterilized dH₂O ahead of time.
2. Coating 12 well of Plate by SureCoat solution and leave at 37° C. till use.
3. Prepare wash buffer: Prepare 2 of 100 mm dishes with 1× cold PBS and 1 of 50 ml of tube with 1× cold PBS. Keep them cold.
4. Prepare D2: 28 ml of dH₂O+20 ml of Cellutron D2+2 ml of EC; filter to sterilize. This is to be kept at room temperature.
5. Prepare 10 ml of FBS (5 ml is enough for one isolation).
6. Sterilize neonates with 70% EtOH; Collect hearts into one dish with cold PBS, and remove atria, and transfer hearts into the 2nd dish with cold PBS. Wash multiple times with the 50 ml cold PBS (this can prevent contamination later on). Transfer the hearts into enzymes.
7. Digestion is done at 37° C. in a small beaker with a stirrer bar (pre-sterilized) and stirrer at 150 rpm). Each digestion is 9 min/5 ml (total 10 digestions), followed by pipetting to break down cells. The cell is to be pelleted at 1000 g for 1 min. Then cells are collected into the tube of FBS until the end.
8. Pellet cells down and remove FBS, re-suspend in DMEM 11995+10% FBS+P/S. Use 50 ml for 10 neonatal rat hearts.
9. Filter through a cell strainer is to remove the clumps.
10. Count cells with Trypan blue before pre-plating to get an idea how many live cells there would be, to get an idea how many plates should be coated (the actual cell # will be smaller, only ˜⅔ after pre-plating at 37° C. for 1˜1.5 hrs).
11. Pre-plate in uncoated tissue culture dish (3 of 100 mm dishes/per 10 neonatal rat hearts) in 370 C CO₂ incubator for 1˜1.5 hrs.
12. Move unattached cells to sterile 50-mL tubes. Count live cell density.
13. Plating: Adjust cells with culture medium (DMEM+10% FBS+P/S)+100 μM BrDU (To block fibroblasts from growing up) to 10⁶ cell/mL, and plate cells in 6-well cell culture plates with 2×10⁶ cells/well.
14. Next day, wash with DMEM with PPennecilin/Streptomycin without FBS to remove dead cells (around 2˜3 times; check cell under microscope).
15. For cell live/death assay, add DMEM with 10% FBS and 1% P/S or 1% FBS and 1% P/S, and several concentrations of treatments according to experimental design.
16. For EdU assay use DMEM with 1% FBS and 1% P/S. Add 0.5 μM EdU per well except adding different concentration of compounds.
17. Medium and treatment need to be changed every day to prevent contamination if cells need culture more than one day. For EdU assay, change medium with same compounds and EdU and culture for another 24 hr.

The isolated rat cardiomyocytes (neonatal rat cardiomyocytes or NRCM) were subjected to a live/died cell assay. Specifically, the isolated rat cardiomyocytes were incubated with different concentration of compounds (X1, X2, Y1 or Y2) at 37° C. and 5% CO₂ for 1 hr. Chemical agents that are toxic to the cells were then added to the incubated cells, including H₂O₂, isoproterenol (ISO), and doxorubicin (DOX). The cardiomyocytes with the compounds and the chemical agents were further incubated at 370 C and 5% CO₂O/N for 24 hrs. The cardiomyocytes were stained with 0.4% Trypan Blue at room temperature for 15 min to estimate the percentage of dead cells which were stained blue by Trypan blue (cell death rate). Images of the stained cells were taken for 3-5 random fields per well for analysis of the death rate.

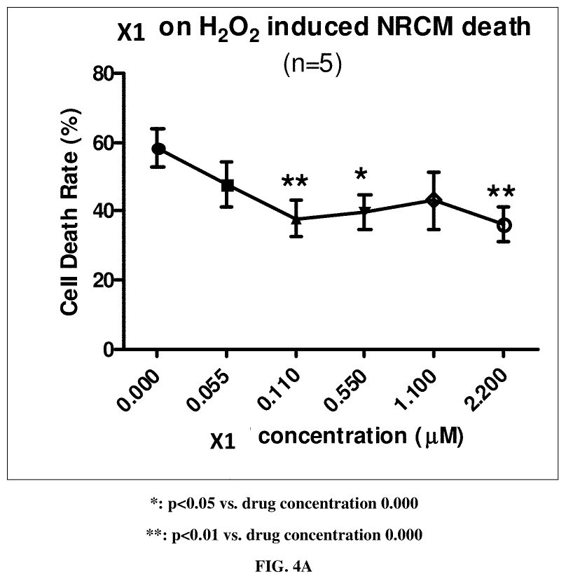

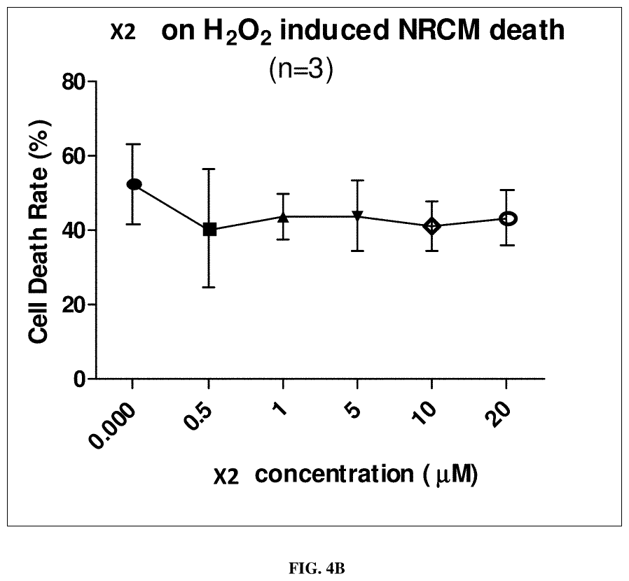

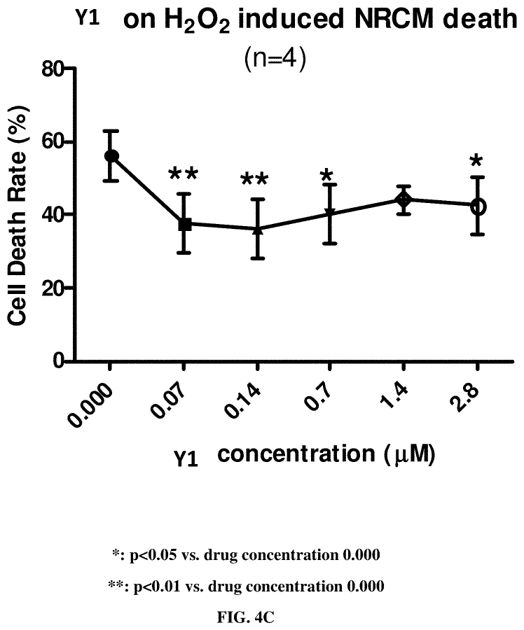

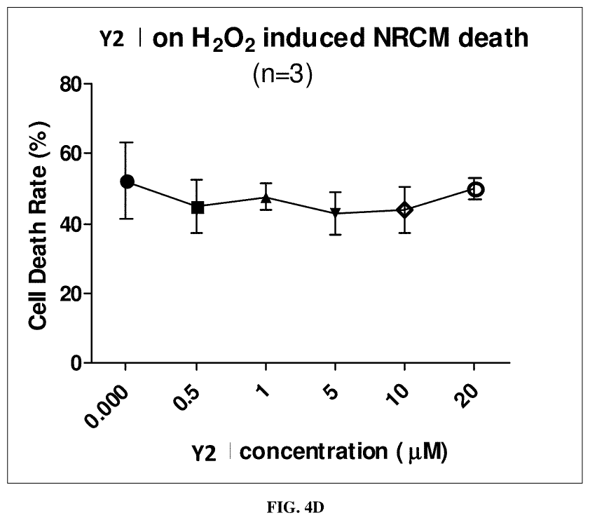

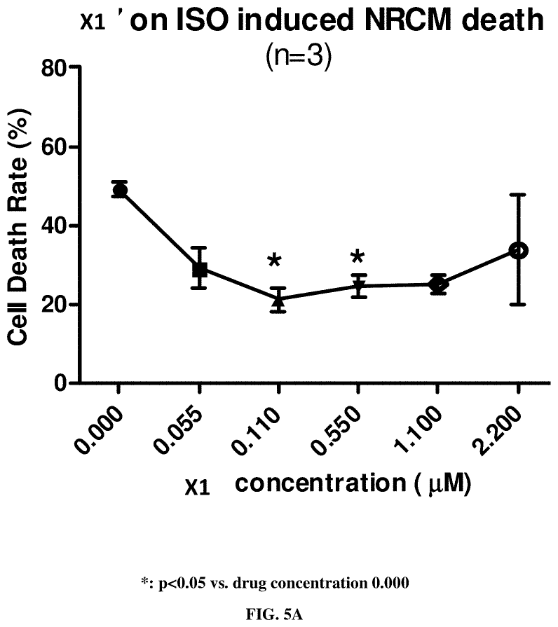

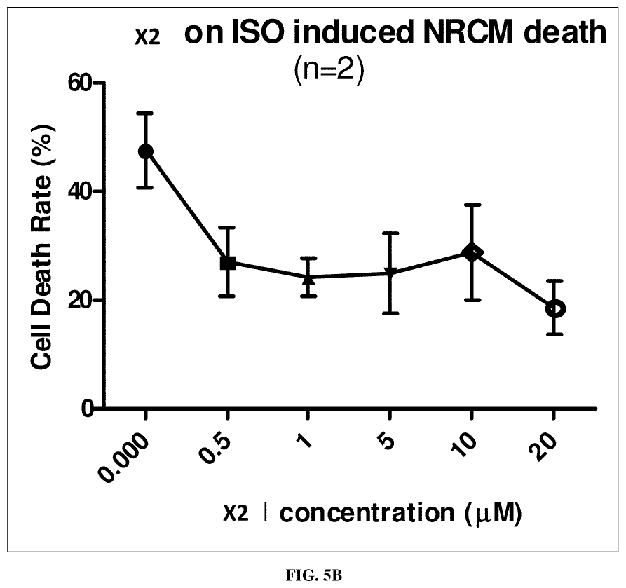

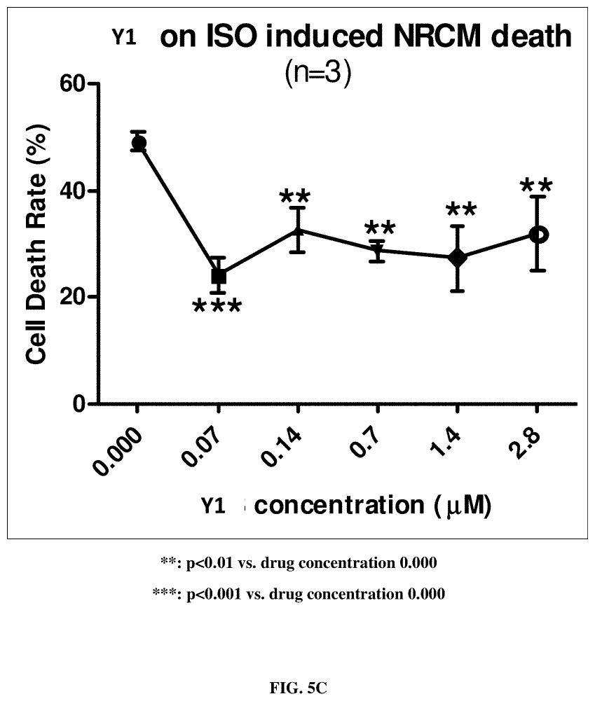

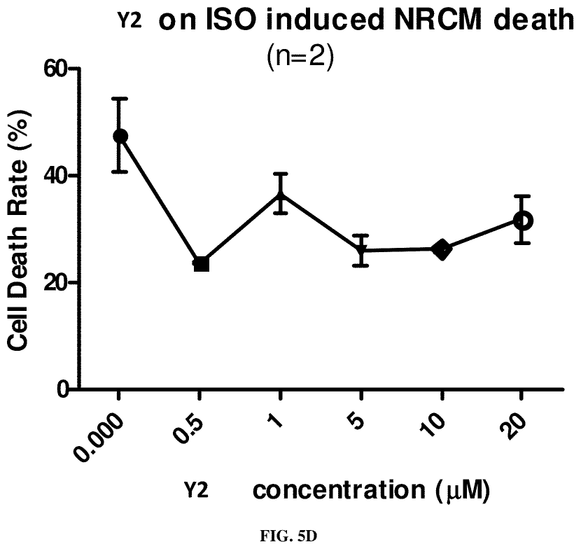

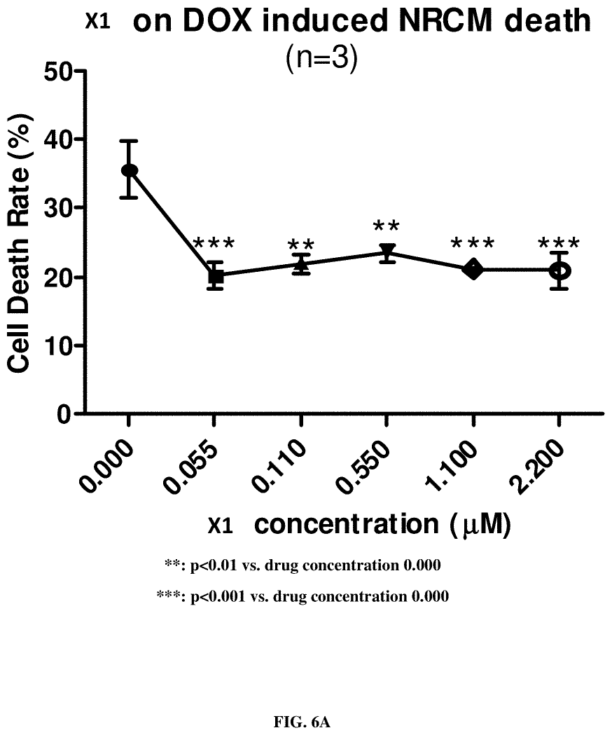

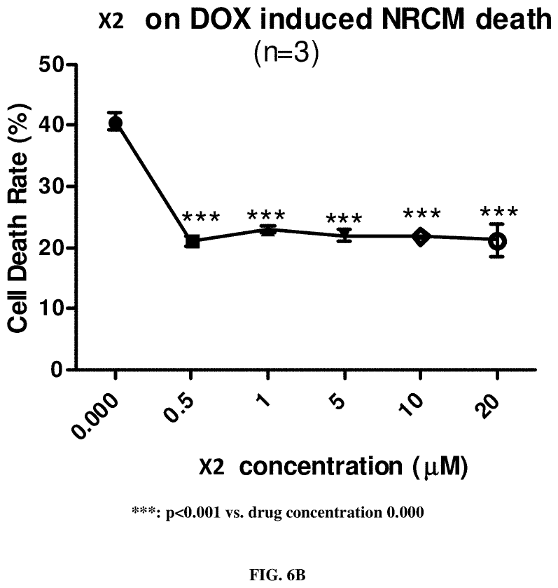

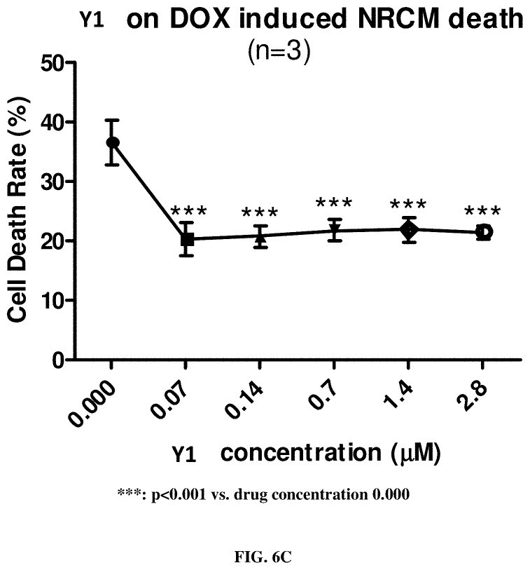

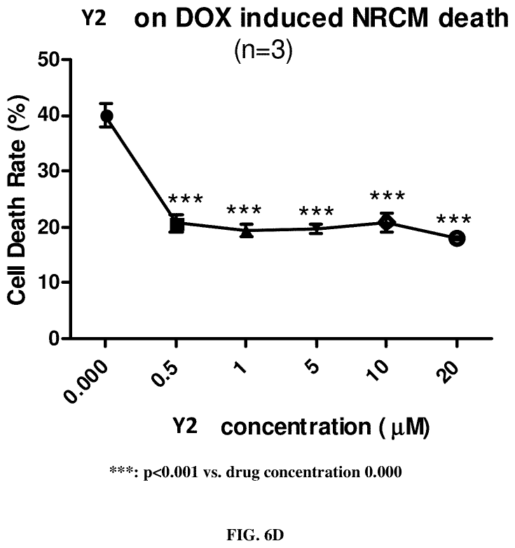

The MST1/2 inhibitors were found to significantly reduce death rate of cardiomyocytes induced by 20 μM H₂O₂(FIGS. 4A-4D), by 100 μM ISO (FIGS. 5A-5D) or by 0.2 μM DOX (FIGS. 6A-6D). These experiments indicated that the inhibitors of MST1/2 protein kinases are effective in preventing or treating injuries to cardiomyocytes induced by these toxic agents.

The apoptosis in cardiomyocytes induced by the toxic agents (H₂O₂, ISO and DOX) was measured by the apoptosis detection assay using the DeadEnd™ Fluorometric TUNEL System by the following protocol:
1. Cardiomyocytes were fixed by adding 4% PFA in PBS for 1 hr. at RT after incubated 24 hours.
2. Wash twice by 1× PBS, 5 minutes each time.
3. Drain, and dry the wells completely. Rehydrate with 1×PBS.
4. Permeabilize cells by 0.2% Triton® X-100 in PBS for 5 minutes.
5. Wash cells twice by 1× PBS, 5 minutes each time.
6. Equilibrate: Add 100 μl Equilibration Buffer. Equilibrate at room temperature for 5-10 minutes.
7. Label: Add 50 μl of TdT reaction mix to the cells on an area no larger than 5 square centimeters. Do not allow cells to dry completely. Incubate plate for 60 minutes at 37° C. in a humidified incubator; avoid exposure to light from this step forward.
8. Stop Reaction by adding 2×SSC to well for 15 minutes.
9. Wash cells three times by 1× PBS, 5 minutes each time.
10. Stained nuclei by DAPI.
11. Analyze: Detect localized green fluorescence of apoptotic cells by fluorescence microscopy. Stained nuclei by DAPI will be blue.
12. Randomly take images of 5-10 field per well and save image for analysis.

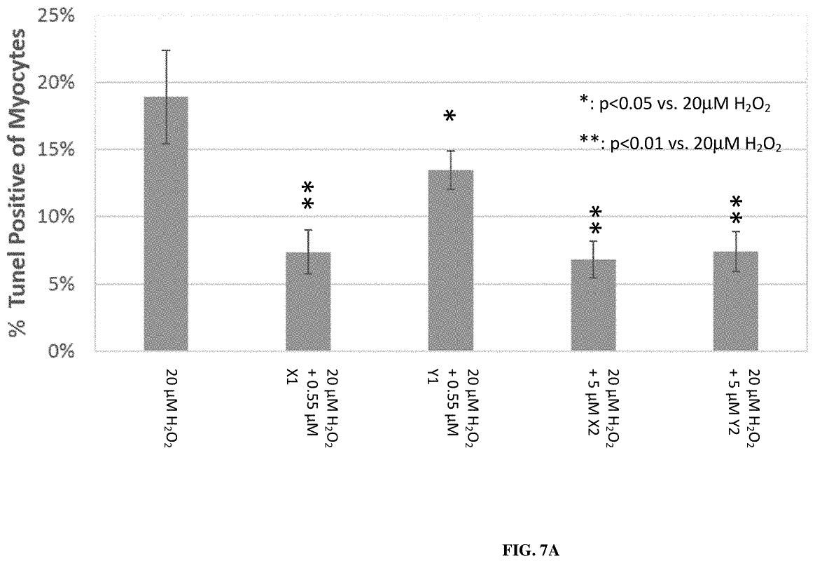

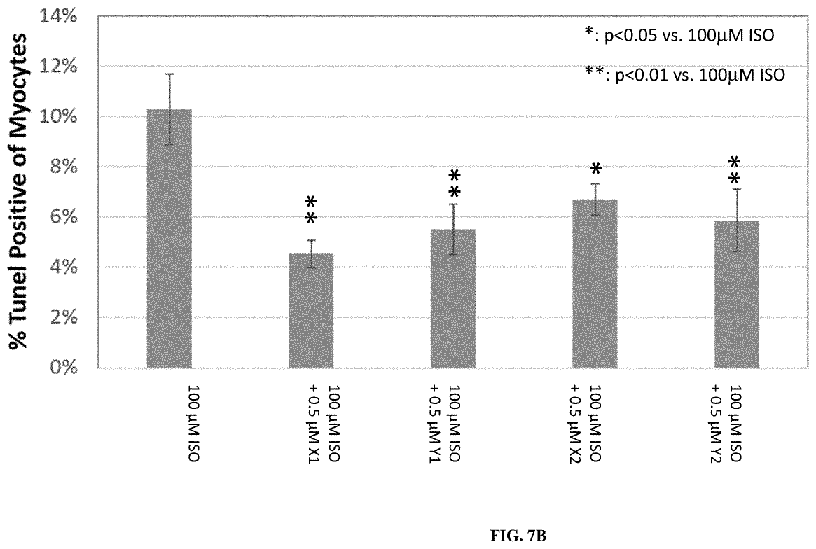

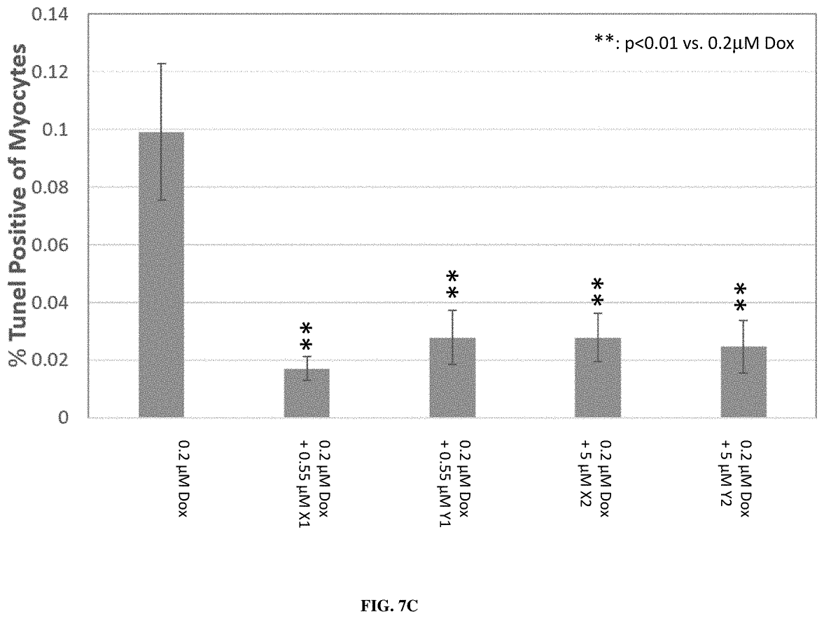

The apoptosis of the cardiomyocytes was determined by percentages of Tunnel positive stain. As shown in FIGS. 7A-7C, the toxic agents (H₂O₂, ISO and DOX) induced apoptosis in the cardiomyocytes. If the cardiomyocytes were pretreated with the inhibitors of MST1/2 protein kinases, the apoptosis was significantly reduced (FIGS. 7A-7C).

In vivo cardio protection in mice by the inhibitors of MST1/2 protein kinases was verified by measuring heart function and atrophy induced by DOX (FIG. 8). Two inhibitors X1 (drug 1) and Y1 (drug 2) were administered to the mice daily by i.p. during the entire study period. After administration of the inhibitors for six days, DOX was administered every two days by i.p. for four times. Blood samples were collected periodically from the mice by ultra-sound. EdU was administered to the mice during the final four days of the study (FIG. 8).

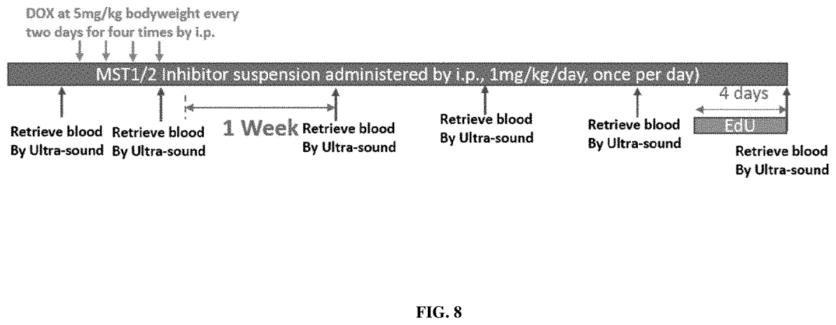

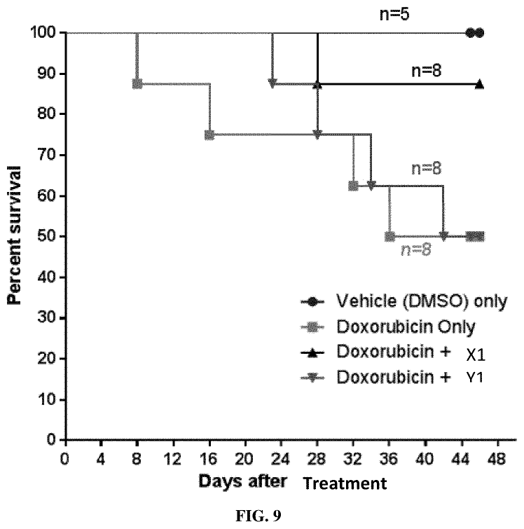

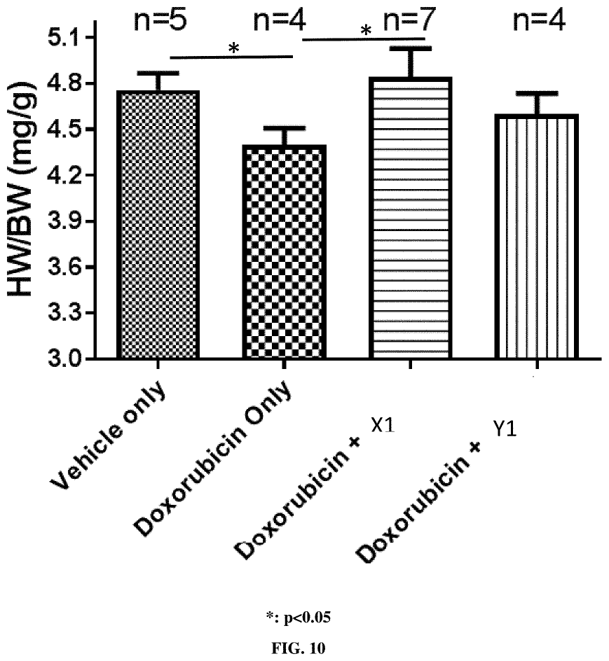

The survival of the mice was summarized in FIG. 9, which showed that the inhibitors of MST1/2 protein kinases can delay death of mice induced by DOX. Further, the mice were disected at the death or at the end of the study and the weight of the hearts was measured. The ratio of heart weight and body weight of the mice (mg/g) was presented in FIG. 10, which showed that the inhibitors of MST1/2 protein kinases can reduce or prevent heart weight loss induced by DOX. This indicated that the inhibitors of MST1/2 protein kinases can reduce death and heart atrophy induced by chemotherapeutic agents such as DOX.

The heart function measured as EF % (ejection fraction) was impaired by DOX over the four-week study period. The inhibitors of MST1/2 protein kinases X1 and Y1 reduced the EF % decrease that was induced by DOX by week four (FIG. 11).

Finally, the cardiomyocytes from the rat hearts were incubated with EdU and DOX, optionally in combination with the inhibitors of MST1/2 protein kinases. The cardiomyocytes were examined for the rate of EdU incorporation into the cardiomyocytes, which indicated heart regeneration (cardiomyocyte divisions). The rate of EdU incorporation was measured by The Click-iT® Plus EdU Imaging Assay, using the following protocol:
1. From cell isolation and culture of step 16, cells were fixed by adding 4% PFA in PBS for 1 hr. at room temperature after incubated 48 hours.
2. Wash twice by 1× PBS, 5 minutes each time.
3. Drain, and dry the wells completely, then circle with immunoEdge pen (to minimize the volume of reagents needed), let it dry. Rehydrate with 1×PBS.
4. Permeabilize cells by 0.2% Triton® X-100 in PBS for 5 minutes.
5. Wash cells twice by 1× PBS, 5 minutes+each time.
6. Blocking: 3% BSA/1×PBS, RT 30 min-1h.
7. Cool down centrifuge to 4 C; spin primary antibody anti-α sarcomeric actin (MA121597, mouse IgM) at 10,000 g for 5 min at 4 C; dilute 1:100 in 3% BSA/1×PBS and add to cover the cells; incubate at 4° C. O/N.
8. Wash cells twice by 1× PBS, 5 minutes each time from overnight incubated with primary antibody. Then continue EdU staining. (No light from here all the way down (fluorescent staining).
9. Prepare 1×Click-iT EdU reaction buffer (component D) working solution by diluting component D 1:10 with dH₂O. Add 2 ml dH₂O to component F to result in 10× stock solution of the Click-iT EdU buffer additive (stored at −20° C. in aliquots) (if it turns brown, it's bad; still good if light brown). Right before use, prepare 1× Click-iT EdU buffer additive by diluting the 10× solution 1:10 in dH₂O (prepare fresh and use right away).
10. Prepare Click-iT Plus reaction cocktail as in the table below in the order as listed (and use within 15 min):

| Reaction components* | 1 |
|---|---|
| 1X Click-iT ® reaction buffer (prepared in step 9) | 440 μL |
| Copper protectant(Component E) | 10 μL |
| Alexa Fluor ® picolyl azide (Component B) | 1.2 μL |
| Reaction buffer additive (prepared in step 9) | 50 μL |
| Total volume | 500 μL |

Component B is fluorescent azides reacting with incorporated EdU in newly synthesized DNA.
11. Add Click-iT reaction cocktail to cover the cells; Incubate for 30 min at RT (no light).
12. Wash clean with 1×PBS.
13. Apply secondary Anti-Mouse IgM antibody in 3% BSA/1×PBS for 1h at RT (A21043 Invitrogen, 568 goat anti-mouse IgM; 1:1000)—add DAPI directly without washing, and incubate for another 10 min. Thorough wash with 1×PBS.
14. Check stain quality and take image by randomly choosing 5-10 field per well and save the images for offline analysis.

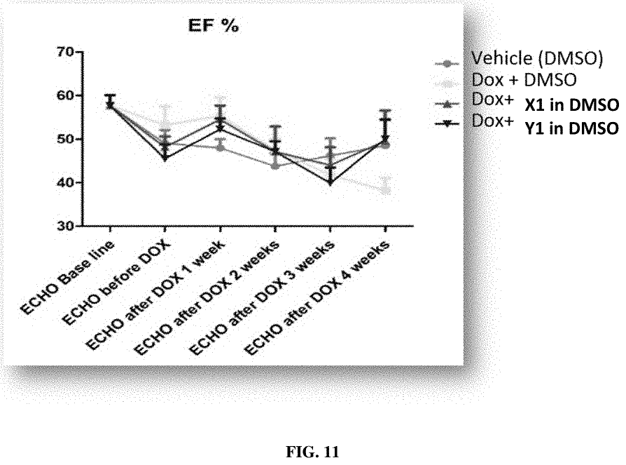

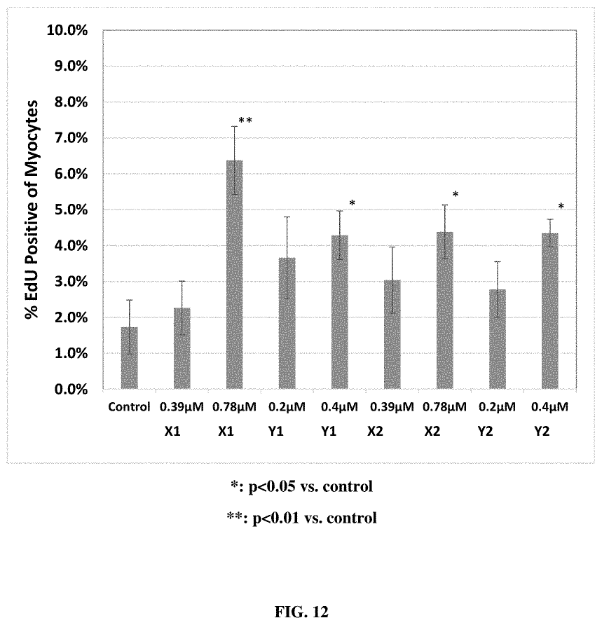

The rate of EdU incorporation into the cardiomyocytes was presented in FIG. 12, which showed that significantly more EdU was incorporated into cardiomyocytes when the inhibitors of MST1/2 protein kinases were present.

Accordingly, these series of experiments verified that the inhibitors of MST1/2 protein kinases of the present invention are effective in preventing or treating heart atrophy induced by toxic agents such as chemotherapeutic agent DOX.

### B. Ischemic Stroke

To verify the protective effect of the inhibitors of MST1/2 protein kinases on the brain injury after ischemic stroke, X1 was administered to mouse cerebral ischemia model induced by transient middle cerebral artery occlusion (MCAO). Briefly, 8-12 week-old C57B1/6 mice (both male and female mice) were intraperitoneal injection with vehicle (DMSO) or 2 mg/kg X1 twice at 24 hours and 3 hours prior to MCAO procedure respectively. Mice were then anesthetized with 2.0% isoflurane, with their body temperature maintained at 37° C. with a heating pad. Following a midline cervical skin incision, the proximal right common carotid artery and the right external carotid artery were ligated. A 6-0 silicon rubber-coated monofilament (6023910PK10; Doccol, Sharon, Mass.) was inserted via the right internal carotid artery to occlude the origin of the right middle cerebral artery. The suture was left in place for 60 minutes to cause ischemia and then removed to allow for reperfusion.

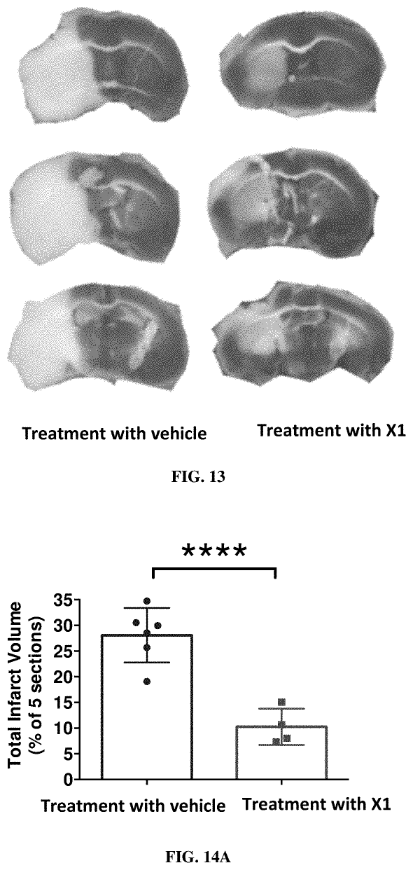

Real-time cerebral blood flow was monitored using a laser speckle contrast imager (PeriCam PSI HR System, Perimed, Sweden) to confirm occlusion of the MCAO and reperfusion after removal of the filament. After 23 hours of reperfusion, mice were then sacrificed and their brains were harvested and sectioned into 1 mm sections. Brain sections were stained with 2% 2,3,5-Triphenyltetrazolium chloride (T8877; Sigma-Aldrich, St. Louis, Mo.) and scanned (HP Scanjet G4010) to allow quantification of infarcted and uninjured brain tissue. Statistical analysis of the data was performed using Prism 6 (GraphPad). Student's t test (mean±standard error of the mean) was used to determine statistical significance. P<0.05 was regarded as statistically significant. As shown in FIGS. 13 and 14A, X1 significantly attenuated the infarct size in the cerebral ischemia model mice.

In addition, neurological function of the MCAO-induced cerebral ischemia model mice was evaluated after treatment with the MST1/2 protein kinases inhibitor X1. Neurological function was evaluated using a 0-4-point neurological score: 0=no neurological dysfunction; 1=failure to extend left forelimb fully when lifted by tail: 2=circling to the contralateral side; 3=falling to the left; 4=no spontaneous walk or in a comatose state, or barrel rolling. All scores were performed while being blinded to the study groups. The MST1/2 protein kinases inhibitor X1 demonstrated the capability of reducing the damages to neurological functions in the cerebral ischemia model mice, in comparison with vehicle treatment (FIG. 14B).

Accordingly, the inhibitors of MST1/2 protein kinases of the present invention are effective in preventing or treating ischemic stroke by reducing the infarct size and preserving neurological functions.

### C. Inflammatory Diseases

The applications of the inhibitors of MST1/2 protein kinases in preventing or treating inflammatory diseases were verified by inhibiting expression of cell adhesion molecules and/or inflammatory cytokines.

Arterial recruitment of inflammatory cells from the circulation and their transendothelial migration are key events in the early phase of cardiovascular and inflammatory diseases such as atherosclerosis, restenosis, heart failure and ischemic stroke. In response to several inflammatory stimuli, such as tumor necrosis factor (TNF)-α and interleukin-1β (IL)-1β, endothelial cells (ECs) undergo inflammatory activation, resulting in an increased surface expression of cell adhesion molecules, such as intercellular adhesion molecule (ICAM)-1, vascular cell adhesion molecule (VCAM)-1, and E-selectin, which contributes to recruitment of inflammatory cells to arterial wall and their transmigration across the arterial wall. Genetic deficiencies of adhesion molecules in mice are associated with decreased atherosclerosis. Therefore, modulation of the expression of adhesion molecules on ECs is an important indicator for the prevention and treatment of inflammatory diseases such as atherosclerosis.

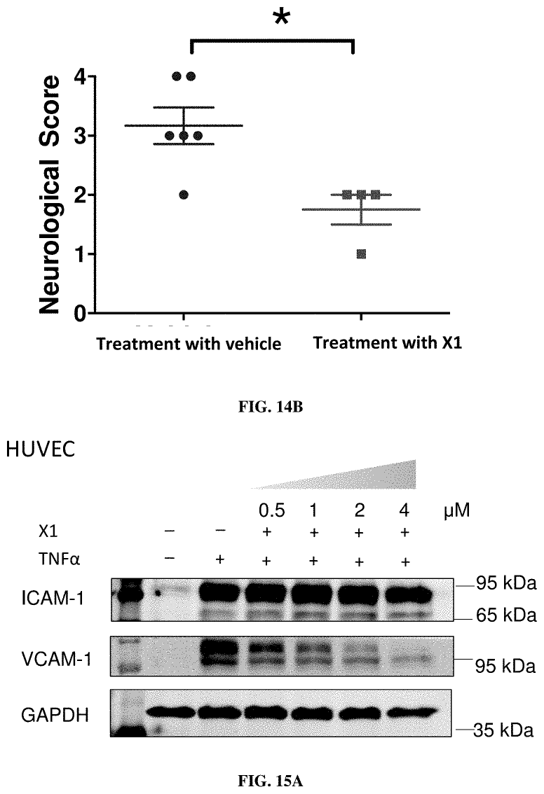

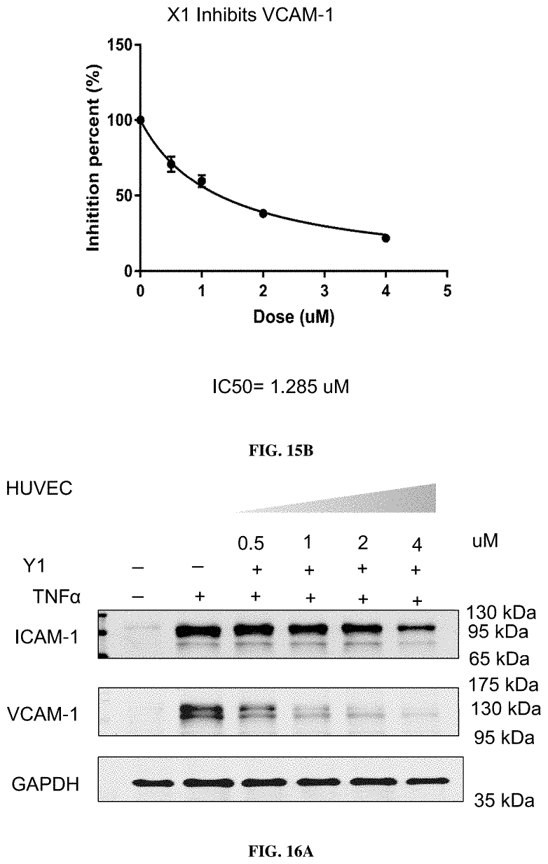

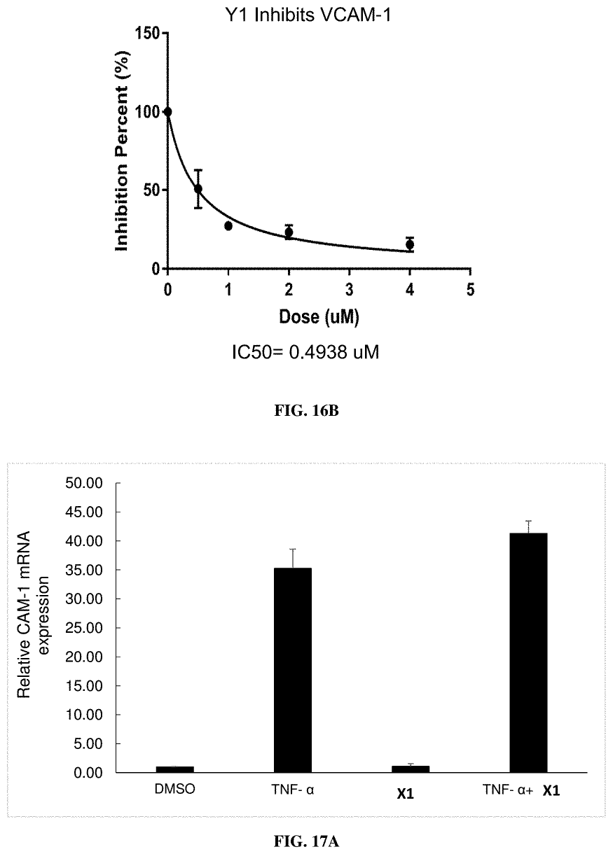

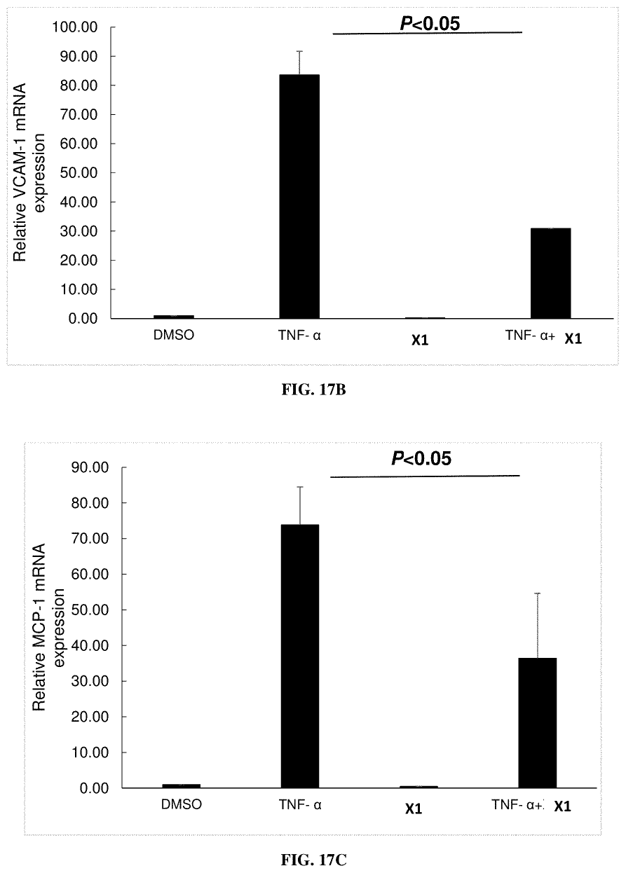

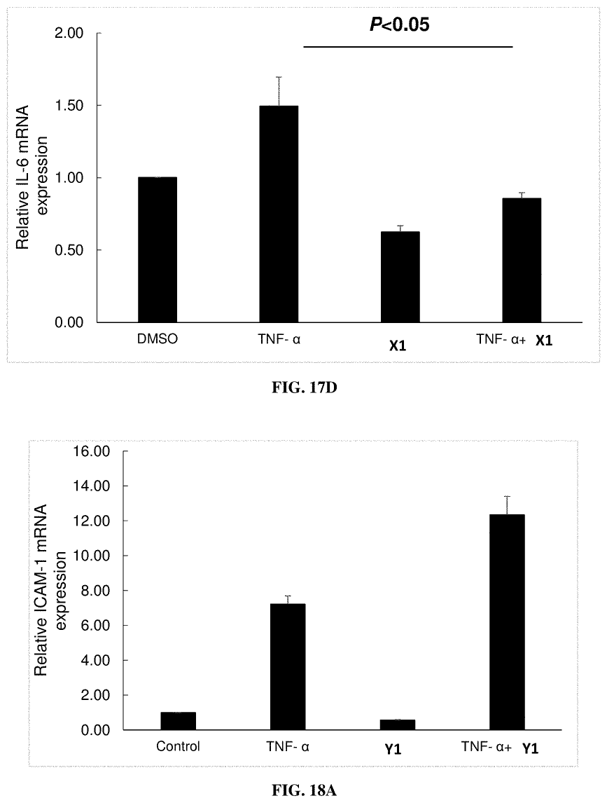

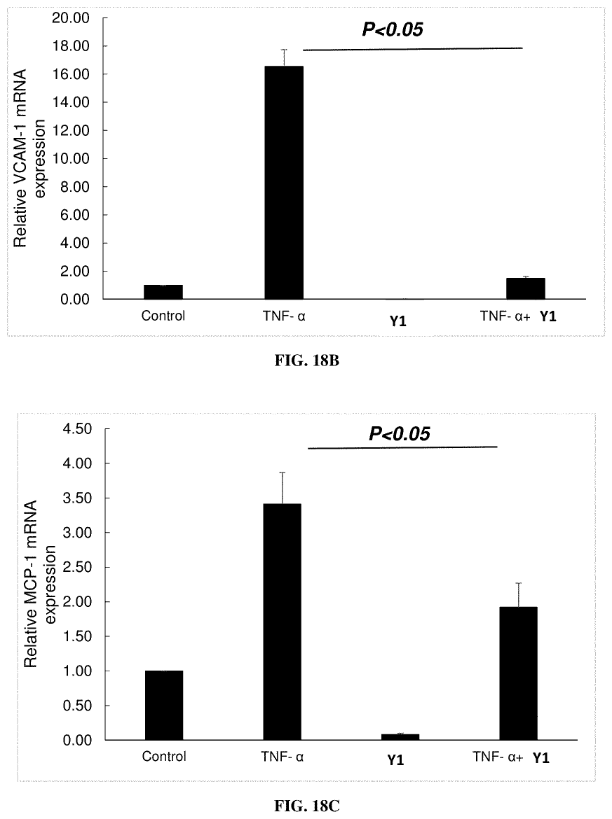

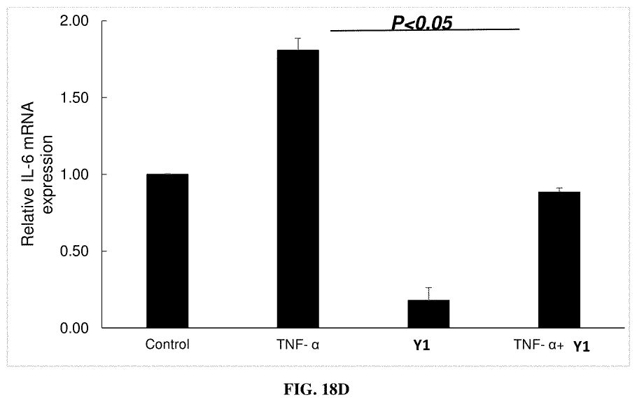

The effect of inhibitors of MST1/2 protein kinases (X1 and Y1) on the expression of cell adhesion molecules in vascular endothelial cells was measured. First, human umbilical vascular ECs (HUVECs) were pretreated with 1 μM MST1/2 inhibitors (X1 and Y1) for 1 hour and then stimulated with TNF-α (20 ng/mL) and IL-1β (10 ng/mL) for 12 hours. The expression of VCAM-1 and ICAM-1 was detected by western blot. The expression of cell adhesion molecules was reduced by the inhibitors of MST1/2 protein kinases. For example, as shown in FIG. 15A, X1 at the concentration of 1 μM substantially inhibited VCAM-1 expression, while the expression of ICAM-1 was barely affected. In addition, X1 inhibits VCAM-1 expression in a dose dependent manner with an IC50 of 1.29 μM (FIG. 15B). Likewise, Y1 had a similar inhibitor effect on the expression of VCAM-1 in HUVECs, with an IC50 of 0.49 μM (FIGS. 16A-16B). Furthermore, both compounds X1 and Y1 markedly inhibited the mRNA levels of inflammatory cytokines in HUVEC, such as MCP-1 and IL-6, in response to TNFα stimulation, as determined by qPCR (FIGS. 17A-17D and 18A-18D).

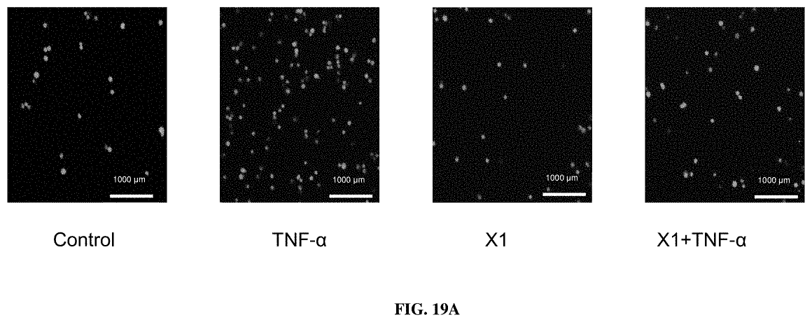

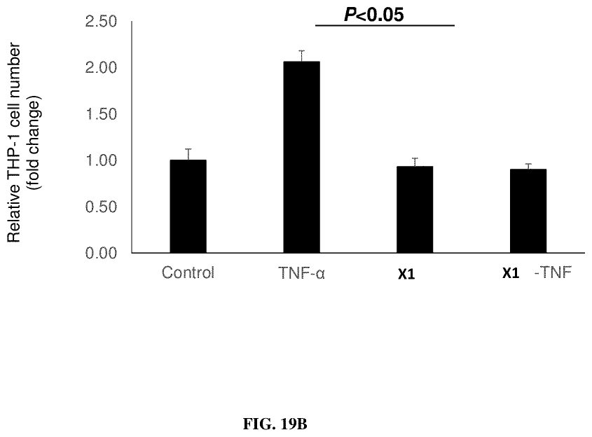

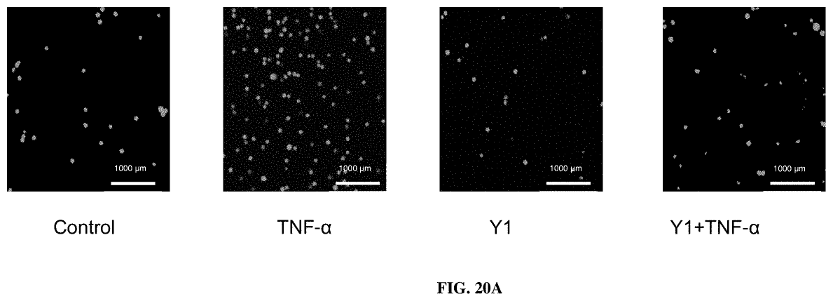

To substantiate the functional significance of inhibitors of MST1/2 protein kinases in activating endothelial cells, the monocyte adhesion to the activated endothelial cells was further examined. As shown in FIGS. 19A-19B and 20A-20B, the adhesion of THP-1 cells to TNFα-activated ECs was markedly inhibited by both X1 and Y1, indicating that inhibitors of MST1/2 protein kinases exerted potent anti-inflammatory effects in ECs.

Cell adhesion was assessed as the follows: THP1 cells were labeled with calcein-AM (Invitrogen) according to the instructions of the manufacturer. After the HUVECs were stimulated and washed, 2.5×105 calcein-labeled THP1 cells were added to each well and allowed to interact with MST1/2 inhibitors for 60 minutes at 370° C. Unbound cells were removed by gently washing with complete medium, and the number of attached cells was counted on an inverted fluorescent microscope.

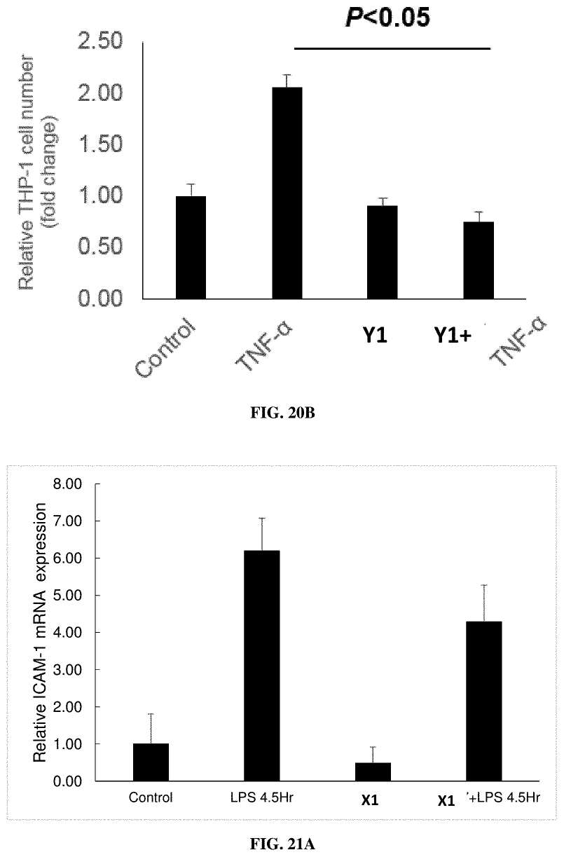

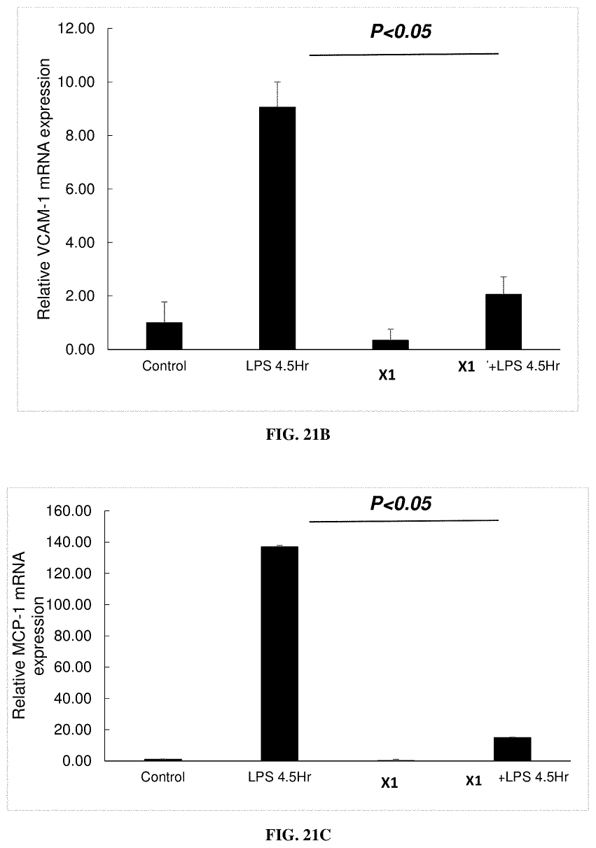

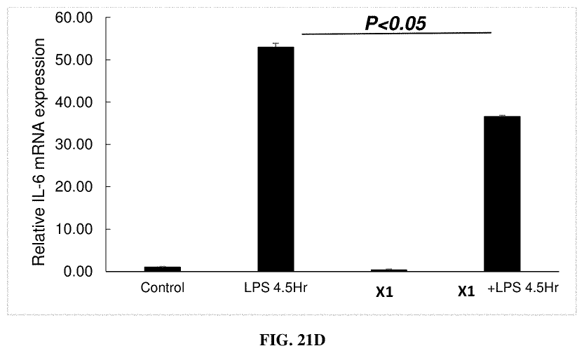

The therapeutic capacity of inhibitors of MST1/2 protein kinases for inflammatory diseases was also demonstrated by a murine septic model by injecting mice with LPS (2.5 mg/kg, i.p.). In this regard, the mice were pretreated with X1 twice at 24 hrs and 3 hrs, respectively, prior to the injection of lipopolysaccharides (LPS). 6 hrs after injection of LPS to induce the sepsis model, the expression of inflammatory cytokines in the mouse lung was determined by real-time PCR. As shown in FIGS. 21A-21D, X1 markedly suppressed the mRNA levels of ICAM-1, VCAM-1, MCP-1 and IL-6, indicating that X1 has an in vivo anti-inflammatory effect.

Accordingly, the inhibitors of MST1/2 protein kinases of the present invention are effective in preventing or treating inflammatory diseases, such as sepsis.

### Other Diseases and Disorders

In addition to these exemplary diseases and disorders described above, utility of the inhibitors of MST1/2 protein kinases in preventing or treating other diseases and disorders may be determined or confirmed by various assays. For example, the tissue protective activity of the MST1/2 inhibitors may be confirmed using various assays known in the art and disclosed within U.S. Patent Publication Nos: US 2009/0136519A1, US 2004/0122216A1, and US 2009/0221482A1. Additionally, one of ordinary skill in the art will recognize that the inhibitors of MST1/2 protein kinases' ability to prevent, mitigate or treat a disease or disorder associated with tissue damage or damage, effects or symptoms resulting therefrom may be confirmed through various assays both in vitro and in vivo, although in certain embodiments in vivo assays may be preferred.

### D. Tissue Protective Assays and Models

The inhibitors of MST1/2 protein kinases of the present invention exhibit tissue protective properties, i.e. anti-apoptotic, neuritogenic, neuroprotective, anti-cachectic, anti-inflammatory etc. These inhibitors may be tested for tissue protective activity, e.g., protecting cells, tissues or organs. Protective activities may be further tested using in vitro and in vivo assays. In vitro tests that are indicative of tissue protective activity include, for example, cell proliferation assays, cell differentiation assays, or detecting the presence of proteins or nucleic acids upregulated by tissue protective receptor complex, e.g. tissue protective cytokine receptor complex, activity, e.g., nucleolin, neuroglobin, cytoglobin, or frataxin. Neuroglobin, for example, may be involved in facilitating the transport or the short-term storage of oxygen. Therefore, oxygen transport or storage assays may be used as an assay to identify or screen for compounds which modulate tissue protective activity.

Neuroglobin is expressed in cells and tissues of the central nervous system in response to hypoxia or ischemia and may provide protection from injury (Sun et al. PNAS, 98:15306-15311, 2001; Schmid et al., J. Biol. Chem., 276:1932-1935, 2003, each of which is incorporated by reference herein in its entirety). Cytoglobin may play a similar role in protection of tissues or organs, but is expressed in a variety of tissues at varying levels (Pesce et al., EMBO, 3:1146-1151, 2002, which is incorporated by reference herein in its entirety). In one embodiment of the invention, the levels of an upregulated protein in a cell may be measured before and after contacting the MST1/2 inhibitors to a cell. In certain embodiments, the presence of an upregulated protein associated with tissue protective activity in a cell, may be used to confirm the tissue protective activities of the MST1/2 inhibitors.

Nucleolin may protect cells from certain damage. It plays numerous roles in cells including modulation of transcription processes, sequence specific RNA-binding protein, cytokinesis, nucleogensis, signal transduction, apoptosis induced by T-cells, chromatin remodelling, or replication. It can also function as a cell surface receptor DNA/RNA helicase, DNA-dependent ATPase, protein shuttle, transcription factor component, or transcriptional repressor (Srivastava and Pollard, FASEB J., 13:1911-1922, 1999; and Ginisty et al., J. Cell Sci., 112:761-772, 1999, each of which is incorporated by reference herein in its entirety).

Frataxin is a protein involved with mitochondrial iron metabolism and has previously been shown to be strongly up-regulated by EPO both in vivo and in vitro (Sturm et al., Eur J Clin Invest 35: 711, 2005, which is incorporated by reference herein in its entirety).

Expression of a protein may be detected by detecting mRNA levels corresponding to the protein in a cell. The mRNA can be hybridized to a probe that specifically binds a nucleic acid encoding the upregulated protein. Hybridization may consist of, for example, Northern blot, Southern blot, array hybridization, affinity chromatography, or in situ hybridization. The mRNA expression levels can be quantitated with new generations of RNA-seq techniques.

Tissue protective activity of the inhibitors of MST1/2 protein kinases can also be detected using in vitro neuroprotection assays. For example, primary neuronal cultures may be prepared from new born rat hippocampi by trypsinization, and cultured as by any method known in the art and/or described herein e.g. in MEM-II growth medium (Invitrogen), 20 mM D-glucose, 2 mM L-glutamine, 10% Nu-serum (bovine; Becton Dickinson, Franklin Lakes, N.J.), 2% B27 supplement (Invitrogen), 26.2 mM NaHCO₃, 100 U/ml penicillin, and 1 mg/ml streptavidin (see, e.g., Leist et al., Science 305:239-242, 2004, hereby incorporated by reference in its entirety). One day after seeding, 1 μM cytosinearabino-furanoside is added. 13 day old cultures are then preincubated with increasing doses of the MST1/2 inhibitors of interest (3-3000 μM) for 24 h. On day 14, the medium is removed and the cultures challenged with 300 μM NMDA in PBS at room temperature (RT). After 5 min, pre-conditioned medium is returned to the cultures which are then returned to the incubator for 24 h. The cells are fixed in paraformaldehyde, stained by Hoechst 33342 (Molecular Probes, Eugene, Oreg.) and condensed apoptotic nuclei may be counted. NGF (50 ng/ml) and MK801 (1 μM) are included as positive controls.

Animal model systems can be used to demonstrate the tissue protective activity of the inhibitors of MST1/2 protein kinases to demonstrate their safety and efficacy for different types of tissue damage, disease, condition, or syndrome of interest. Animal models for various diseases and disorders are known in the art. For example, protection against the onset of acute experimental allergic encephalomyelitis in Lewis rats, restoration or protection from diminished cognitive function in mice after receiving brain trauma, cerebral ischemia (“stroke”) or seizures stimulated by excitotoxins (Brines et al., PNAS, 97:10295-10672, 2000, which is incorporated by reference herein in its entirety), protection from induced retinal ischemia (Rosenbaum et al., Vis. Res. 37:3443-51, 1997, which is incorporated by reference herein in its entirety), protection from injury to the sciatic nerve, and protection from ischemia-reperfusion injury to the heart (in vitro cardiomyocyte studies and in vivo ischemia-reperfusion injury, see, e.g., Calvillo et al., PNAS 100:4802-4806, 2003, and Fiordaliso et al., PNAS 102:2046-2051, 2005, each of which is hereby incorporated by reference in its entirety). Animal models related to spinal cord injury, ischemic stroke, peripheral nerve damage, wounds, or damage to the heart, eyes, kidneys, etc. are also known in the art. Such assays are described in further detail in Grasso et al. Med Sci Monit 10: BR1-3, 2004, PCT publication Nos. WO 2002/053580 and WO 2007/019545, each of which is incorporated by reference herein in its entirety.

### E. Assays for Specific Indications

Specific indications for the inhibitors of MST1/2 protein kinases may be verified by various assays. Two exemplary indications are provided for illustrative purposes. One is preventing or treating tissue damages induced by a toxic agent. Another is preventing or treating tissue damages associated with inflammation.

A variety of assays known in the art may be used to determine the inhibitors of MST1/2 protein kinases' ability to prevent, treat, ameliorate, or manage damage, effects or symptoms resulting from exposure to a toxic agent. In general, this is accomplished by selecting an appropriate cell line, subjecting that cell to a toxic agent of interest and treating a portion of the cells with an inhibitor of MST1/2 protein kinases and determining the cells survival or response in the presence of the toxic agent and the inhibitor of MST1/2 protein kinases of interest. If the cell exhibits improved survival or a reduction of damage, effects or symptoms in the presence of the inhibitor of MST1/2 protein kinases, the inhibitor of MST1/2 protein kinases can be considered to be a possible therapeutic for toxic exposure. Further one of ordinary skill in the art will recognize that the inhibitors of MST1/2 protein kinases' protective ability can be evaluated by treating the cells with the inhibitors of MST1/2 protein kinases prior to the toxic agent challenge.

Exemplary cell lines for testing protective ability against tissue damages induced by a chemical agent include: a) skin cell lines such as J-774 (mouse macrophage derived cell line), CHO-K1 (strain of epithelial cell line derived from Chinese hamster ovary cells), and HeLa (human cervical carcinoma) (Sawyer, et al., Eplasty, 8:e25, 2008); b) corneal cell lines for vesicant agents (Amir, et al., Proceedings of the U.S. Army Medical Defense Bioscience Review, Aberdeen Proving Ground, MD (2004)); c) macrophages (Amir, et al., J Appl Toxicol, 20 Suppl 1:S51-8, 2000); d) upper respiratory tract cell lines (Andrew and Lindsay, Hum Exp Toxicol 17(7):387-95, 1998; Calvet et al., Hum Exp Toxicol 18(2):77-81, 1999; Langford, et al., Hum Exp Toxicol 15(8):619-24, 1996); e) skin models (Blaha et al., J Appl Toxicol 20 Suppl 1:S101-8, 2000) Exemplary cell lines for testing protective ability against tissue damages induced by a radiation agent include: a) endothelial cells (Abderrahmani, et al., Radioprotection 2008, vol 43, no. 5, 2008), b) neuroimmune cells (afferent nerves, enteric sensory nerves, mast cells) (Wang, et al., British Journal of Radiology, 80:S41-S48, 2007), c) blood or lymphocyte cultures (Lloyd, et al., Phys Med Biol 18(3):421-31, 1973; Lloyd, et al., Mutat. Res. 179(2):197-208, 1987; Blakely et al., Stem Cells 13 (Suppl 1):223-30, 1995; Gotoh et al., Int. J. Radiation. Biol. 81(1):33-40, 2005).

Further, suitable in vivo assays are known in the art for evaluating the effect of tissue protection after toxic agent exposure. Animal models using rats, mice, guinea pigs, rabbits, pigs, sheep, ferrets, dogs and non-human primates are contemplated as well as transgenic animals that are particularly susceptible to a toxic agent (CD46 mice). In particular, exemplary animal models for testing protective ability against tissue damages induced by chemical agents include: (1) Reid, Sulfur mustard induced skin burns in weanling swine evaluated clinically and histopathologically, Journal of applied toxicology, 20(S1):5153-5160, 2001; (2) Isidore, et al., A dorsal model for cutaneous vesicant injury 2-chloroethyl ethyl sulfide using c57b1/6 mice, Cutaneous and ocular toxicology, 26(3):265-276, 2007; (3) Kassa, et al., The Choice: HI-6, pradoxime or Obidoxime against Nerve Agents?, www.asanite.com/ASANews-97/Antidot-Choice.html, (4) Shih, et al., Organophosphorus nerve agents-induced seizures and efficacy of atropine sulfate as anticonvulsant treatment, Pharmacol-Biochem-Behav. 64(1):147-53, 1999, (5) Luo, et al., Comparison of oxime reactivation and aging of the nerve agent-inhibited monkey and human acetylcholinesterases, Chemico-Biological Interactions, 175(1-3):261-266, 2008.

Exemplary animal models for testing protective ability against tissue damages induced by radiation agents include: (1) Blakely et al., In Vitro and Animal Models of Partial-Body Dose Exposure: Use of Cytogenic and Molecular Biomarkers for Assessment of Inhomogeneous Dose Exposures and Radiation Injury, PB-Rad-Injury 2008 Workshop, May 5-6, 2008 AFRRI, Bethesda, Md.; (2) Augustine, et al., Meeting Report: Animal Models of Radiation Injury, Protection and Therapy, Radiation Research 164:100-109, 2005; (3) Houchen, et al. Prosurvival and antiapoptotic effects of PGE₂ in radiation injury are mediated by EP₂ receptor in intestine, Am J Physiol Gastrointest Liver Physiol, 284:G490-G498, 2003; (4) Chen, Animal Models for Acquired Bone Marrow Failure Syndromes, Clinical Medicine & Research 3(2):102-108, 2005.

Additionally, various in vitro models of inflammation may be used to evaluate inhibitors of MST1/2 protein kinases' ability to protect or treat the damage, symptoms, or effects of inflammation on the body. Initially, the ability of the inhibitors of MST1/2 protein kinases to modulate an inflammatory mediator (e.g., ICAM and VCAM) can be confirmed by measuring the levels of the inflammatory mediator in an inflammatory assay after treatment with the inhibitors of MST1/2 protein kinases, including but not limited to, ELISA, cytometric bead array analysis, high-sensitivity and immunonephelometric assays. For example, to determine if the inhibitors of MST1/2 protein kinases modulate either TNFα or IL-1, a murine model of LPS-mediated cytokine production would be performed. Some mice in the murine model would be pretreated with the inhibitors of MST1/2 protein kinases and then challenged with LPS while others would be saline treated. Blood would then be collected and the TNFα and IL-1 levels in the blood could be determined by an ELISA kit (OPT-EIA mouse TNFα and IL-1 ELISA kits from BD Biosciences). If the TNFα levels in the treated animals are lower than the TNFα levels in the saline treated animals then the inhibitors of MST1/2 protein kinases could be considered to modulate TNFα. Preferably, the inhibitors of MST1/2 protein kinases would be tested for its ability to modulate more than one inflammatory mediator, and more preferably it would be a mediator other than or in addition to TNFα, and most preferably it would be histamine. Similarly, the inhibitors of MST1/2 protein kinases may be tested in additional in vitro assays including, but not limited to, those disclosed in Lopata, Current Allergy & Clinical Immunology, 19:18-20, 2006, (histamine and tryptase assays), and Arulmozhi et al., Indian Journal of Pharmacology, 37:96-102, 2005, (5-lipoxygenase (5-LO), cyclo-oxygenase (COX), Leukotrine B4 (LTB4) and nitric oxide synthase (NOS)).

Further, in vivo assays of inflammation may be useful in evaluating the inhibitors of MST1/2 protein kinases' utility for preventing or treating inflammation. In vivo assays include, but not limited to, murine EAE models, those utilizing transgenic mice such as MDBiosciences DSS IBD murine model of severe colitis, the MDBioscience TNBS IBD murine model of inflammatory bowel disease, models involving IL-1 knockout mice disclosed within U.S. Pat. No. 6,437,216, or models of transgenic mice involving TNFα as disclosed within Probert et al., PNAS, 92:11294-11298, 1995, Kontoyiannis et al., Immunity 10:387-398, 1999, Keffer et al., EMBO J. 10(13):4025-31, 1991, or models using chemical or synthetic challenges to induce the inflammation such as models of asthma and chronic obstructive pulmonary disease disclosed in JPET 307:373-385, 2003, adjuvant arthritis models as disclosed in EP 1 777 234; murine LPS shock models, murine LPS lung models, acute paw inflammation models, or histidine challenge wheal formation model as known in the art.

Further, the efficacy of the inhibitors of MST1/2 protein kinases in humans may be verified by using well-known clinical studies such as the skin prick test and bronchoprovocation test disclosed in Ravensberg et al., Clinical and Experimental Allergy, 37:100-107, 2007; asthma studies as disclosed in Diamant et al., Respiratory Medicine, 102:332-338, 2008, or nasal allergen challenge as disclosed in Boot et al. Allergy, 62:378-384, 2007.

In the present application, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. All citations (e.g., scientific journal publications, patents, and other reference material) mentioned herein are hereby incorporated herein by reference to the same extent as if each individual citation was specifically and individually indicated to be incorporated by reference.

1. A method of preventing or treating a disease or disorder associated with tissue damage using a MST1/2 protein kinase inhibitor, said method comprising a step of:
administering the MST1/2 protein kinase inhibitor to a subject having the disease or disorder,
wherein the MST1/2 protein kinase inhibitor is administered at a dose of from about 0.1 mg/kg to about 100 mg/kg based on the bodyweight of the subject and at a frequency of once in a period of from 6 hours to 20 days, and
the MST1/2 protein kinase inhibitor has formulas I, II, III, IV: 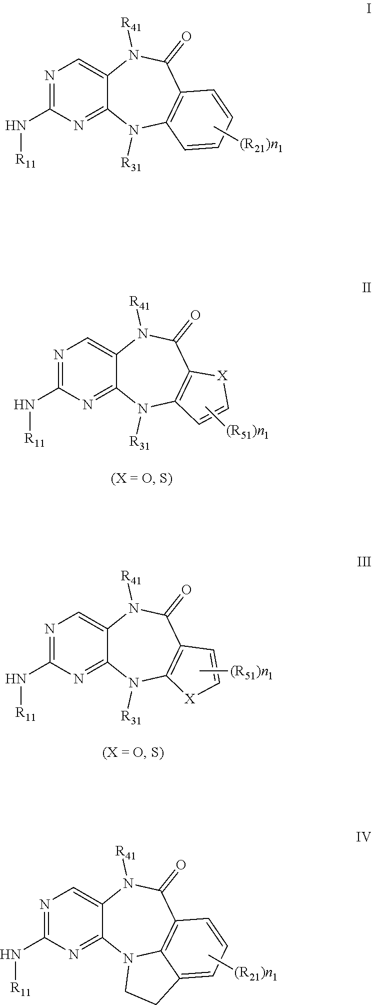
wherein: n1 is selected from 0, 1, 2, 3 or 4;
R₁₁ is selected from:
1) C1-C6 alkyl, optionally substituted with halogen, amino, nitro, cyano; C1-C6 alkyl containing oxygen; C3-C7 cycloalkyl, which is optionally substituted with halogen, amino, nitro, cyano; C6-C10 aryl, optionally substituted by halogen, nitro, amino, hydroxy, cyano; C3-C6 alkenyl;
2) 2-N, N-dimethylaminoethyl, 2-hydroxyethyl, 2-N, N-diethylaminoethyl, 2-N, N-diisopropylamino ethyl, 2-morpholinyl ethyl, 2-thiomorpholinyl ethyl, 2-(4-N-piperazinyl-methyl) ethyl, 3-N, N-dimethylaminopropyl, 3-N, N-diethylaminopropyl, 3-N, N-diisopropyl-aminopropyl, 3-morpholinyl propyl, 3-thiomorpholinyl propyl, 3-(4-N-methylpiperidinyl) propyl, 4-N, N-dimethylamino-cyclohexyl, 4-N, N-diethylamino cyclohexyl, N-methyl-4-piperidinyl, N-ethyl-4-piperidinyl, N-isopropyl-4-piperidinyl, 1,3-dimethyl-5-pyrazolyl, 1-methyl-4-pyrazolyl, 3-methyl-5-isoxazolinyl, 1-(N-methyl-4-piperidinyl)-4-pyrazolyl, 1-(N-tert-butoxyl formyl-4-piperidinyl)-4-pyrazolyl; 3) 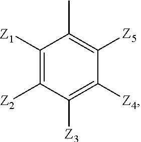
wherein Z₁, Z₂, Z₃, Z₄, Z₅ are each independently selected from:
(1) hydrogen, halogen, nitro, amino, hydroxy, cyano,
(2) C1-C6 alkyl, C1-C6 alkoxy, C1-C6 alkyl containing oxygen, C1-C6 alkyl containing fluorine, C1-C6 alkoxy containing fluorine, 4-piperidinyl, N-methyl yl-4-piperidinyl,
(3) N, N-dimethylamino, N, N-diethylamino, N, N-diisopropylamino, 2-N, N-dimethylaminoethylamino, 2-morpholino ethylamino, 2-ethylamino thiomorpholinyl, 2-(4-N-methylpiperazinyl) ethylamino, 3-N, N-dimethyl-aminopropyl amino, 3-N, N-diethylaminopropyl-amino, 3-N, N-diisopropylamino propylamino, 3-morpholin-propylamino, 3-thiomorpholinyl propylamino, 3-(4 N-methylpiperazinyl) propylamino, N-methylpiperidinyl-4-amino, N-ethylpiperidinyl-4-amino, N-isopropyl-piperidinyl-4-amino,
(4) 2-N, N-dimethylaminoethoxyl, 2-N, N-diethyl-aminoethoxy, 2-N, N-diisopropyl-aminoethoxyl, 2-(N-methylpiperazinyl) ethoxyl, 2-(N-acetyl-piperazinyl) ethoxyl, 2-morpholino-ethoxyl, 2-thiomorpholino-ethoxyl, 2-piperidinyl ethoxyl, 3-N, N-dimethylamino-propoxyl, 3-N, N-diethylamino-propoxyl, 3-N, N-diisopropylamino propoxyl, 3-(N-methylpiperazinyl) propoxyl, 3-(N-acetyl-piperazinyl) propoxyl, 3-morpholinyl-propoxyl, 3-thiomorpholinyl propoxyl, 3-piperidinyl-propoxyl, 2-pyridyl-methoxyl, 3-pyridyl-methoxyl, 4-pyridyl methoxyl, phenylmethoxyl, monohalogen-substituted phenylmethoxyl, homodihalogen-substituted phenylmethoxyl, heterodihalogen-substituted phenylmethoxyl,
(5) piperidinyl, 4-N, N-dimethylamino-piperidinyl, 4-N, N-diethylamino-piperidinyl, 4-N, N-diisopropylamino piperidinyl, 4-hydroxy piperidinyl, morpholinyl, 3,5-dimethyl morpholinyl, thiomorpholinyl, tetrahydropyrrolyl, 3-N, N-dimethyl-tetrahydropyrrolyl, 3-N, N-diethyl-tetrahydropyrrolyl, N-methyl-piperazinyl, N-ethyl-piperazinyl, N-isopropyl-piperazinyl, N-acetyl-piperazinyl, N-tert-butoxyl formyl piperazinyl, N-methylsulfonyl-piperazinyl, N-(2-hydroxylethyl) piperazinyl, N-(2-cyanoethyl) piperazinyl, N-(3-hydroxylpropyl) piperazinyl, N-(2-N, N-dimethylethyl) piperazinyl, N-(2-N, N-diethyl-ethyl) piperazinyl, N-(3-N, N-dimethylpropyl) piperazinyl, N-(3-N, N-diethyl-propyl) piperazinyl, 2-oxo-piperazinyl, 2-oxo-piperazin-4-yl, imidazolyl, 4-imidazolyl,
(6) 4-(N-methylpiperazinyl) piperidinyl, 4-(N-ethyl-piperazinyl) piperidinyl, 4-(N-isopropyl-piperazinyl) piperidinyl, 4-(N-acetyl-piperazinyl) piperidinyl, 4-(N-t-butoxyl-formyl-piperazinyl) piperidinyl, 4-(N-methylsulfonyl-piperazinyl) piperidinyl, 4-(N-(2-hydroxylethyl) piperazinyl) piperidinyl, 4-(N-(2-cyanoethyl) piperazinyl) piperidinyl, 4-(N-(3-hydroxylpropyl) piperazinyl) piperidinyl, 4-(N-(2-N, N-dimethyl-ethyl) piperazinyl) piperidinyl, 4-(N-(2-N, N-diethyl ethyl) piperazinyl) piperidinyl, 4-(N-(3-N, N-dimethyl-propyl) piperazinyl) piperidinyl, 4-(N-(3-N, N-diethyl-propyl) piperazinyl) piperidinyl, 4-(tetrahydropyrrolyl) piperidinyl, 4-(3-N, N-dimethyl-tetrahydropyrrolyl) piperidinyl, N—(N-methyl-4-piperidinyl) piperazinyl, N—(N-ethyl-4-piperidinyl) piperazinyl,
(7) hydroxy sulfonyl, aminosulfonyl, sulfonyl methylamino, ethylamino sulfonyl group, a sulfonyl group propylamino, isopropylamino-sulfonyl, aminosulfonyl cyclopropyl, cyclobutyl aminosulfonyl, cyclopentyl aminosulfonyl, piperidinyl-sulfonyl, 4-hydroxyl-piperidinyl-1-sulfonyl, 4-N, N-dimethyl-piperidinyl-1-sulfonyl, 4-N, N-diethyl-piperidinyl-1-sulfonyl, pyrrolidinyl-1-sulfonyl, 3-N, N-dimethyl-pyrrolidinyl-1-sulfonyl, 3-N, N-diethyl-pyrrolidinyl-1-sulfonyl, N-methyl-piperazinyl-sulfonyl, N-ethylpiperazinyl-1-sulfonyl, N-acetyl-piperazinyl-1-sulfonyl, N-tert-butoxylformyl-piperazinyl-1-sulfonyl, N-(2-hydroxylethyl) piperazinyl-1-sulfonyl, N-(2-cyanoethyl) piperazinyl-1-sulfonyl, N-(2-N, N-dimethyl ethyl) piperazinyl-1-sulfonyl, N-(2-N, N-diethyl-ethyl) piperazinyl-1-sulfonyl, N-(3-hydroxylpropyl) piperazinyl-1-sulfonyl, N-(3-N, N-dimethylamino-propyl) piperazinyl-1-sulfonyl, N-(3-N, N-diethylamino-propyl) piperazinyl-1-sulfonyl, morpholinyl-1-sulfonyl, 3,5-dimethyl-morpholinyl-1-sulfonyl, 4-(N-methyl-1-piperazinyl) piperidinyl-1-sulfonyl, 4-(N-ethyl-1-piperazinyl) piperidinyl-1-sulfonyl, 4-(N-acetyl-1-piperazinyl) piperidinyl-sulfonyl, N—(N-methyl-4-piperidinyl) piperazinyl-1-sulfonyl,
(8) amino formyl, methylamino formyl, ethylamino formyl, propylamino formyl, isopropylamino formyl, cyclopropylamino formyl, cyclobutylamino formyl, cyclopentylamino formyl, piperidinyl-1-formyl, 4-hydroxy-piperidinyl-1-formyl, 4-N, N-dimethyl-piperidinyl-1-formyl, 4-N, N-two ethylpiperidinyl-1-formyl, tetrahydropyrrolyl-1-formyl, 3-N, N-dimethyl-tetrahydropyrrolyl-1-formyl, 3-N, N-diethyl-tetrahydropyrrolyl-1-formyl, N-methyl-piperazinyl-1-formyl, N-ethyl-piperazinyl-1-formyl, N-acetyl-piperazinyl-1-formyl, N-tert-butoxyl-formyl-piperazinyl-1-formyl, N-(2-hydroxyethyl) piperazinyl-1-formyl, N-(2-cyanoethyl) piperazinyl-1-formyl, N-(2-N, N-dimethyl-ethyl) piperazinyl-1-formyl, N-(2-N, N-diethyl-ethyl) piperazinyl-1-formyl, N-(3-hydroxypropyl) piperazinyl-1-formyl, N-(3-N, N-dimethyl-propyl) piperazinyl-1-formyl, N-(3-N, N-diethyl propyl) piperazinyl-1-formyl, morpholinyl-1-formyl, 3,5-dimethyl-morpholinyl-1-formyl, 4-(N-methyl-1-piperazinyl) piperidinyl-1-formyl, 4-(N-ethyl-1-piperazinyl) piperidinyl-1-formyl, 4-(N-acetyl-1-piperazinyl) piperidinyl-1-formyl, N—(N-methyl-4-piperidinyl) piperazinyl-1-formyl,
(9) hydroxyl formyl, methoxyl formyl, ethoxyl formyl, propoxyl formyl, isopropoxyl formyl, n-butoxyl formyl, isobutoxy formyl, t-butoxyl formyl,
(10) amino formamido, methylamino formamido, ethylamino formamido, propylamino formamido, isopropylamino formamido, cyclopropylamino formamido, cyclobutylamino formamido, cyclopentylamino formamido, piperidinyl-1-formamido, 4-hydroxy-piperidinyl-1-formamido, 4-N, N-dimethyl-piperidinyl-1-formamido, 4-N, N-diethyl-piperidinyl-1-formamido, tetrahydropyrrolyl-1-formamido, 3-N, N-dimethyl-tetrahydropyrrolyl-1-formamido, 3-N, N-diethyl-tetrahydropyrrolyl-1-formamido, N-methyl-piperazinyl-1-formamido, N-ethyl-piperazinyl-1-formamido, N-acetyl-piperazinyl-1-formamido, N-tert-butoxyl formyl-piperazinyl-1-formamido, N-(2-hydroxyethyl) piperazinyl-1-formamido, N-(2-cyanoethyl) piperazinyl-1-formamido, N-(2-N, N-dimethyl-ethyl) piperazinyl-1-formamido, N-(2-N, N-diethyl-ethyl) piperazinyl-1-formamido, N-(3-hydroxypropyl) piperazinyl-1-formamido, N-(3-N, N-dimethyl-propyl) piperazinyl-1-formamido, N-(3-N, N-diethyl-aminopropyl) piperazinyl-1-formamido, morpholinyl-1-formamido, 3,5-dimethyl-morpholinyl-1-formamido, 4-(N-methyl-1-piperazinyl) piperidinyl-1-formamido, 4-(N-ethyl-1-piperazinyl) piperidinyl-1-formamido, 4-(N-acetyl-1-piperazinyl) piperidinyl-1-formamido, N—(N-methyl-4-piperidinyl) piperazinyl-1-formamido; or
(11) amino acetamido, N-tert-butoxyl formyl acetamido, N-acetylamino acetamido, acrylamido, cyclopropylamido, chloroacetamido, bromoacetamido, piperidinyl acetamido, 4-hydroxy piperidinyl acetamido, 4-N, N-dimethyl-piperidinyl-acetamido, 4-N, N-diethyl-piperidinyl acetamido, tetrahydropyrrolyl acetamido, 3-N, N-dimethyl-tetrahydropyrrolyl acetamido, 3-N, N-diethyl-tetrahydropyrrolyl-acetamido, N-methyl-piperazinyl acetamido, N-ethyl piperazinyl-acetamido, N-acetyl-piperazinyl acetamido, N-tert-butoxy formyl-piperazinyl acetamido, N-(2-hydroxyethyl) piperazinyl acetamido, N-(2-cyanoethyl) piperazinyl acetamido, N-(2-N, N-dimethylethyl) piperazinyl acetamido, N-(2-N, N-diethyl-ethyl) piperazinyl acetamido, N-(3-hydroxylpropyl) piperazinyl acetamido, N-(3-N, N-dimethyl-propyl) piperazinyl acetamido, N-(3-N, N-diethyl-propyl) piperazinyl acetamido, morpholinyl acetamido, 3,5-dimethyl-morpholinyl-acetamido, 4-(N-methyl-1-piperazinyl) piperidinyl acetamido, 4-(N-ethyl-1-piperazinyl) piperidinyl acetamido, 4-(N-acetyl-1-piperazinyl) piperidinyl acetamido, N—(N-methyl-4-piperidinyl) piperazinyl acetamido, 4-(tetrahydropyrrolyl) piperidinyl acetamido; 2-methylamino acetamido, 2-(1-methylethyl) amino acetamido; N-benzyloxy-formyl-2-methylamino-acetamido;
(12) Z₂ and Z₃ may form a substituted or unsubstituted oxygen-containing five- or six-membered ring;
the substituents may be selected from the same substituents of Z₁,
(13) Z₂ and Z₃ may form a substituted or unsubstituted nitrogen-containing five- or six-membered ring; the substituents may be selected from the same substituents of Z₁, 4) 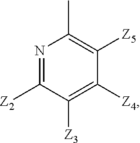
wherein Z₂, Z₃, Z₄, Z₅ are the same as the definition 3) above;
5) 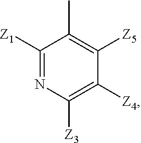
wherein Z₁, Z₃, Z₄, Z₅ are the same as the definition 3) above;
R₁ is selected from:
1) a hydrogen, halo, nitro, amino, cyano;
2) C1-C6 alkyl, optionally substituted by halogen, nitro, amino, cyano; —O—C1-C6 alkyl, optionally substituted by halogen, nitro, amino, cyano; a C1-C6 oxygen-containing alkyl;
3) methylthio, ethylthio, isopropylthio, methylsulfinyl, ethylsulfinyl, propyl sulfinyl, methylsulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
R₃₁ is selected from:
Hydrogen; C1-C6 alkyl, optionally substituted by halogen, nitro, amino, cyano; C3-C7 cycloalkyl, which is optionally substituted by halogen, nitro, amino, cyano;
R₄₁ is selected from:
hydrogen; C1-C6 alkyl, optionally substituted by halogen, nitro, amino, cyano; C3-C7 cycloalkyl, which is optionally substituted by halogen, nitro, amino, cyano;
R₅₁ is selected from:
1) a hydrogen, halo, nitro, amino, cyano;
2) C1-C6 alkyl, optionally substituted by halogen, nitro, amino, cyano; —O—C1-C6 alkyl, optionally substituted by halogen, nitro, amino, cyano; a C1-C6 oxygen-containing alkyl;
3) methylthio, ethylthio, isopropylthio, methylsulfinyl, ethyl sulfinyl, propyl sulfinyl, methylsulfonyl, ethylsulfonyl, isopropylsulfonyl, amino sulfonyl, ethylamino sulfonyl, propylamino sulfonyl, isopropylamino-sulfonyl, cyclopropylamino sulfonyl, hydroxyl formyl, methoxyl formyl, ethoxyl formyl, propoxyl formyl, isopropoxyl formyl, n-butoxyl formyl, isobutoxyl formyl, t-butoxyl formyl, amino formyl, methylamino formyl, ethylamino formyl, propylamino formyl, isopropylamino formyl, cyclopropylamino formyl, cyclobutylamino formyl, cyclopentylamino formyl, acetamido, propionamido, n-butyl amido, isobutyl amido, cyclopropyl formamido, cyclobutyl formamido, cyclopentyl formamido, methylsulfonamido, ethylsulfonamido, propylsulfonamido, isopropylsulfonamido, dimethyl phosphinyl, diethyl phosphinyl, diisopropyl phosphinyl;
or a stereoisomer of the above compounds, a prodrug thereof, a pharmaceutically acceptable salt thereof, or a pharmaceutically acceptable solvate thereof.

2. The method of claim 1, wherein the disease or disorder associated with tissue damage is a trauma to brain, a trauma to spinal cord, a trauma to peripheral nerves, a trauma to retinal or a trauma to heart.

3. The method of claim 2, wherein the trauma to brain is ischemic stroke, blunt trauma, or subarachnoid hemorrhage.

4. The method of claim 2, wherein the trauma to spinal cord is spinal cord ischemia or spinal cord blunt force trauma.

5. (canceled)

6. The method of claim 2, wherein the trauma to retinal is macular edema, diabetic retinopathy, or glaucoma.

7. The method of claim 2, wherein the trauma to heart is myocardial infarct, or chronic heart failure.

8. The method of claim 1, wherein the disease or disorder associated with tissue damage is an organ failure.

9. The method of claim 8, wherein the organ failure is selected from diabetes mellitus type I or II, nephrosis, fatty liver diseases, failure of gonads, failure of pancreas, failure of kidney, failure of heart, failure of lung, failure of liver, and failure of bowel.

10. The method of claim 1, wherein the disease or disorder associated with tissue damage is a disease or disorder caused by exposure to a toxic agent.

11. The method of claim 10, wherein the toxic agent is selected from chemotherapeutic agents, chemical agents and radiation agents.

12. The method of claim 1, wherein the disease or disorder associated with tissue damage is an inflammatory disease.

13. The method of claim 12, wherein the inflammatory disease is selected from sepsis, inflammatory bowel diseases, Crohn's disease, ulcerative colitis, ileitis, enteritis, and acute nephritis.

14. The method of claim 1, wherein the disease or disorder associated with tissue damage is a degenerative disease.

15. The method of claim 14, wherein the degenerative disease is selected from muscular dystrophies, myotonic dystrophy, and neurodegenerative diseases.

16. The method of claim 1, wherein the MST1/2 protein kinase inhibitor is administered prior to onset of the disease or disorder associated with tissue damage, during development of the disease or disorder associated with tissue damage, and/or after the disease or disorder associated with tissue damage has developed.

17. The method of claim 1, wherein the MST1/2 protein kinase inhibitor is administered from one minute to about 24 hours prior to onset of the disease or disorder associated with tissue damage.

18. The method of claim 1, wherein the MST1/2 protein kinase inhibitor is administered at a dose of from about 1 to about 10 mg/kg bodyweight.

19-20. (canceled)

21. The method of claim 1, wherein the MST1/2 protein kinase inhibitor is administered at a frequency of once in a period of from 8 hours to 10 days.

22-28. (canceled)

29. The method of claim 1, wherein the MST1/2 protein kinase inhibitor is selected from:

| | |
|---|---|
| 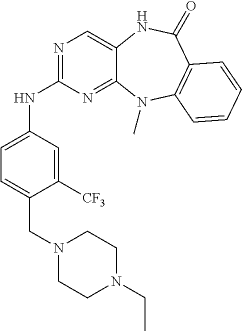 [SMILES: CCN1CCN(Cc2ccc(Nc3ncc4c(n3)N(C)c3ccccc3C(=O)N4)cc2C(F)(F)F)CC1] | IA-1 |
| (TFA Salt) | |
| 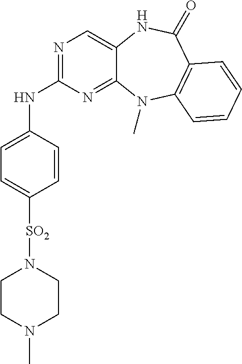 [SMILES: CN1CCN(S(=O)(=O)c2ccc(Nc3ncc4c(n3)N(C)c3ccccc3C(=O)N4)cc2)CC1] | IA-2 |
| 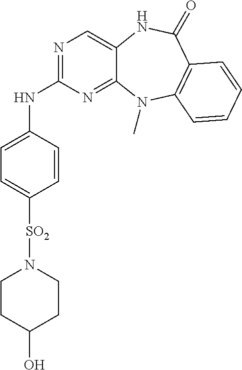 [SMILES: CN1c2ccccc2C(=O)Nc2cnc(Nc3ccc(S(=O)(=O)N4CCC(O)CC4)cc3)nc21] | IA-3 |
| 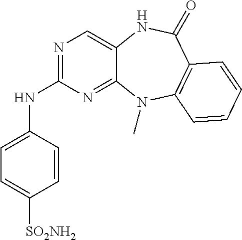 [SMILES: CN1c2ccccc2C(=O)Nc2cnc(Nc3ccc(S(N)(=O)=O)cc3)nc21] | IA-4 |
| (TFA Salt) | |
| 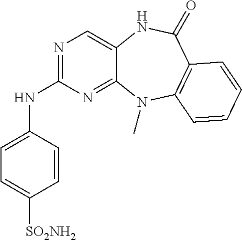 [SMILES: CN1c2ccccc2C(=O)Nc2cnc(Nc3ccc(S(N)(=O)=O)cc3)nc21] | IA-5 |
| (HCl Salt) | |
| 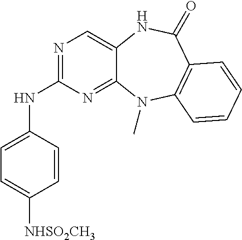 | IA-6 |
| (HCl Salt) | |
| 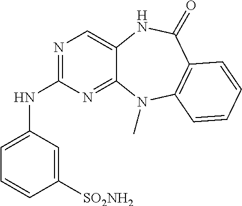 [SMILES: CN1c2ccccc2C(=O)Nc2cnc(Nc3cccc(S(N)(=O)=O)c3)nc21] | IA-7 |
| (HCl Salt) | |
| 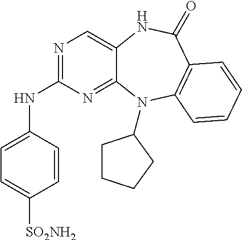 [SMILES: NS(=O)(=O)c1ccc(Nc2ncc3c(n2)N(C2CCCC2)c2ccccc2C(=O)N3)cc1] | IB-1 |
| (TFA Salt) | |
| 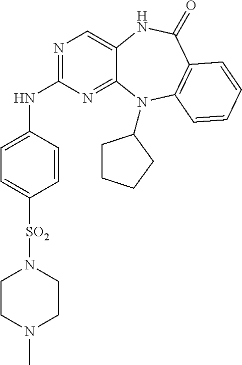 [SMILES: CN1CCN(S(=O)(=O)c2ccc(Nc3ncc4c(n3)N(C3CCCC3)c3ccccc3C(=O)N4)cc2)CC1] | IB-2 |
| (TFA Salt) | |
| 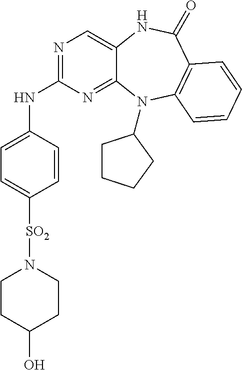 [SMILES: O=C1Nc2cnc(Nc3ccc(S(=O)(=O)N4CCC(O)CC4)cc3)nc2N(C2CCCC2)c2ccccc21] | IB-3 |
| (TFA Salt) | |
| 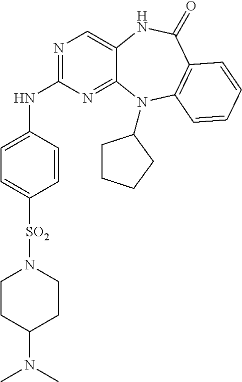 [SMILES: CN(C)C1CCN(S(=O)(=O)c2ccc(Nc3ncc4c(n3)N(C3CCCC3)c3ccccc3C(=O)N4)cc2)CC1] | IB-4 |
| 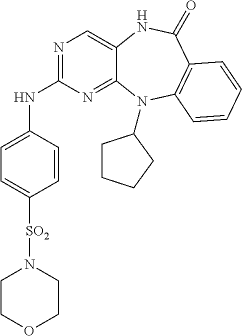 [SMILES: O=C1Nc2cnc(Nc3ccc(S(=O)(=O)N4CCOCC4)cc3)nc2N(C2CCCC2)c2ccccc21] | IB-5 |
| 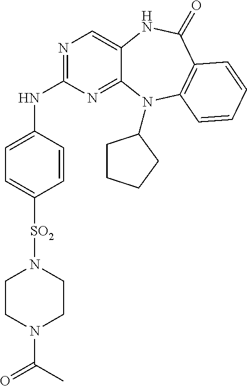 [SMILES: CC(=O)N1CCN(S(=O)(=O)c2ccc(Nc3ncc4c(n3)N(C3CCCC3)c3ccccc3C(=O)N4)cc2)CC1] | IB-6 |
| 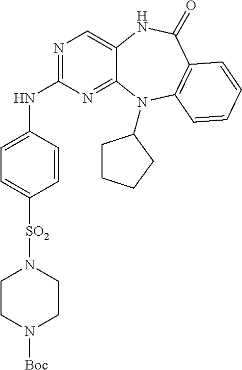 [SMILES: CC(C)(C)OC(=O)N1CCN(S(=O)(=O)c2ccc(Nc3ncc4c(n3)N(C3CCCC3)c3ccccc3C(=O)N4)cc2)CC1] | IB-7 |
| 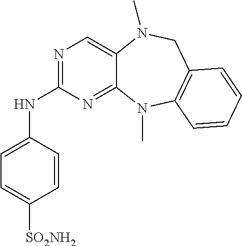 [SMILES: CN1Cc2ccccc2N(C)c2nc(Nc3ccc(S(N)(=O)=O)cc3)ncc21] | IC-1 |
| 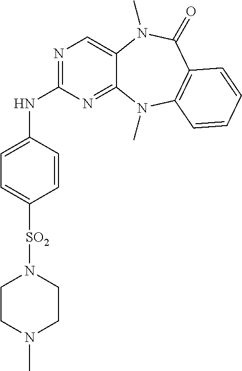 [SMILES: CN1CCN(S(=O)(=O)c2ccc(Nc3ncc4c(n3)N(C)c3ccccc3C(=O)N4C)cc2)CC1] | IC-2 |
| (TFA Salt) | |
| 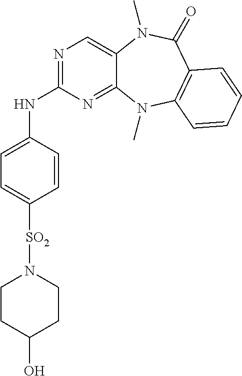 [SMILES: CN1C(=O)c2ccccc2N(C)c2nc(Nc3ccc(S(=O)(=O)N4CCC(O)CC4)cc3)ncc21] | IC-3 |
| (TFA Salt) | |
| 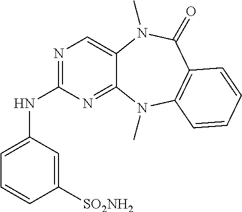 [SMILES: CN1C(=O)c2ccccc2N(C)c2nc(Nc3cccc(S(N)(=O)=O)c3)ncc21] | IC-4 |
| (TFA Salt) | |
| 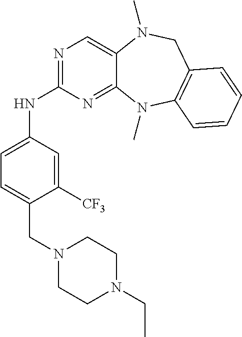 [SMILES: CCN1CCN(Cc2ccc(Nc3ncc4c(n3)N(C)c3ccccc3CN4C)cc2C(F)(F)F)CC1] | IC-5 |
| (TFA Salt) | |
| 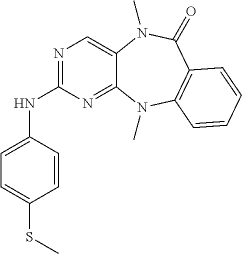 [SMILES: CSc1ccc(Nc2ncc3c(n2)N(C)c2ccccc2C(=O)N3C)cc1] | IC-6 |
| 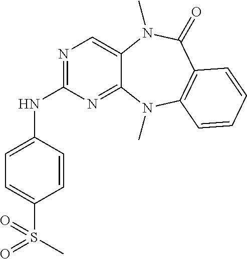 [SMILES: CN1C(=O)c2ccccc2N(C)c2nc(Nc3ccc(S(C)(=O)=O)cc3)ncc21] | IC-7 |
| 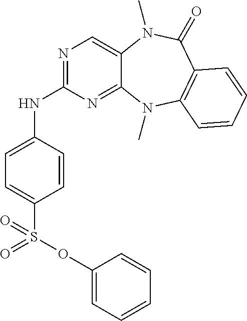 [SMILES: CN1C(=O)c2ccccc2N(C)c2nc(Nc3ccc(S(=O)(=O)Oc4ccccc4)cc3)ncc21] | IC-8 |
| 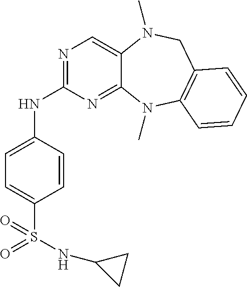 [SMILES: CN1Cc2ccccc2N(C)c2nc(Nc3ccc(S(=O)(=O)NC4CC4)cc3)ncc21] | IC-9 |
| 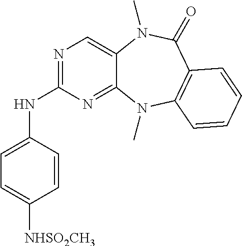 | IC-10 |
| (HCl Salt) | |
| 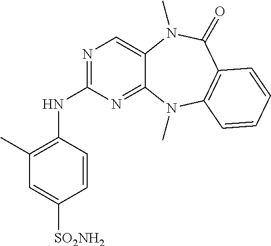 [SMILES: Cc1cc(S(N)(=O)=O)ccc1Nc1ncc2c(n1)N(C)c1ccccc1C(=O)N2C] | IC-11 |
| (TFA Salt) | |
| 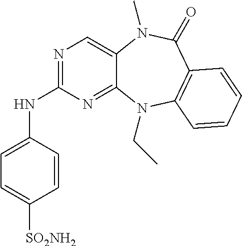 [SMILES: CCN1c2ccccc2C(=O)N(C)c2cnc(Nc3ccc(S(N)(=O)=O)cc3)nc21] | ID-1 |
| (HCl Salt) | |
| 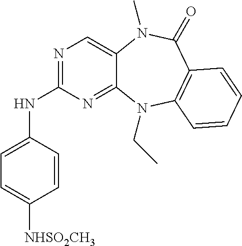 | ID-2 |
| (HCl Salt) | |
| 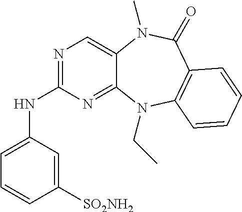 [SMILES: CCN1c2ccccc2C(=O)N(C)c2cnc(Nc3cccc(S(N)(=O)=O)c3)nc21] | ID-3 |
| (HCl Salt) | |
| 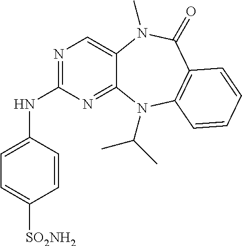 [SMILES: CC(C)N1c2ccccc2C(=O)N(C)c2cnc(Nc3ccc(S(N)(=O)=O)cc3)nc21] | IE-1 |
| (HCl Salt) | |
| 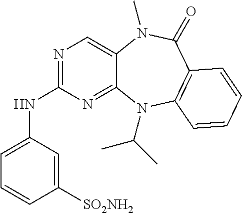 [SMILES: CC(C)N1c2ccccc2C(=O)N(C)c2cnc(Nc3cccc(S(N)(=O)=O)c3)nc21] | IE-2 |
| (TFA Salt) | |
| 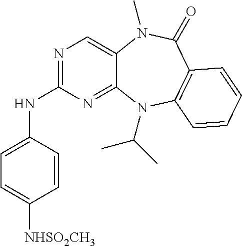 | IE-3 |
| (TFA Salt) | |
| 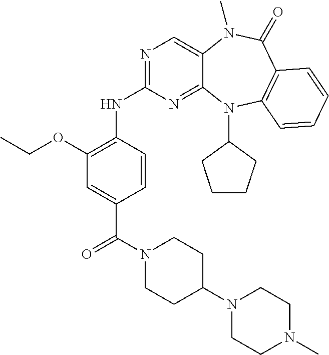 [SMILES: CCOc1cc(C(=O)N2CCC(N3CCN(C)CC3)CC2)ccc1Nc1ncc2c(n1)N(C1CCCC1)c1ccccc1C(=O)N2C] | IF-1 |
| 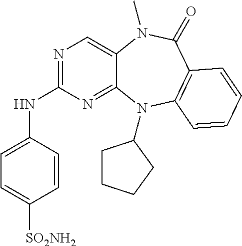 [SMILES: CN1C(=O)c2ccccc2N(C2CCCC2)c2nc(Nc3ccc(S(N)(=O)=O)cc3)ncc21] | IF-2 |
| 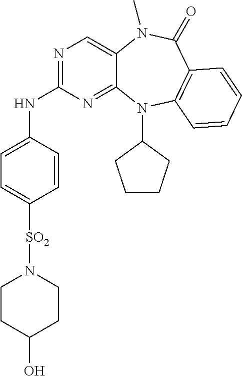 [SMILES: CN1C(=O)c2ccccc2N(C2CCCC2)c2nc(Nc3ccc(S(=O)(=O)N4CCC(O)CC4)cc3)ncc21] | IF-3 |
| 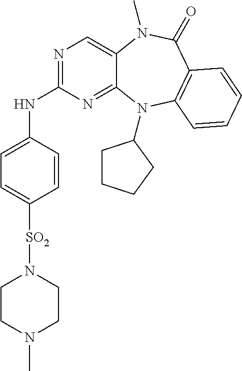 [SMILES: CN1CCN(S(=O)(=O)c2ccc(Nc3ncc4c(n3)N(C3CCCC3)c3ccccc3C(=O)N4C)cc2)CC1] | IF-4 |
| (TFA Salt) | |
| 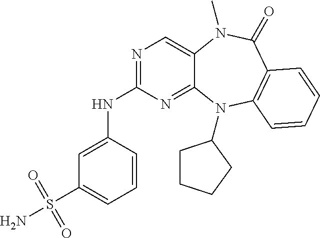 [SMILES: CN1C(=O)c2ccccc2N(C2CCCC2)c2nc(Nc3cccc(S(N)(=O)=O)c3)ncc21] | IF-5 |
| (TFA Salt) | |
| 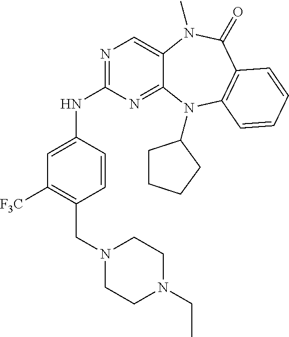 [SMILES: CCN1CCN(Cc2ccc(Nc3ncc4c(n3)N(C3CCCC3)c3ccccc3C(=O)N4C)cc2C(F)(F)F)CC1] | IF-6 |
| 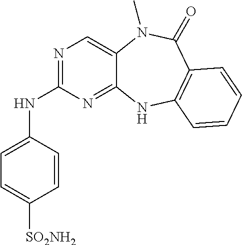 [SMILES: CN1C(=O)c2ccccc2Nc2nc(Nc3ccc(S(N)(=O)=O)cc3)ncc21] | IG-1 |
| (HCl Salt) | |
| 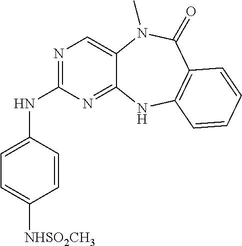 | IG-2 |
| (HCl Salt) | |
| 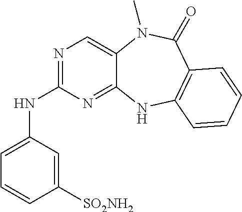 [SMILES: CN1C(=O)c2ccccc2Nc2nc(Nc3cccc(S(N)(=O)=O)c3)ncc21] | IG-3 |
| 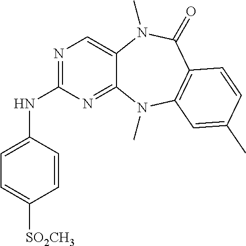 [SMILES: Cc1ccc2c(c1)N(C)c1nc(Nc3ccc(S(C)(=O)=O)cc3)ncc1N(C)C2=O] | IH-1 |
| 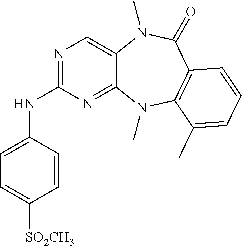 [SMILES: Cc1cccc2c1N(C)c1nc(Nc3ccc(S(C)(=O)=O)cc3)ncc1N(C)C2=O] | IH-2 |
| (TFA Salt) | |
| 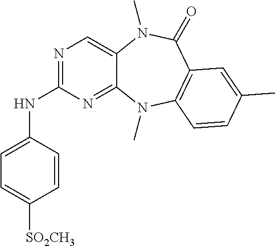 [SMILES: Cc1ccc2c(c1)C(=O)N(C)c1cnc(Nc3ccc(S(C)(=O)=O)cc3)nc1N2C] | IH-3 |
| (TFA Salt) | |
| 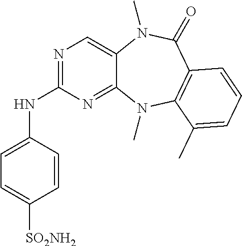 [SMILES: Cc1cccc2c1N(C)c1nc(Nc3ccc(S(N)(=O)=O)cc3)ncc1N(C)C2=O] | IH-4 |
| (TFA Salt) | |
| 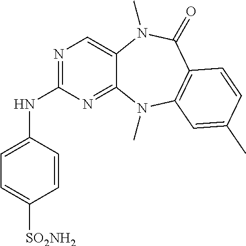 [SMILES: Cc1ccc2c(c1)N(C)c1nc(Nc3ccc(S(N)(=O)=O)cc3)ncc1N(C)C2=O] | IH-5 |
| (TFA Salt) | |
| 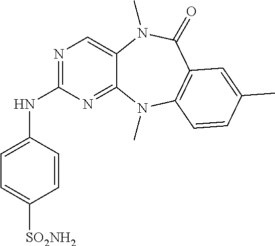 [SMILES: Cc1ccc2c(c1)C(=O)N(C)c1cnc(Nc3ccc(S(N)(=O)=O)cc3)nc1N2C] | IH-6 |
| (HCl Salt) | |
| 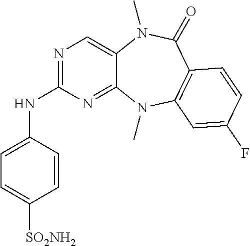 [SMILES: CN1C(=O)c2ccc(F)cc2N(C)c2nc(Nc3ccc(S(N)(=O)=O)cc3)ncc21] | IH-7 |
| (HCl Salt) | |
| 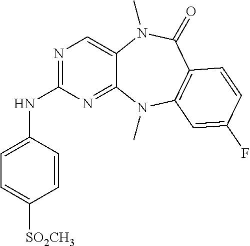 [SMILES: CN1C(=O)c2ccc(F)cc2N(C)c2nc(Nc3ccc(S(C)(=O)=O)cc3)ncc21] | IH-8 |
| 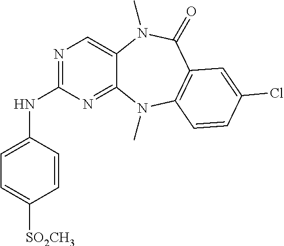 [SMILES: CN1C(=O)c2cc(Cl)ccc2N(C)c2nc(Nc3ccc(S(C)(=O)=O)cc3)ncc21] | IH-9 |
| 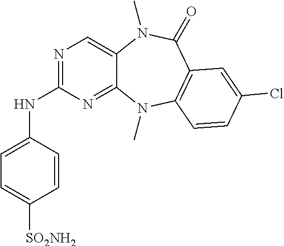 [SMILES: CN1C(=O)c2cc(Cl)ccc2N(C)c2nc(Nc3ccc(S(N)(=O)=O)cc3)ncc21] | IH-10 |
| (HCl Salt) | |
| 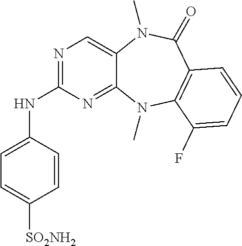 [SMILES: CN1C(=O)c2cccc(F)c2N(C)c2nc(Nc3ccc(S(N)(=O)=O)cc3)ncc21] | IH-11 |
| (HCl Salt) | |
| 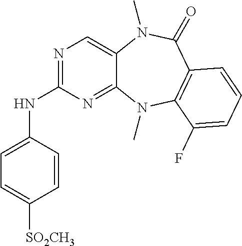 [SMILES: CN1C(=O)c2cccc(F)c2N(C)c2nc(Nc3ccc(S(C)(=O)=O)cc3)ncc21] | IH-12 |
| (HCl Salt) | |
| 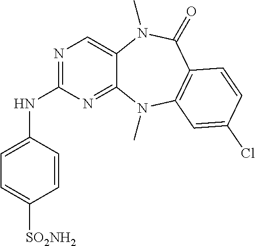 [SMILES: CN1C(=O)c2ccc(Cl)cc2N(C)c2nc(Nc3ccc(S(N)(=O)=O)cc3)ncc21] | IH-13 |
| (HCl Salt) | |
| 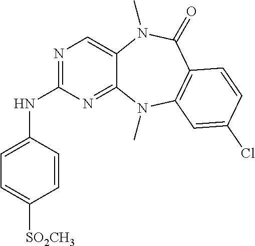 [SMILES: CN1C(=O)c2ccc(Cl)cc2N(C)c2nc(Nc3ccc(S(C)(=O)=O)cc3)ncc21] | IH-14 |
| (HCl Salt) | |
| 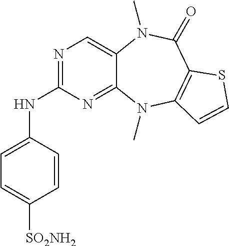 [SMILES: CN1C(=O)c2sccc2N(C)c2nc(Nc3ccc(S(N)(=O)=O)cc3)ncc21] | II-1 |
| (TFA Salt) | |
| 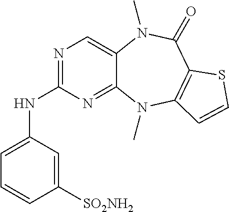 [SMILES: CN1C(=O)c2sccc2N(C)c2nc(Nc3cccc(S(N)(=O)=O)c3)ncc21] | II-2 |
| 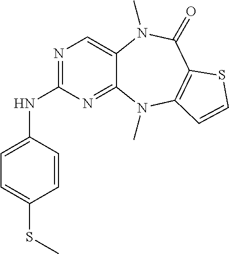 [SMILES: CSc1ccc(Nc2ncc3c(n2)N(C)c2ccsc2C(=O)N3C)cc1] | II-3 |
| 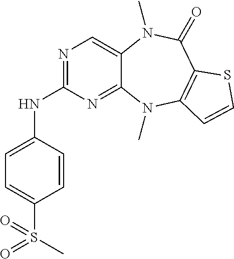 [SMILES: CN1C(=O)c2sccc2N(C)c2nc(Nc3ccc(S(C)(=O)=O)cc3)ncc21] | II-4 |
| 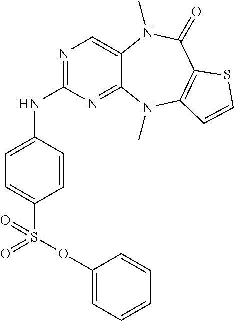 [SMILES: CN1C(=O)c2sccc2N(C)c2nc(Nc3ccc(S(=O)(=O)Oc4ccccc4)cc3)ncc21] | II-5 |
| 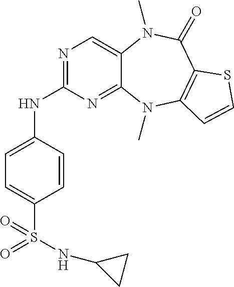 [SMILES: CN1C(=O)c2sccc2N(C)c2nc(Nc3ccc(S(=O)(=O)NC4CC4)cc3)ncc21] | II-6 |
| 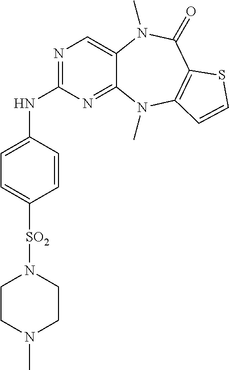 [SMILES: CN1CCN(S(=O)(=O)c2ccc(Nc3ncc4c(n3)N(C)c3ccsc3C(=O)N4C)cc2)CC1] | II-7 |
| 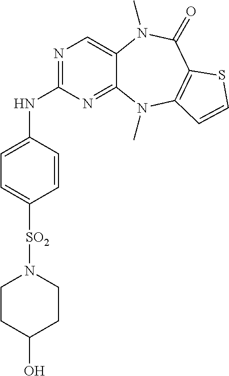 [SMILES: CN1C(=O)c2sccc2N(C)c2nc(Nc3ccc(S(=O)(=O)N4CCC(O)CC4)cc3)ncc21] | II-8 |
| (TFA Salt) | |
| 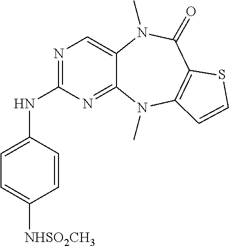 | II-9 |
| 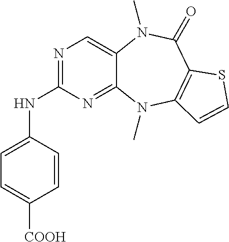 [SMILES: CN1C(=O)c2sccc2N(C)c2nc(Nc3ccc(C(=O)O)cc3)ncc21] | II-10 |
| (HCl Salt) | |
| 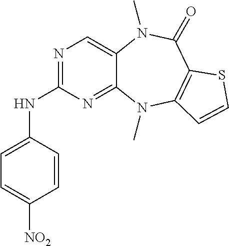 [SMILES: CN1C(=O)c2sccc2N(C)c2nc(Nc3ccc([N+](=O)[O-])cc3)ncc21] | II-11 |
| (HCl Salt) | |
| 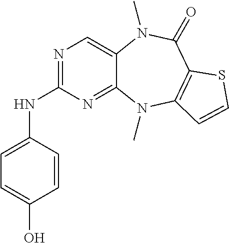 [SMILES: CN1C(=O)c2sccc2N(C)c2nc(Nc3ccc(O)cc3)ncc21] | II-12 |
| (HCl Salt) | |
| 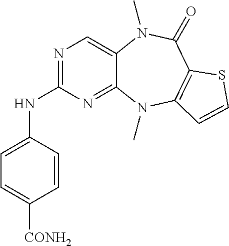 [SMILES: CN1C(=O)c2sccc2N(C)c2nc(Nc3ccc(C(N)=O)cc3)ncc21] | II-13 |
| (HCl Salt) | |
| 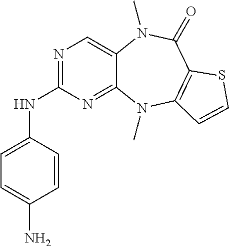 [SMILES: CN1C(=O)c2sccc2N(C)c2nc(Nc3ccc(N)cc3)ncc21] | II-14 |
| (TFA Salt) | |
| 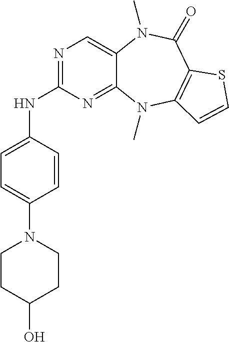 [SMILES: CN1C(=O)c2sccc2N(C)c2nc(Nc3ccc(N4CCC(O)CC4)cc3)ncc21] | II-15 |
| 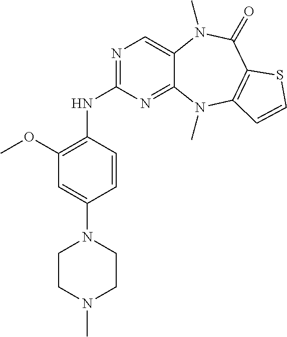 [SMILES: COc1cc(N2CCN(C)CC2)ccc1Nc1ncc2c(n1)N(C)c1ccsc1C(=O)N2C] | II-16 |
| 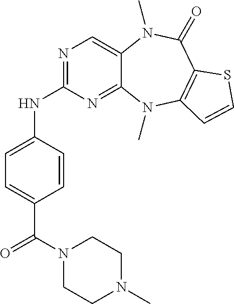 [SMILES: CN1CCN(C(=O)c2ccc(Nc3ncc4c(n3)N(C)c3ccsc3C(=O)N4C)cc2)CC1] | II-17 |
| (TFA Salt) | |
| 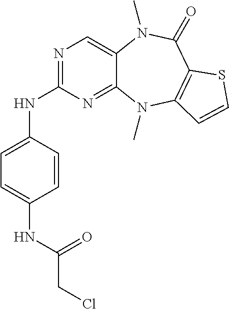 [SMILES: CN1C(=O)c2sccc2N(C)c2nc(Nc3ccc(NC(=O)CCl)cc3)ncc21] | II-18 |
| (TFA Salt) | |
| 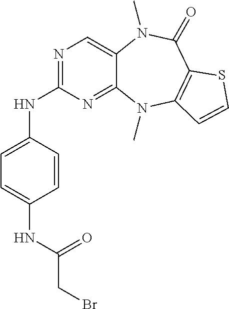 [SMILES: CN1C(=O)c2sccc2N(C)c2nc(Nc3ccc(NC(=O)CBr)cc3)ncc21] | II-19 |
| (TFA Salt) | |
| 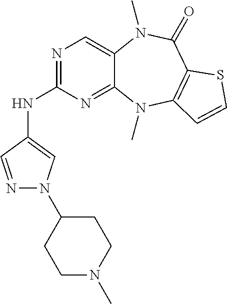 [SMILES: CN1CCC(n2cc(Nc3ncc4c(n3)N(C)c3ccsc3C(=O)N4C)cn2)CC1] | II-20 |
| 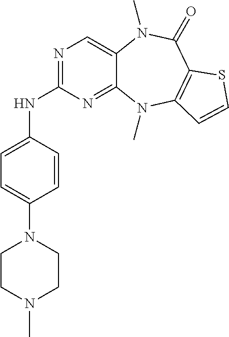 [SMILES: CN1CCN(c2ccc(Nc3ncc4c(n3)N(C)c3ccsc3C(=O)N4C)cc2)CC1] | II-21 |
| 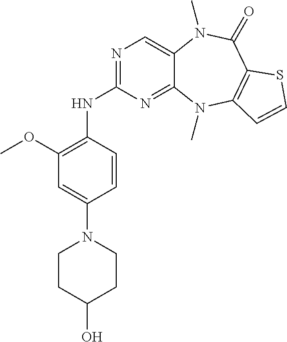 [SMILES: COc1cc(N2CCC(O)CC2)ccc1Nc1ncc2c(n1)N(C)c1ccsc1C(=O)N2C] | II-22 |
| 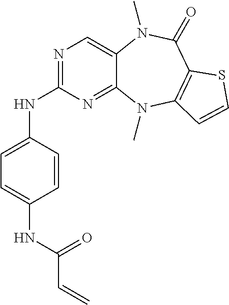 [SMILES: C=CC(=O)Nc1ccc(Nc2ncc3c(n2)N(C)c2ccsc2C(=O)N3C)cc1] | II-23 |
| (TFA Salt) | |
| 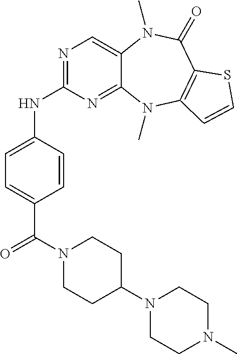 [SMILES: CN1CCN(C2CCN(C(=O)c3ccc(Nc4ncc5c(n4)N(C)c4ccsc4C(=O)N5C)cc3)CC2)CC1] | II-24 |
| 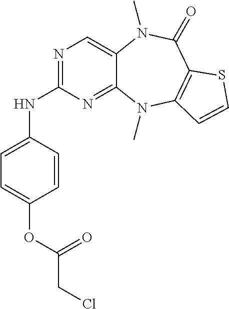 [SMILES: CN1C(=O)c2sccc2N(C)c2nc(Nc3ccc(OC(=O)CCl)cc3)ncc21] | II-25 |
| 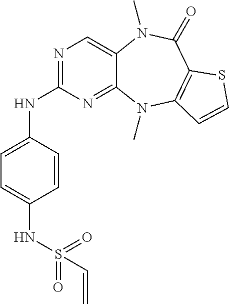 [SMILES: C=CS(=O)(=O)Nc1ccc(Nc2ncc3c(n2)N(C)c2ccsc2C(=O)N3C)cc1] | II-26 |
| (TFA Salt) | |
| 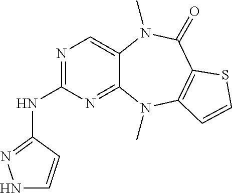 [SMILES: CN1C(=O)c2sccc2N(C)c2nc(Nc3cc[nH]n3)ncc21] | II-27 |
| 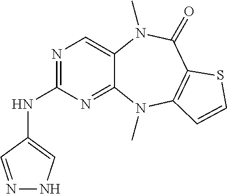 [SMILES: CN1C(=O)c2sccc2N(C)c2nc(Nc3cn[nH]c3)ncc21] | II-28 |
| 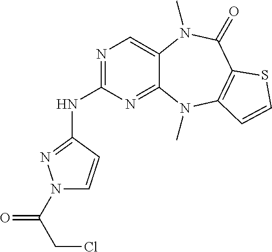 [SMILES: CN1C(=O)c2sccc2N(C)c2nc(Nc3ccn(C(=O)CCl)n3)ncc21] | I-29 |
| 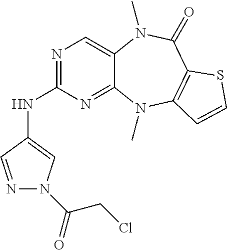 [SMILES: CN1C(=O)c2sccc2N(C)c2nc(Nc3cnn(C(=O)CCl)c3)ncc21] | II-30 |
| 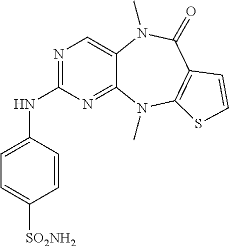 [SMILES: CN1C(=O)c2ccsc2N(C)c2nc(Nc3ccc(S(N)(=O)=O)cc3)ncc21] | III-1 |
| (TFA Salt) | |
| 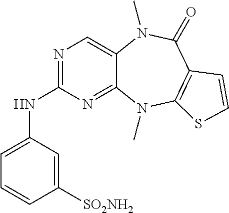 [SMILES: CN1C(=O)c2ccsc2N(C)c2nc(Nc3cccc(S(N)(=O)=O)c3)ncc21] | III-2 |
| 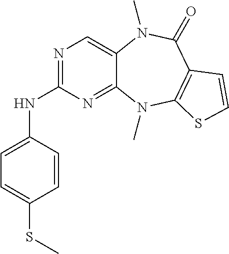 [SMILES: CSc1ccc(Nc2ncc3c(n2)N(C)c2sccc2C(=O)N3C)cc1] | III-3 |
| 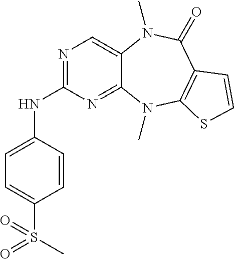 [SMILES: CN1C(=O)c2ccsc2N(C)c2nc(Nc3ccc(S(C)(=O)=O)cc3)ncc21] | III-4 |
| 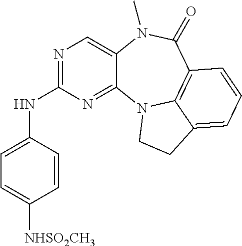 | IV-1 |
| (TFA Salt) | |
| 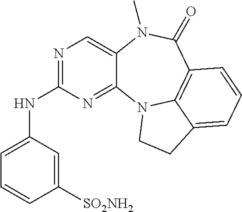 [SMILES: CN1C(=O)c2cccc3c2N(CC3)c2nc(Nc3cccc(S(N)(=O)=O)c3)ncc21] | IV-2 |
| (TFA Salt) | |
| 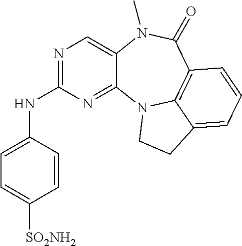 [SMILES: CN1C(=O)c2cccc3c2N(CC3)c2nc(Nc3ccc(S(N)(=O)=O)cc3)ncc21] | IV-3 |
| (TFA Salt) | |

30. The methods of claim 1, wherein the MST1/2 protein kinase inhibitor is selected from:

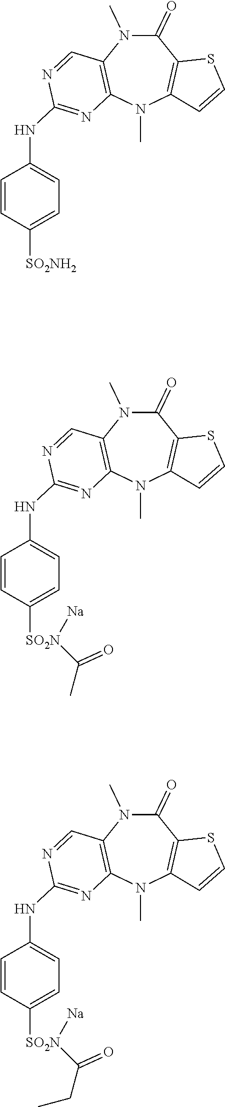

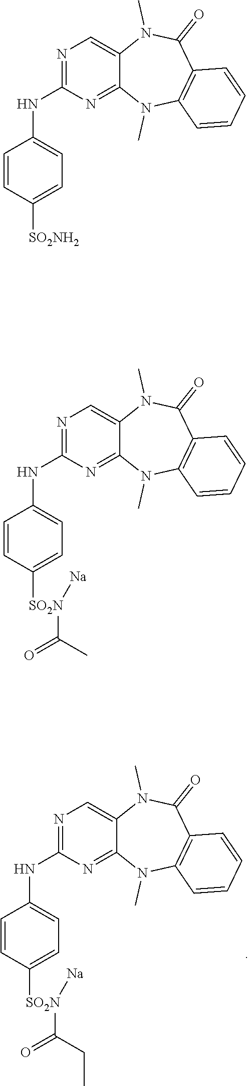

31-34. (canceled)
